SEQUENCE LISTING (55 sequences)

SEQ ID NO: 1 - protein, 8 aa, synthetic construct
  REGION 1..8: note = CDR1 of B1
GRLSSSNS

SEQ ID NO: 2 - protein, 7 aa, synthetic construct
  REGION 1..7: note = CDR2 of B1
ITGGGET

SEQ ID NO: 3 - protein, 9 aa, synthetic construct
  REGION 1..9: note = CDR3 of B1
NFWPPLINY

SEQ ID NO: 4 - protein, 115 aa, synthetic construct
  REGION 1..115: note = B1
DVQLVESGGGLVQPGGSLRLSCVASGRLSSSNSMAWYRQVPGKRRELVAGITGGGETNYADFVGGRFTISRDNAKNGLYLQLNGLKPEDTAAYYCNFWPPLINYWGQGTQVTVSS

SEQ ID NO: 5 - protein, 8 aa, synthetic construct
  REGION 1..8: note = CDR1 of B2
GSISSANS

SEQ ID NO: 6 - protein, 7 aa, synthetic construct
  REGION 1..7: note = CDR2 of B2
LTTGGRS

SEQ ID NO: 7 - protein, 9 aa, synthetic construct
  REGION 1..9: note = CDR3 of B2
NLWPPVQGY

SEQ ID NO: 8 - protein, 115 aa, synthetic construct
  REGION 1..115: note = B2
DVQLVESGGGLVQAGGSLRLSCAVSGSISSANSMGWYRQAPGKQRDVVAGLTTGGRSHYADSVKGRFTISRDNAKNTVYLQMNSLKAEDTAVYYCNLWPPVQGYWGQGTQVTVSS

SEQ ID NO: 9 - protein, 8 aa, synthetic construct
  REGION 1..8: note = CDR1 of B3
GRLFSTNA

SEQ ID NO: 10 - protein, 7 aa, synthetic construct
  REGION 1..7: note = CDR2 of B3
ITGGDRS

SEQ ID NO: 11 - protein, 10 aa, synthetic construct
  REGION 1..10: note = CDR3 of B3
NFYPPIVGDY

SEQ ID NO: 12 - protein, 116 aa, synthetic construct
  REGION 1..116: note = B3
DVQLVESGGGLVQAGGSLRLSCAVSGRLFSTNAMGWYRQAPGKQRELVAGITGGDRSNYADSVKGRFTISRDNGKNTLYLQMNSLKPEDTAVYYCNFYPPIVGDYWGQGTQVTVSS

SEQ ID NO: 13 - protein, 8 aa, synthetic construct
  REGION 1..8: note = CDR1 of B4
GFTFSSYY

SEQ ID NO: 14 - protein, 8 aa, synthetic construct
  REGION 1..8: note = CDR2 of B4
IYADGDMT

SEQ ID NO: 15 - protein, 9 aa, synthetic construct
  REGION 1..9: note = CDR3 of B4
AKDPLPPYH

SEQ ID NO: 16 - protein, 116 aa, synthetic construct
  REGION 1..116: note = B4
DVQLVESGGGLVQVGGSLRLSCVASGFTFSSYYMSWVRQAPGKGLEWVASIYADGDMTYYADSVRGRFTISRDNAKNTLYLQMNSLKSEDTAVYYCAKDPLPPYHVNQGTQVTVSS

SEQ ID NO: 17 - protein, 134 aa, synthetic construct
  REGION 1..134: note = B1 with HA-His6-tag
QVQLQESGGGLVQPGGSLRLSCVASGRLSSSNSMAWYRQVPGKRRELVAGITGGGETNYADFVGGRFTISRDNAKNGLYLQLNGLKPEDTAAYYCNFWPPLINYWGQGTQVTVSSAAAYPYDVPDYGSHHHHHH

SEQ ID NO: 18 - protein, 134 aa, synthetic construct
  REGION 1..134: note = B2 with HA-His6-tag
QVQLQESGGGLVQAGGSLRLSCAVSGSISSANSMGWYRQAPGKQRDVVAGLTTGGRSHYADSVKGRFTISRDNAKNTVYLQMNSLKAEDTAVYYCNLWPPVQGYWGQGTQVTVSSAAAYPYDVPDYGSHHHHHH

SEQ ID NO: 19 - protein, 135 aa, synthetic construct
  REGION 1..135: note = B3 with HA-His6-tag
QVQLQESGGGLVQAGGSLRLSCAVSGRLFSTNAMGWYRQAPGKQRELVAGITGGDRSNYADSVKGRFTISRDNGKNTLYLQMNSLKPEDTAVYYCNFYPPIVGDYWGQGTQVTVSSAAAYPYDVPDYGSHHHHHH

SEQ ID NO: 20 - protein, 135 aa, synthetic construct
  REGION 1..135: note = B4 with HA-His6-tag
QVQLQESGGGLVQVGGSLRLSCVASGFTFSSYYMSWVRQAPGKGLEWVASIYADGDMTYYADSVRGRFTISRDNAKNTLYLQMNSLKSEDTAVYYCAKDPLPPYHVNQGTQVTVSSAAAYPYDVPDYGSHHHHHH

SEQ ID NO: 21 - protein, 121 aa, synthetic construct
  REGION 1..121: note = B1 with His6-tag
DVQLVESGGGLVQPGGSLRLSCVASGRLSSSNSMAWYRQVPGKRRELVAGITGGGETNYADFVGGRFTISRDNAKNGLYLQLNGLKPEDTAAYYCNFWPPLINYWGQGTQVTVSSHHHHHH

SEQ ID NO: 22 - protein, 121 aa, synthetic construct
  REGION 1..121: note = B2 with His6-tag
DVQLVESGGGLVQAGGSLRLSCAVSGSISSANSMGWYRQAPGKQRDVVAGLTTGGRSHYADSVKGRFTISRDNAKNTVYLQMNSLKAEDTAVYYCNLWPPVQGYWGQGTQVTVSSHHHHHH

SEQ ID NO: 23 - protein, 122 aa, synthetic construct
  REGION 1..122: note = B3 with His6-tag
DVQLVESGGGLVQAGGSLRLSCAVSGRLFSTNAMGWYRQAPGKQRELVAGITGGDRSNYADSVKGRFTISRDNGKNTLYLQMNSLKPEDTAVYYCNFYPPIVGDYWGQGTQVTVSSHHHHHH

SEQ ID NO: 24 - protein, 122 aa, synthetic construct
  REGION 1..122: note = B4 with His6-tag
DVQLVESGGGLVQVGGSLRLSCVASGFTFSSYYMSWVRQAPGKGLEWVASIYADGDMTYYADSVRGRFTISRDNAKNTLYLQMNSLKSEDTAVYYCAKDPLPPYHVNQGTQVTVSSHHHHHH

SEQ ID NO: 25 - DNA, 2283 bp, Homo sapiens
  misc_feature 1..2283: note = nucleic acid encoding human fibroblast activation protein alpha (FAP), transcript variant 1
  source 1..2283: mol_type = genomic DNA
atgaagacttgggtaaaaatcgtatttggagttgccacctctgctgtgcttgccttattggtgatgtgcattgtcttacgcccttcaagagttcataactctgaagaaaatacaatgagagcactcacactgaaggatattttaaatggaacattttcttataaaacattttttccaaactggatttcaggacaagaatatcttcatcaatctgcagataacaatatagtactttataatattgaaacaggacaatcatataccattttgagtaatagaaccatgaaaagtgtgaatgcttcaaattacggcttatcacctgatcggcaatttgtatatctagaaagtgattattcaaagctttggagatactcttacacagcaacatattacatctatgaccttagcaatggagaatttgtaagaggaaatgagcttcctcgtccaattcagtatttatgctggtcgcctgttgggagtaaattagcatatgtctatcaaaacaatatctatttgaaacaaagaccaggagatccaccttttcaaataacatttaatggaagagaaaataaaatatttaatggaatcccagactgggtttatgaagaggaaatgcttgctacaaaatatgctctctggtggtctcctaatggaaaatttttggcatatgcggaatttaatgatacggatataccagttattgcctattcctattatggcgatgaacaatatcctagaacaataaatattccatacccaaaggctggagctaagaatcccgttgttcggatatttattatcgataccacttaccctgcgtatgtaggtccccaggaagtgcctgttccagcaatgatagcctcaagtgattattatttcagttggctcacgtgggttactgatgaacgagtatgtttgcagtggctaaaaagagtccagaatgtttcggtcctgtctatatgtgacttcagggaagactggcagacatgggattgtccaaagacccaggagcatatagaagaaagcagaactggatgggctggtggattctttgtttcaacaccagttttcagctatgatgccatttcgtactacaaaatatttagtgacaaggatggctacaaacatattcactatatcaaagacactgtggaaaatgctattcaaattacaagtggcaagtgggaggccataaatatattcagagtaacacaggattcactgttttattctagcaatgaatttgaagaataccctggaagaagaaacatctacagaattagcattggaagctatcctccaagcaagaagtgtgttacttgccatctaaggaaagaaaggtgccaatattacacagcaagtttcagcgactacgccaagtactatgcacttgtctgctacggcccaggcatccccatttccacccttcatgatggacgcactgatcaagaaattaaaatcctggaagaaaacaaggaattggaaaatgctttgaaaaatatccagctgcctaaagaggaaattaagaaacttgaagtagatgaaattactttatggtacaagatgattcttcctcctcaatttgacagatcaaagaagtatcccttgctaattcaagtgtatggtggtccctgcagtcagagtgtaaggtctgtatttgctgttaattggatatcttatcttgcaagtaaggaagggatggtcattgccttggtggatggtcgaggaacagctttccaaggtgacaaactcctctatgcagtgtatcgaaagctgggtgtttatgaagttgaagaccagattacagctgtcagaaaattcatagaaatgggtttcattgatgaaaaaagaatagccatatggggctggtcctatggaggatacgtttcatcactggcccttgcatctggaactggtcttttcaaatgtggtatagcagtggctccagtctccagctgggaatattacgcgtctgtctacacagagagattcatgggtctcccaacaaaggatgataatcttgagcactataagaattcaactgtgatggcaagagcagaatatttcagaaatgtagactatcttctcatccacggaacagcagatgataatgtgcactttcaaaactcagcacagattgctaaagctctggttaatgcacaagtggatttccaggcaatgtggtactctgaccagaaccacggcttatccggcctgtccacgaaccacttatacacccacatgacccacttcctaaagcagtgtttctctttgtcagactaa

SEQ ID NO: 26 - protein, 760 aa, Homo sapiens
  REGION 1..760: note = human fibroblast activation protein alpha (FAP), transcript variant 1
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 27 - DNA, 17086 bp, synthetic construct
  misc_feature 1..17086: note = targeting vector
  source 1..17086: mol_type = other DNA
cgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcccgggctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctaggatctgcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagagatcgcctgacacgatttcctgcacaggcttgagccatatactcatacatcgcatcttggccacgttttccacgggtttcaaaattaatctcaagttctacgcttaacgctttcgcctgttcccagttattaatatattcaacgctagaactcccctcagcgaagggaaggctgagcactacacgcgaagcaccatcaccgaaccttttgataaactcttccgttccgacttgctccatcaacggttcagtgagacttaaacctaactctttcttaatagtttcggcattatccacttttagtgcgagaaccttcgtcagtcctggatacgtcactttgaccacgcctccagcttttccagagagcgggttttcattatctacagagtatcccgcagcgtcgtatttattgtcggtactataaaaccctttccaatcatcgtcataatttccttgtgtaccagattttggcttttgtatacctttttgaatggaatctacataaccaggtttagtcccgtggtacgaagaaaagttttccatcacaaaagatttagaagaatcaacaacatcatcaggatccatggcacgcgcttctacaaggcgctggccgaagaggtgcgggagtttcacgccaccaagatctgcggcacgctgttgacgctgttaagcgggtcgctgcagggtcgctcggtgttcgaggccacacgcgtcaccttaatatgcgaagtggacctgggaccgcgccgccccgactgcatctgcgtgttcgaattcgccaatgacaagacgctgggcggggtttgctcgacattgggtggaaacattccaggcctgggtggagaggctttttgcttcctcttgcaaaaccacactgctcgacattgggtggaaacattccaggcctgggtggagaggctttttgcttcctcttgaaaaccacactgctcgatttgttagcagcctcgaatcaacccgggcgatcctaggcgatgagatctagctgtcgcgaagagtggcgcgccacccagtcacatcctccttaaaacagttcaaacagctagaactatcccagatgttctagttagattcaaccagctttctaaagaaaatacaaacataaataaaatgtttattcaaccttaaaatgtccccgtatactagtactttcaacttaatgttgcttcttaagatgtcccaatttctccataaaacctgtcagacgagtctttggagtcctaagtggtacccattaccagtcatgtgacaatgattaattgctaaagctagcagtatgcattttaattataccttaatcacttattgcatttagtttctgtctagtaaaggaggtcttgggaatcactcactacaaaggacttgctgatttttttgggggtggggggtgggggggtgggcgttggcgagctgggacagaagttctgagaaagtactatgtggatgttccaaagaaagcaacaaatttgagaagaccctctagagggcagttcacatgggacccggctgatggctttctctcggattgacgcctcacaatcaaggcacatggagatgtcagtaagtgcactgtacacctgtgatgatgcctggtactgcttcaatttgtaaatcgatagctcattaaccgcatggggagtgggattcatccgggagctgcagtgggctttgaatactcttctctgccaatgcatactagtgactcactctgctcaccagaactttcctaaggctagaattcagggacagctgtctggagaatggcagggaggaagttcacaagccactgtgctggtttgtggtggagaaagtttcctgtggcagccagaccagtcaactaaagcaaacagcaacacagaacagtgatgttttctcgttatttttcatgcacagtttgtgaaagatcagcatgaaacttagcatagtcaggcaaaagatattacaaacttcttgagaaagccagcaatggaaaaactggagacaccatattctagcaacagtatccatgtggaaaccaggaatctgccctaggaaattgctctggaccacagagaaggtttaaaaaaaaattgtgtctgtgtgtgtgtgtgtgtgtgtgtgtgtgttgtgaattcagtcattttactgatctgccaagatgaccgatcactttgaaatatgtggacttagccaaaatgaagtacacatgacttctcttttcacttgcctttttaaaatttattttaaggtctttataattactaaatgttctcctttctcagaagatattctgagagcaaagcaaaaatatcactcttgtaaagccatttccattcttccaaaggtctgctggcaaattattcccgctgatctttccatctttctagcctgtgcaaacacacctaacccatactacatttcaacagatggcgcttcatttctttaaaggaaagcagccgtgggtttagacagttgaatttttaaacttctgtatttactgaaagtgcatatggtgctatacggacaaagaaattgtgctgaaagaaaaacatttgtgcctgcagcacctcataggctcccgggagaagagtgtgcagtcacaccgtgtacctctcacaaagccggatttggctccactgttcttcccttccccaaacatttgatatgtacaatgcaccctcaaaaagtttctccatttgaaatccttcctctcagctgcaaacaagaggagtgtagaaaagattgttactaaagtgactcgagtgatttttgaggagaatatgaaatataaacctgaacaacaacaaaaaggtgaaataaaacggcctctgtatgacaacttaagcagatgttggtgtaaatttacaaggatgagagagctctagagcctccctccagccactcatagttcacttaaaggccaagaacgcccccaaaatctgtttctaattttacagaaatctttcgaaatttggcacggtagtcaaaagtccgtgggaaagggaggaggggagcgggaagcaactcatgtcctgacttcagcttccaagtggaagacagacttgcttcttttcaacagttttcacagatgcggtgacccacgctgtgcagtgagaatcagctaacttgcaaaaacatctggaaaaatgaagacttgggtaaaaatcgtatttggagttgccacctctgctgtgcttgccttattggtgatgtgcattgtcttacgcccttcaagagttcataactctgaagaaaatacaatgagagcactcacactgaaggatattttaaatggaacattttcttataaaacattttttccaaactggatttcaggacaagaatatcttcatcaatctgcagataacaatatagtactttataatattgaaacaggacaatcatataccattttgagtaatagaaccatgaaaagtgtgaatgcttcaaattacggcttatcacctgatcggcaatttgtatatctagaaagtgattattcaaagctttggagatactcttacacagcaacatattacatctatgaccttagcaatggagaatttgtaagaggaaatgagcttcctcgtccaattcagtatttatgctggtcgcctgttgggagtaaattagcatatgtctatcaaaacaatatctatttgaaacaaagaccaggagatccaccttttcaaataacatttaatggaagagaaaataaaatatttaatggaatcccagactgggtttatgaagaggaaatgcttgctacaaaatatgctctctggtggtctcctaatggaaaatttttggcatatgcggaatttaatgatacggatataccagttattgcctattcctattatggcgatgaacaatatcctagaacaataaatattccatacccaaaggctggagctaagaatcccgttgttcggatatttattatcgataccacttaccctgcgtatgtaggtccccaggaagtgcctgttccagcaatgatagcctcaagtgattattatttcagttggctcacgtgggttactgatgaacgagtatgtttgcagtggctaaaaagagtccagaatgtttcggtcctgtctatatgtgacttcagggaagactggcagacatgggattgtccaaagacccaggagcatatagaagaaagcagaactggatgggctggtggattctttgtttcaacaccagttttcagctatgatgccatttcgtactacaaaatatttagtgacaaggatggctacaaacatattcactatatcaaagacactgtggaaaatgctattcaaattacaagtggcaagtgggaggccataaatatattcagagtaacacaggattcactgttttattctagcaatgaatttgaagaataccctggaagaagaaacatctacagaattagcattggaagctatcctccaagcaagaagtgtgttacttgccatctaaggaaagaaaggtgccaatattacacagcaagtttcagcgactacgccaagtactatgcacttgtctgctacggcccaggcatccccatttccacccttcatgatggacgcactgatcaagaaattaaaatcctggaagaaaacaaggaattggaaaatgctttgaaaaatatccagctgcctaaagaggaaattaagaaacttgaagtagatgaaattactttatggtacaagatgattcttcctcctcaatttgacagatcaaagaagtatcccttgctaattcaagtgtatggtggtccctgcagtcagagtgtaaggtctgtatttgctgttaattggatatcttatcttgcaagtaaggaagggatggtcattgccttggtggatggtcgaggaacagctttccaaggtgacaaactcctctatgcagtgtatcgaaagctgggtgtttatgaagttgaagaccagattacagctgtcagaaaattcatagaaatgggtttcattgatgaaaaaagaatagccatatggggctggtcctatggaggatacgtttcatcactggcccttgcatctggaactggtcttttcaaatgtggtatagcagtggctccagtctccagctgggaatattacgcgtctgtctacacagagagattcatgggtctcccaacaaaggatgataatcttgagcactataagaattcaactgtgatggcaagagcagaatatttcagaaatgtagactatcttctcatccacggaacagcagatgataatgtgcactttcaaaactcagcacagattgctaaagctctggttaatgcacaagtggatttccaggcaatgtggtactctgaccagaaccacggcttatccggcctgtccacgaaccacttatacacccacatgacccacttcctaaagcagtgtttctctttgtcagactaatcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatcctcgagggacctaataacttcgtatagcatacattatacgaagttatattaagggttccgcaagctctagtcgagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagagaattgatcccctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcatcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatggccgatcccatggtttagttcctcaccttgtcgtattatactatgccgatatactatgccgatgattaattgtcaacaggctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgccttgggaaaagcgcctcccctacccggtagaatttcgacgacctgcagccaagctagcttcgcgagctcgaccgaacaaacgacccaacacccgtgcgttttattctgtctttttattgccgctcagctttacagtgacaatgacggctggcgactgaatattagtgcttacagacagcactacatattttccgtcgatgttgaaatcctttctcatatgtcaccataaatatcaaataattatagcaatcatttacgcgttaatggctaatcgccatcttccagcaggcgcaccattgcccctgtttcactatccaggttacggatatagttcatgacaatatttacattggtccagccaccagcttgcatgatctccggtattgaaactccagcgcgggccatatctcgcgcggctccgacacgggcactgtgtccagaccaggccaggtatctctgaccagagtcatccttagcgccgtaaatcaatcgatgagttgcttcaaaaatcccttccagggcgcgagttgatagctggctggtggcagatggcgcggcaacaccattttttctgacccggcaaaacaggtagttattcggatcatcagctacaccagagacggaaatccatcgctcgaccagtttagttacccccaggctaagtgccttctctacacctgcggtgctaaccagcgttttcgttctgccaatatggattaacattctcccaccgtcagtacgtgagatatctttaaccctgatcctggcaatttcggctatacgtaacagggtgttataagcaatccccagaaatgccagattacgtatatcctggcagcgatcgctattttccatgagtgaacgaacctggtcgaaatcagtgcgttcgaacgctagagcctgttttgcacgttcaccggcatcaacgttttcttttcggatccgccgcataaccagtgaaacagcattgctgtcacttggtcgtggcagcccggaccgacgatgaagcatgtttagctggcccaaatgttgctggatagtttttactgccagaccgcgcgcctgaagatatagaagataatcgcgaacatcttcaggttctgcgggaaaccatttccggttattcaacttgcaccatgccgcccacgaccggcaaacggacagaagcattttccaggtatgctcagaaaacgcctggcgatccctgaacatgtccatcaggttcttgcgaacctcatcactcgttgcatcgaccggtaatgcaggcaaattttggtgtacggtcagtaaattggacaccttcctcttcttcttgggcatggccgcaggaaagcagagccctgaagctcccatcaccggccaataagagccaagcctgcagtgtgacctcatagagcaatgtgccagccagcctgaccccaagggccctcaggcttgggcacactgtctctaggaccctgagagaaagacatacccatttctgcttagggccctgaggatgagcccaggggtggcttggcactgaagcaaaggacactggggctcagctggcagcaaagtgaccaggatgctgaggctttgacccagaagccagaggccagaggccaggacttctcttggtcccagtccaccctcactcagagctttaccaatgccctctggatagttgtcgggtaacggtggacgccactgattctctggccagcctaggacttcgccattccgctgattctgctcttccagccactggctgaccggttggaagtactccagcagtgccttggcatccagggcatctgagcctaccaggtccttcagtacctcctgccagggcctggagcagccagcctgcaacacctgcctgccaagcagagtgaccactgtgggcacaggggacacagggtggggcccacaacagcaccattgtccacttgtccctcactagtaaaagaactctagggttgcggggggtgggggaggtctctgtgaggctggtaagggatatttgcctggcccatggagctagcttggctggacgtaaactcctcttcagacctaataacttcgtatagcatacattatacgaagttatattaagggttattgaatatgatcggaattgggctgcaggaattcgatagcttggctgcaggtcgacactggcgatgtaagggtaggaagagctttacagtgttatttcagcaacagaaaaacctgagagctattctgtacccctcccaccacccccccaaaatacacgcagaagacaagctttgataggcctctcaagactgtatttttaactagcattgttttctattactagatttgcctggtagcaagaaaaaaaataatgagtaagaatagaaacgttaaaaaaagaacctatggaggctttgaaatcactaaatagtgcttccaaaaaatttaaggaagatgttcagaatagaaatctcaggaatagccatataggaaaagtaaaactcttgttctatatatagtagcaattagtcattcaattaagaggcttgttacagttacgcaaacattatcaaggtaaattggttggggtgtttgtttgtttgtttgtttgttttttatttttacagaaaggcttaggctggcaacaatgtcacttgactgagacctagaagatgttatgcactgattttgaaactagttttaattttaatattatccattggtaataaactatgcccctattattccaaaccttcttatgaacagcaatgtatgcttctaaagcagagatgtaatgtaacagtgacagagactgtactataggatctgggaagaacaaatgctccgagttgatcagaaccagggaacgtctgtggttaaagtgaacggactctgaacaagggttagtggtccagcctgaaccagcaaacacgtcagtcctgaattctctgttgctttttaaaattgaaacatgtacaacgtacaaaagcttgaagagttcaaaaatgtccacatcctttcacagaattgcaatgtgcaggataacctgatgaaggcagtctccttggagctgcacttttaatttttagcttgcatcttcatgtcctaaagcacaagatttcccctgggccatcaaggtgtatcctcctattgtcattttattaagctagactctcacctcaattcttagaggacacactgcctccaaccaggagcttctgagtgcctggccaggtctctgacccctttaatccagtctagtagaaatcagcctctggcttttgcatacacttaccagagcttgagatggatattctgctccctcaaaaattaacagtttttggaagcaaaagtacatgaattttgttgagacagacttttctgtccacaagtgttgccctttggactttgaagaatattcccaatgagaagcatatttatccattgcaaatagcatataaggatagatatttgggtctatacttacatttatagatatagacatttgtgtaattacatataatcccaatctttatcttctttccaagtttttttatgaatagcatactcaaaaatatgttataaactatatataacataaaagtcctgtgtttttttataacatagatttctattttcaatggaaaagataattctctaagtaatttcctgttattgtgaaataatcaaattaacctcaagatgaattattttgcattcatttacttcttctgcagaagtaaacaatgcttaggcctgctgtctggactgtaaagtcactgcctcttcattattacggacacctcagcatgtcacctgagcagcattgtcttcttgtgcccataccttcaccatgtccaggctggggaccaggcccaactttactcacatttcttccatgggaaggaatgaagtaaaaagtatgaaaaagtaaagacttcccatgggaagtcaaggaaaagtagcaatggaacagaaaggagaagaaaaagctaggtaggcccctataccacttcgtcagaaatgtcaccactgtacgtttgtctcctgtgcccttgggttgctagtccaggagctacttgacaaggtaacagaagtcaagaaatatccatctattccattatttataaactaaatgtactgggttccccaaaatacctcctatcacttgctgttgctgaagttacaaagttcagtagtctcaatttccctcagacaggcaaacaaggaataatttgtcattctacacaccctaaaacacatggccacagccatcattatggagaaatgtctctgtcgctcaagtataagtgaaatgaatagagttggggcgtaggggtcttttcattcccaggtggaagtccacaaagaatcagccttcactttagagaagttttggggggggggggggagagagcagacacagacagtaagaatgagtggaagtggagtagtgaatgagaatgcctgtttaactcacttacatattaaaaataaatgtgttgcagctctttctggaaatcctaaggtatttcattttcattttaattttaatctaaactgaagacaaacatgcctttgtgccctgcaggccatgtggtccttggcttctgccccttcacctgactgactccctttattttcctgcctccatttattctctcttgaggatttttttttccttgcacccttacaccagtatgaagatggaaccaggtaaaaagtcagcacaatatcccttggcttccctttgtctaccctgatgccaatcatacagactttcttgcttcaagagatttacaaagatgatgaaaataaaaaccagaactagaaagtttcagaggccttagaattaaaaaaaaaatctctatagccttctttgagtcatgatttacattttatgaatatcaccctttcttcctccatttgtttgtttgtttgtttattggtttttgtggtacattaccctatggagtgatatataagcactcaatacatgttagctataattttacacatttatttattttatatgtgtgcctctgtgtatgtatgtaggcatggttgtaaggatgtcagagggcaatctgtaggggttgattctttccttctaccataggagttcctgagaccaaactcaggtagtcaggcctactggctgtcacctttacttgctaaaccatctcaatttcccaactatgatttttttaatttcaaaatttatcacacaaaagaaattgtgcctacaactctctctcttttcttttacctcctaatatggggatctatatgtgataaaatatgtttcttagacttttctgtttggtatattaattttttaaacattttctcctgaattctggtcgtagtcaccattactcttgagtgttaccctatactatactcggcctgcttggacctaaagcaataaatagtttagaaacgtgtgtgtgtgtgtgtgtgtgtgtgtgagagagagagagagagagagagagagagagagagagacagagacagagacagagacagagacagagagacacagagacaaagagagggagccttaaaataaaaaaaaacacagaaagaaaaggggagttgggggcagaaagataaggggagttggggtgtgaagatgaagaagagaaagcaaaaaggacacaccatgtggaagatcacagagtctctggaagtgtgactctggtgcttcctaaggaagttcaggcactgccactttagccttgagccatcccgactttctcttctctgcctttgcactctcttcctctgttggccatagctgtcaatttggcttaatacattttctggaaatttgtattggatgccaattttaaagtcagatttcacattaaaaaaatgaagacttgggctgttttacaaagtacttgccacagcagcttcttattccctaggtctgaaagtagtctgttctggccgccaggacaacttgaacagaactttattttctcacagttctgaggtttggaagtctaagatcaaagtgccaacttgtttcttcctgtagactgcctttggcttacaggccactgtatccttgctagacctttcccttatggcggccgcgtaccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaa

SEQ ID NO: 28 - protein, 745 aa, synthetic construct
  REGION 1..745: note = human FAP recombinant protein
GSHHHHHHGSLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 29 - protein, 746 aa, synthetic construct
  REGION 1..746: note = murine FAP recombinant protein
GSHHHHHHGSLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 30 - protein, 761 aa, Mus musculus
  REGION 1..761: note = murine fibroblast activation protein alpha (FAP)
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 31 - protein, 20 aa, Homo sapiens
  REGION 1..20: note = human Cystatin-S signal peptide
MARPLCTLLLLMATLAGALA

SEQ ID NO: 32 - protein, 134 aa, synthetic construct
  REGION 1..134: note = nontargeting control VHH R3B23
QVQLQESGGGSVQAGGSLRLSCAASGDTGYMGWFRQAPGKEREGVAVINSDSGVGSTYYADSVKGRFTISRDNAKNTVYLQMNSLKPEDTAIYYCAAGHFSDYVSPWTWREIYRYNVWGQGTQVTVSSHHHHHH

SEQ ID NO: 33 - DNA, 345 bp, synthetic construct
  misc_feature 1..345: note = nucleic acid encoding B1
  source 1..345: mol_type = other DNA
gatgtgcagctggtggagtctgggggaggcttggtgcagcctggggggtctctgagactctcctgtgtagcctctggaaggctctccagtagcaattccatggcctggtatcgccaggttccagggaagcgtcgcgagttggtcgcgggaattactggtggtggtgagacaaactatgcagacttcgtgggtggccgattcaccatctccagagacaacgccaagaacgggctgtatctgcaattgaacggcctgaaacctgaggacacggccgcctattattgtaatttctggcccccacttatcaactactggggccaagggacccaggtcaccgtctcctca

SEQ ID NO: 34 - DNA, 345 bp, synthetic construct
  misc_feature 1..345: note = nucleic acid encoding B2
  source 1..345: mol_type = other DNA
gatgtgcagctggtggagtctgggggaggcttggtgcaggctggggggtctctgagactctcctgtgcagtttctggaagcatctccagtgccaatagcatgggctggtaccgccaggctccagggaagcagcgcgacgtggtcgcaggtcttactactggtggtaggagtcactatgcagactccgtgaagggccgattcaccatctccagagacaatgccaagaacacggtgtatctgcaaatgaacagcctgaaagcagaggacacggccgtctattactgtaatttgtggccgccggttcagggctactggggccaggggacccaggtcaccgtctcctca

SEQ ID NO: 35 - DNA, 348 bp, synthetic construct
  misc_feature 1..348: note = nucleic acid encoding B3
  source 1..348: mol_type = other DNA
gatgtgcagctggtggagtctgggggaggcttggtgcaggctggggggtctctgagactctcctgtgcagtctctggaagactcttcagtaccaatgccatgggctggtaccgccaggctccagggaagcagcgcgagttggtcgcaggcattactggtggtgatagatcaaactatgcagactccgtgaagggccgattcaccatctccagagacaatggcaagaacacgctgtatctgcaaatgaacagcctgaaacctgaggacacggccgtctattactgtaatttctacccgcctattgtgggtgactactggggccaggggacccaggtcaccgtctcctca

SEQ ID NO: 36 - DNA, 348 bp, synthetic construct
  misc_feature 1..348: note = nucleic acid encoding B4
  source 1..348: mol_type = other DNA
gatgtgcagctggtggagtctgggggaggattggtgcaggttgggggctctctgagactctcctgtgtagcctctggattcaccttcagtagctactacatgagctgggtccgccaggctccagggaaggggctggagtgggtggccagtatttatgctgacggtgatatgacatactatgcagactccgtgaggggccgattcaccatctccagagacaacgccaagaacacgctgtatctgcaaatgaacagtctgaaatctgaggacacggccgtgtattactgtgcaaaagatcccctccccccctatcatgttaaccaggggacccaggtcaccgtctcctca

SEQ ID NO: 37 - protein, 25 aa, synthetic construct
  REGION 1..25: note = FR1 of B1
DVQLVESGGGLVQPGGSLRLSCVAS

SEQ ID NO: 38 - protein, 17 aa, synthetic construct
  REGION 1..17: note = FR2 of B1
MAWYRQVPGKRRELVAG

SEQ ID NO: 39 - protein, 38 aa, synthetic construct
  REGION 1..38: note = FR3 of B1
NYADFVGGRFTISRDNAKNGLYLQLNGLKPEDTAAYYC

SEQ ID NO: 40 - protein, 11 aa, synthetic construct
  REGION 1..11: note = FR4 of B1
WGQGTQVTVSS

SEQ ID NO: 41 - protein, 25 aa, synthetic construct
  REGION 1..25: note = FR1 of B2
DVQLVESGGGLVQAGGSLRLSCAVS

SEQ ID NO: 42 - protein, 17 aa, synthetic construct
  REGION 1..17: note = FR2 of B2
MGWYRQAPGKQRDVVAG

SEQ ID NO: 43 - protein, 38 aa, synthetic construct
  REGION 1..38: note = FR3 of B2
HYADSVKGRFTISRDNAKNTVYLQMNSLKAEDTAVYYC

SEQ ID NO: 44 - protein, 11 aa, synthetic construct
  REGION 1..11: note = FR4 of B2
WGQGTQVTVSS

SEQ ID NO: 45 - protein, 25 aa, synthetic construct
  REGION 1..25: note = FR1 of B2
DVQLVESGGGLVQAGGSLRLSCAVS

SEQ ID NO: 46 - protein, 17 aa, synthetic construct
  REGION 1..17: note = FR2 of B2
MGWYRQAPGKQRELVAG

SEQ ID NO: 47 - protein, 38 aa, synthetic construct
  REGION 1..38: note = FR3 of B2
NYADSVKGRFTISRDNGKNTLYLQMNSLKPEDTAVYYC

SEQ ID NO: 48 - protein, 11 aa, synthetic construct
  REGION 1..11: note = FR4 of B2
WGQGTQVTVSS

SEQ ID NO: 49 - protein, 25 aa, synthetic construct
  REGION 1..25: note = FR1 of B4
DVQLVESGGGLVQVGGSLRLSCVAS

SEQ ID NO: 50 - protein, 17 aa, synthetic construct
  REGION 1..17: note = FR2 of B4
MSWVRQAPGKGLEWVAS

SEQ ID NO: 51 - protein, 38 aa, synthetic construct
  REGION 1..38: note = FR3 of B4
YYADSVRGRFTISRDNAKNTLYLQMNSLKSEDTAVYYC

SEQ ID NO: 52 - protein, 11 aa, synthetic construct
  REGION 1..11: note = FR4 of B4
VNQGTQVTVSS

SEQ ID NO: 53 - protein, 9 aa, synthetic construct
  REGION 1..9: note = Exemplary HA tag
YPYDVPDYA

SEQ ID NO: 54 - protein, 10 aa, synthetic construct
  REGION 1..10: note = Exemplary HA tag
YPYDVPDYGS

SEQ ID NO: 55 - protein, 15 aa, synthetic construct
  REGION 1..15: note = Exemplary cysteine tag
SPSTPPTPSPSTPPC

(19) United States
(12) Patent Application Publication
(10) Pub. No.: US 2023/0381350 A1
(43) Pub. Date: Nov. 30, 2023

(54) ANTIBODY FRAGMENT AGAINST FAP

(71) Applicant: Precirix N.V., Brussels (BE)

(72) Inventors: Tony LAHOUTTE, Brussels (BE); Nick DEVOOGDT, Brussels (BE); Matthias D'HUYVETTER, Brussels (BE); Sam MASSA, Brussels (BE)

(21) Appl. No.: 18/182,034

(22) Filed: Mar. 10, 2023

Related U.S. Application Data
(63) Continuation of application No. PCT/EP21/75009, filed on Sep. 10, 2021.

(30) Foreign Application Priority Data
Sep. 10, 2020 (EP) 20195428.6

(51) Int. Cl. A61K 51/10; C07K 16/40; A61P 35/00; G01N 33/574

(52) U.S. Cl. CPC A61K 51/1075; C07K 16/40; A61P 35/00; G01N 33/57488; A61K 2039/505

(57) ABSTRACT

The present invention relates to the field of antibody fragments, which specifically binds an epitope of human and/or murine FAP and which may be linked to an entity such as a moiety. The antibody fragment and the compound formed may be used for therapy or diagnostic purposes.

### RELATED APPLICATIONS

This application is a continuation of PCT International Application PCT/EP2021/075009 (published as WO2022/053651), filed Sep. 10, 2021, which claims priority to EP Application No. 20195428.6, filed Sep. 10, 2020, the entirety of each of which is hereby incorporated by reference.

### SEQUENCE LISTING

The content of the electronically submitted Sequence Listing XML (SL_198676); Size: 98,304 bytes; Created: Mar. 9, 2023) is herein incorporated by reference in its entirety.

### FIELD OF THE INVENTION

The present invention relates to the field of antibody fragments, which specifically binds an epitope of Fibroblast Activation Protein (FAP) and which may be linked to an entity such as a moiety. Depending on the application of said antibody fragment, the moiety may be a label which may be a radionuclide.

The present invention relates to labelled antibody fragments for use in the prevention and/or treatment of cancer.

### BACKGROUND

Cancers figure among the leading causes of morbidity and mortality worldwide. There is a continuous need for improved therapies combatting cancer while minimizing side effects. FAP has been found to be expressed or even over-expressed on Cancer-Associated Fibroblasts (CAF) and also on cancer cells such as bone, brain, breast, colorectal, esophageal, gastric, liver, lung, ovarian, pancreatic, parathyroid, renal cancer cells (Puré et al 2018, Oncogene August; 37(32):4343-43573), while FAP is not expressed or to a low level on healthy cells. FAP therefore seems an interesting cancer target. So far no antibody targeting FAP had been approved as therapeutic antibody.

As such, there is a need in the art for further antibodies that target human FAP.

### LEGEND TO THE DRAWINGS

FIG. 1. Different elements of the targeting vector.

FIG. 2. Signal-to-background ratios of HA-His₆-tagged VHHs B1, B2, B3 and B4 binding to human FAP recombinant protein, murine FAP recombinant protein or human DPP IV recombinant protein, as determined by ELISA. *Not determined.

FIG. 3. Δmfi values of the binding of HA-His₆-tagged VHHs B1, B2, B3 and B4 to human FAP-expressing GM05389 cells and murine FAP-expressing HEK293 cells. The Δmfi values for VHH B3 on HEK-murine FAP cells and VHH B4 on GM05389 cells were smaller than 10.

FIG. 4. Substrate hydrolysis by human FAP recombinant protein, pre-incubated with the HA-His₆-tagged VHHs B1, B2 or B3, Talabostat mesylate (inhibitor control), or without a FAP-binding molecule (positive control). For every condition, the mean Relative Fluorescence Units (n=2) is shown in function of time.

FIG. 5. The cellular retention of ¹³¹I-labeled B1 (in its His₆-tagged and untagged version) on human FAP-expressing GM05389 cells over 24 hours. Data expressed as mean±SD (n=3).

FIG. 6. The cellular retention of ¹⁷⁷Lu-labeled untagged VHH B1 on human FAP-expressing GM05389 and HEK-293 cells over 24 hours. Data expressed as mean±SD (n=3).

FIG. 7. The cellular retention of ²²⁵Ac-labeled untagged VHH B1 on human FAP-expressing GM05389 cells over 24 hours. Data expressed as mean±SD (n=3).

FIG. 8A-FIG. 8C: The Kaplan-Meier curves describing the survival of mice treated with different radiolabeled untagged VHH B1. FIG. 8A: ¹³¹I-labeled untagged VHH B1 is effective in mice with established subcutaneous human glioblastoma tumors (U87 MG) as revealed by its capacity to inhibit tumor development and the resulting extended survival of treated mice; FIG. 8B: ²²⁵Ac-labeled untagged VHH B1 is effective in mice with established subcutaneous human HEK-293 tumors as revealed by its capacity to inhibit tumor development and the resulting extended survival of treated mice; FIG. 8C: ¹⁷⁷Lu-labeled untagged VHH B1 is effective in mice with established subcutaneous human glioblastoma tumors (U87 MG) as revealed by its capacity to inhibit tumor development and the resulting extended survival of treated mice. MS: Mean survival.

### DETAILED DESCRIPTION OF THE INVENTION

Antibody Fragment

In a first aspect of the invention, there is provided an antibody fragment which specifically binds human and/or murine FAP. In an embodiment, said antibody fragment is represented by an amino acid sequence that comprises an amino acid sequence having at least 80% sequence identity with at least one of SEQ ID NO:1, 2, 3, 4 or a portion thereof.

In an embodiment, the antibody fragment provided, which specifically binds human and/or murine FAP, fulfils at least one of the following:
a. the epitope is comprised within amino acid 26 to 760 of SEQ ID NO:26, preferably the epitope is comprised within (or comprises) amino acids 65-90 and/or 101-140 of SEQ ID NO:26,
b. wherein at least amino acids I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and/or Y458 of SEQ ID NO:26 interacts with said antibody fragment,
c. said antibody fragment is represented by an amino acid sequence that comprises an amino acid sequence having at least 80% sequence identity with at least one of SEQ ID NO:1, 2, 3, 4 or a portion thereof.

In an embodiment of this aspect, the antibody fragment provided which specifically binds human and/or murine FAP fulfils at least one of the following:
a. the epitope is comprised within amino acids 26 to 760 of SEQ ID NO:26, preferably the epitope is comprised within (or comprises) amino acids 65-90 and/or 101-140 of SEQ ID NO:26,
b. the antibody fragment specifically binds to the following amino acids of SEQ ID NO:26:
   1) at least one, or at least two or at least three amino acids selected from: I62, S63, G64, Q65, E66, and/or
   2) at least one, or at least two or at least three amino acids selected from: I76, V77, L78, Y79, N80, I81, E82, T83, G84, Q85, S86, Y87, T88, I89, L90, S91, and/or
   3) at least one, or at least two or at least three amino acids selected from L105, S106, P107, D108, R109, Q110, F111, and/or
   4) at least one, or at least two or at least three amino acids selected from D134, L135, S136, N137, and/or
   5) V158 and/or G159 and/or
   6) R175, and/or
   7) D457 and/or Y458,
c. said antibody fragment is represented by an amino acid sequence that comprises an amino acid sequence having at least 80% sequence identity with at least one of SEQ ID NO:1, 2, 3, 4 or a portion thereof.

In an embodiment of this aspect, the antibody fragment provided which specifically binds human and/or murine FAP, which has its epitope comprised within amino acids 26 to 760 of SEQ ID NO:26, specifically binds to the following amino acids of SEQ ID NO:26:
1) at least one, or at least two or at least three amino acids selected from: I62, S63, G64, Q65, E66, and
2) at least one, or at least two or at least three amino acids selected from: I76, V77, L78, Y79, N80, I81, E82, T83, G84, Q85, S86, Y87, T88, I89, L90, S91, and
3) at least one, or at least two or at least three amino acids selected from L105, S106, P107, D108, R109, Q110, F111, and
4) at least one, or at least two or at least three amino acids selected from D134, L135, S136, N137, and
5) V158 and/or G159 and
6) R175, and
7) D457 and/or Y458.

In an embodiment of this aspect, the antibody fragment provided, which specifically binds human and/or murine FAP, which has its epitope comprised within (or has its epitope which comprises) amino acids 65-90 and/or 101-140 of SEQ ID NO:26, specifically binds to the following amino acids of SEQ ID NO:26:
1) at least one, or at least two or at least three amino acids selected from: I62, S63, G64, Q65, E66, and
2) at least one, or at least two or at least three amino acids selected from: I76, V77, L78, Y79, N80, I81, E82, T83, G84, Q85, S86, Y87, T88, I89, L90, S91, and
3) at least one, or at least two or at least three amino acids selected from L105, S106, P107, D108, R109, Q110, F111, and
4) at least one, or at least two or at least three amino acids selected from D134, L135, S136, N137, and
5) V158 and/or G159 and
6) R175, and
7) D457 and/or Y458.

Throughout the application, FAP is synonymous with FAPalpha and corresponds to the polypeptide Prolyl endopeptidase FAP, which is also named Fibroblast activation protein alpha (FAPalpha). The gene encoding this protein is called FAP.

TABLE 1
Amino acid sequence of human and murine FAP (derived from Uniprot entry
Q12884 and P97321, respectively)

| | |
|---|---|
| SEQ ID NO: 26 | MKTWVKIVFGVATSAVLALLVMCIVLRPSRVHNSEENTMRALTLKDILNG |
| Human | TFSYKTFFPNWISGQEYLHQSADNNIVLYNIETGQSYTILSNRTMKSVNA |
| | SNYGLSPDRQFVYLESDYSKLWRYSYTATYYIYDLSNGEFVRGNELPRP |
| | IQYLCWSPVGSKLAYVYQNNIYLKQRPGDPPFQITFNGRENKIFNGIPDW |
| | VYEEEMLATKYALWWSPNGKFLAYAEFNDTDIPVIAYSYYGDEQYPRTI |
| | NIPYPKAGAKNPVVRIFIIDTTYPAYVGPQEVPVPAMIASSDYYFSWLTW |
| | VTDERVCLQWLKRVQNVSVLSICDFREDWQTWDCPKTQEHIEESRTG |
| | WAGGFFVSTPVFSYDAISYYKIFSDKDGYKHIHYIKDTVENAIQITSGKWE |
| | AINIFRVTQDSLFYSSNEFEEYPGRRNIYRISIGSYPPSKKCVTCHLRKER |
| | CQYYTASFSDYAKYYALVCYGPGIPISTLHDGRTDQEIKILEENKELENAL |
| | KNIQLPKEEIKKLEVDEITLWYKMILPPQFDRSKKYPLLIQVYGGPCSQSV |
| | RSVFAVNWISYLASKEGMVIALVDGRGTAFQGDKLLYAVYRKLGVYEVE |
| | DQITAVRKFIEMGFIDEKRIAIWGWSYGGYVSSLALASGTGLFKCGIAVA |
| | PVSSWEYYASVYTERFMGLPTKDDNLEHYKNSTVMARAEYFRNVDYLLI |
| | HGTADDNVHFQNSAQIAKALVNAQVDFQAMWYSDQNHGLSGLSTNHL |
| | YTHMTHFLKQCFSLSD |
| SEQ ID NO: 30 | MKTWLKTVFGVTTLAALALVVICIVLRPSRVYKPEGNTKRALTLKDILNGT |
| Murine | FSYKTYFPNWISEQEYLHQSEDDNIVFYNIETRESYIILSNSTMKSVNATD |
| | YGLSPDRQFVYLESDYSKLWRYSYTATYYIYDLQNGEFVRGYELPRPIQ |
| | YLCWSPVGSKLAYVYQNNIYLKQRPGDPPFQITYTGRENRIFNGIPDWV |
| | YEEEMLATKYALWWSPDGKFLAYVEFNDSDIPIIAYSYYGDGQYPRTINI |
| | PYPKAGAKNPVVRVFIVDTTYPHHVGPMEVPVPEMIASSDYYFSWLTW |
| | VSSERVCLQWLKRVQNVSVLSICDFREDWHAWECPKNQEHVEESRTG |
| | WAGGFFVSTPAFSQDATSYYKIFSDKDGYKHIHYIKDTVENAIQITSGKW |
| | EAIYIFRVTQDSLFYSSNEFEGYPGRRNIYRISIGNSPPSKKCVTCHLRKE |
| | RCQYYTASFSYKAKYYALVCYGPGLPISTLHDGRTDQEIQVLEENKELE |
| | NSLRNIQLPKVEIKKLKDGGLTFWYKMILPPQFDRSKKYPLLIQVYGGPC |
| | SQSVKSVFAVNWITYLASKEGIVIALVDGRGTAFQGDKFLHAVYRKLGVY |
| | EVEDQLTAVRKFIEMGFIDEERIAIWGWSYGGYVSSLALASGTGLFKCGI |
| | AVAPVSSWEYYASIYSERFMGLPTKDDNLEHYKNSTVMARAEYFRNVD |
| | YLLIHGTADDNVHFQNSAQIAKALVNAQVDFQAMWYSDQNHGISSGRS |
| | QNHLYTHMTHFLKQCFSLSD |

Within the context of the invention, the term “antibody fragment” refers to any fragment of an antibody or immunoglobulin. In an embodiment, the antibody fragment is a single-domain antibody fragment. In an embodiment, the antibody fragment is a heavy chain variable domain derived from a heavy chain antibody (VHH) or a fragment thereof. In a preferred embodiment, a single-domain antibody fragment is a VHH or a fragment thereof: the heavy chain variable domains derived from heavy chain antibodies (i.e. the VHH's) as disclosed herein consist of a single polypeptide chain. Within the context of the application, the expression “antibody fragment” may be replaced by “single-domain antibody fragment” or by “VHH” or by “a fragment of a VHH” or by “a functional fragment of a VHH”. Preferably a fragment of an antibody or of a VHH is a functional fragment as it exhibits at least an activity of the antibody or of the VHH to some extent. “Some extent” may mean at least 40%, 50%, 60%, 70%, 80%, 90%, 95%, 100% or more. A preferred activity of the antibody fragment, VHH or a fragment of a VHH is the specific binding to human and/or murine FAP. The “specific binding to human and/or murine FAP” has been defined later herein.

At the end of the description, a more detailed definition of “antibody”, “antibody fragment”, “agonist” “antagonist”, “variants of antibody fragment” is provided.

More particularly, the VHH's or fragments thereof disclosed herein are derived from an innate or adaptive immune system, preferably from a protein of an innate or adaptive immune system. Still more particularly, the VHH's disclosed herein may comprise 4 framework regions (FR) and 3 complementary determining regions (CDR), or any suitable fragment thereof (which will then usually contain at least some of the amino acid residues that form at least one of the CDR). In particular, the VHH's disclosed herein are easy to produce at high yield, preferably in a microbial recombinant expression system, and convenient to isolate and/or purify subsequently.

According to particular embodiments described in more details later herein, the invention provides an antibody fragment particularly suited for binding to human and/or murine FAP. In an embodiment, the antibody fragment of the invention specifically binds human and murine FAP. In an embodiment, the antibody fragment binds part of the extracellular domain of human and/or murine FAP.

Human FAP is quite attractive to be targeted as it is specifically expressed and more specifically overexpressed in cancer-associated fibroblasts (CAF) which have a tumorigenic function (Puré et al 2018, Oncogene August; 37(32):4343-4357). It is also expressed in some cancer cells (such as leukemia, bone, uterus, pancreas, skin, muscle, brain, breast, colorectal, esophageal, gastric, liver, lung, ovarian, parathyroid, renal cancer as disclosed later herein) and poorly expressed in healthy cells. Human FAP may therefore be considered as a tumour antigen or a cancer cell antigen and may therefore be used as diagnostic and/or therapeutic target.

However other applications (diagnostic and therapeutic) of the antibody fragment of the invention are also encompassed by the present invention. Such other diagnostic and/or therapeutic applications are not linked to cancer but may be linked to fibrosis, wound healing, myocardial infarction, atherosclerosis, arthritis and other inflammatory and fibrotic diseases. In other words, the antibody fragment of the invention may be used in a diagnostic and/or therapeutic application to diagnose and/or treat fibrosis, wound healing, myocardial infarction, atherosclerosis, arthritis and other inflammatory and fibrotic diseases. More detailed explanation is given later herein.

The antibody fragment of the invention may comprise CDR (complementarity determining regions) sequences of antibodies (or may be based on and/or derived from such CDR sequences, as further described herein), they will also generally be referred to herein as ‘CDR sequences’ (i.e. as CDR1 sequences, CDR2 sequences and CDR3 sequences, respectively). In an embodiment, the VHH's as disclosed herein comprise at least one amino acid sequence that is chosen from the group consisting of the CDR1 sequences, CDR2 sequences and CDR3 sequences that are described herein. Thus, in particular embodiments, the present invention provides heavy chain variable domains derived from heavy chain antibodies with the (general) structure:
FR1-CDR1-FR2-CDR2-FR3-CDR3-FR4

Within the context of the invention the IMGT nomenclature is used to define the FR (framework regions) FR1, FR2, FR3 and FR4 and corresponding CDR regions CDR1, CDR2, and CDR3. The definition of the IMGT nomenclature used is provided later herein in the general part dedicated to the definition of the invention. It should however be noted that the invention in its broadest sense is not limited to a specific structural role or function that these stretches of amino acid residues may have in the heavy chain variable domains as disclosed herein, as long as these stretches of amino acid residues allow the variable domains as disclosed herein to specifically bind to human and/or murine FAP. Thus, generally, the invention in its broadest sense relates to an antibody fragment, such as a single-domain antibody fragment, preferably a VHH or a fragment thereof, which can be coupled to an entity such as a moiety. Within the context of the invention, an antibody fragment, preferably a VHH or a fragment thereof coupled to an entity such as a moiety may be called a compound.

This moiety may be a label. The label may be a radionuclide. Alternatively, the label may be non-radioactive. When the compound of the invention comprises a label, it may be called a labelled compound. A radioactive labelled compound is preferably used for treating a cancer associated with the expression of human FAP in a CAF cell and/or in a cancer cell. A non-radioactive labelled compound is preferably used in a diagnostic application as later disclosed herein.

In another application, the moiety may be a medicament to treat fibrosis, wound healing, myocardial infarction, atherosclerosis, arthritis and other inflammatory and fibrotic diseases. In such applications, the antibody fragment of the invention is used to target the medicament to the site of the disease listed in order to treat it.

In a preferred embodiment, the moiety is a prodrug of adrenomedullin as described in WO2013064508A1, an autotaxin inhibitor as described in WO2014097151A2, a pyrimido[4,5-b]quinoline-4,5 (3h,10h)-dione derivative as described in WO2014091446A1, an amiloride derivative as described in WO2013064450A1, a pyrrolo[2,3-d]pyrimidine derivative as described in WO2014177527A1, a pyrazolopyridine derivative or a pyrazolopyrimidine derivative as described in WO2015173683A1, a piperidino-dihydrothienopyrimidine sulfoxide derivative as described in WO201326797A1, a 2-[pyridin-3-yl]-2,3-dihydro-benzo[1,4]dioxine derivative as described in WO2016061161A1, a pyridine derivative as described in WO201486705A1, a pyridine derivative or a pyrazine derivative as described in WO2011113894A1, an aminopyrimidinyl derivative as described in WO201627195A1, a carboxamide derivative as described in WO2015175796A1, an oxazole substituted indazole derivative as described in WO2010125082A1, a bisphenyl butanoic phosphonic acid derivative as described in WO2014126979A1, a pyrazine derivative as described in WO201235158A1, an oxazolidin-2-one-pyrimidine derivative as described in WO201472956A1, a pyrazolopyridinamine derivative as described in WO201696721A1, a N-(hetero)aryl,2-(hetero)aryl-substituted acetamide derivative as described in WO2010101849A1, a 3-azabicyclo[3.1.0]hexane derivative as described in WO2017115205A1, a phenoxyacetamide derivative as described in WO201728927A1, a N-(5-(4-acetylpiperazin-1-yl)pyridin-2-yl)-2-(2′-fluoro-3-methyl-2,4′-bipyridin-5-yl) acetamide derivative or a 2-(2′,3-dimethyl-2,4′-bipyridin-5-yl)-N-(5-(pyrazin-2-yl)pyridin-2-yl)acetamide derivative as described in WO2017221142A1, a xanthine derivative as described in WO2013171166A1, a xanthine derivative as described in WO2013174768A1, a heterocyclylmethyl-thienouracile derivative as described in WO2016150901A1, an n,2-diarylquinoline-4-carboxamide derivative as described in WO201637954A1, a pyridin-2-amide derivative as described in WO2012168350A1, a benzamide derivative acting as modulator of cellular adhesion as described in WO2005044817A1, a 3-amino-pyridine derivative as described in WO2012117000A1, a Nampt or rock inhibitor as described in WO201267965A1, a oxetane derivative as described in WO201616242A1, a 1,1,1-trifluoro-3-hydroxypropan-2-yl carbamate derivative as described in WO2018134695A1, a pyrazol derivative as described in WO2014135507A1, an indole derivative as described in WO2009156462A1, a [1,2,31triazolo[4,5-d]pyrimidine derivative as described in WO201815088A1, an sgc stimulator as described in WO2016177660A1, a CFTR protein as described in WO201760879A1, a 6-carboxylic acid of a benzimidazole or of a 4-aza-, 5-aza-, 7-aza- or 4,7-diaza-benzimidazole as described in WO2018109607A1, a benzamide derivative as described in WO201728926A1, an 8-azabicyclo [3.2.1] octane derivative as described in WO201287519A1, a triazolo[4,5-d]pyrimidine derivative as described in WO201671375A1, an aryl sultam derivative as described in WO2015104354A1, a 2-(azaindol-2-yl)benzimidazole derivative as described in WO201415905A1, or a quinuclidine or a iso-quinuclidine derivative as described in WO2005104745A1. Preferably, a compound according to this embodiment is a medicament to treat fibrosis, wound healing, myocardial infarction, atherosclerosis, arthritis and/or other inflammatory and fibrotic diseases.

WO2013064508A1, WO2014097151A2, WO2014091446A1, WO2013064450A1, WO2014177527A1, WO2015173683A1, WO201326797A1, WO2016061161A1, WO201486705A1, WO2011113894A1, WO201627195A1, WO2015175796A1, WO2010125082A1, WO2014126979A1, WO201235158A1, WO201472956A1, WO201696721A1, WO2010101849A1, WO2017115205A1, WO201728927A1, WO2017221142A1, WO2013171166A1, WO2013174768A1, WO2016150901A1, WO201637954A1, WO2012168350A1, WO2005044817A1, WO2012117000A1, WO201267965A1, WO201616242A1, WO2018134695A1, WO2014135507A1, WO2009156462A1, WO201815088A1, WO2016177660A1, WO201760879A1, WO2018109607A1, WO201728926A1, WO201287519A1, WO201671375A1, WO2015104354A1, WO201415905A1 and WO2005104745A1 are incorporated in their entirety, and all compounds disclosed therein may be a moiety linked to the antibody fragment in the context of the current application.

The antibody fragment such as a single-domain antibody fragment, preferably a VHH or fragment thereof may be characterized by a functional feature and/or by a structural feature. Examples of structural features are sequence related and examples of functional features are related to an activity of said antibody fragment. An activity may be a specific binding activity. An activity may also be the absence of a specific binding activity or the absence of the detection of a specific binding activity.

Specific binding of an antibody fragment can be determined in any suitable manner known per se, including for example biopanning, Scatchard analysis and/or competitive binding assays, such as radioimmunoassays (RIA), enzyme immunoassays (EIA) (also called Enzyme-Linked Immuno Sorbent Assay, ELISA), sandwich competition assays, Surface Plasmon Resonance (SPR), or Bio-Layer Interferometry and the different variants thereof known in the art. Each of these assays may be carried out in vitro using the human and/or murine FAP recombinant protein which may be immobilised on a support or in solution. Alternatively, under some specific circumstances, some of these assays may be carried out in vitro using cells that express human and/or murine FAP. Such cells may endogenously express or overexpress human and/or murine FAP. The assessment is usually carried out in vitro in a culture medium or in PBS or in a suitable medium or buffer. A preferred cell is a fibroblast cell expressing human and/or murine FAP. A preferred cell line may be GM05389 or U-87 MG. Alternatively a preferred cell is a transfected cell expressing human and/or murine FAP. A preferred transfected cell line may be HEK293.

Alternatively, the binding of the antibody fragment may be assessed in vivo in an animal expressing human and/or murine FAP. In such setting, the antibody fragment is preferably labelled, more preferably radiolabelled and an imaging technique is used. A preferred imaging technique is SPECT/CT, PET/CT, SPECT/MRI or PET/MRI. Cells overexpressing human and/or murine FAP may also be xenografted into the animal. It is also possible to use the human FAP knock-in mouse of the invention expressing human FAP.

The wording “in vitro” is therefore used herein in the context of a cell-free assay when the human and/or murine FAP recombinant protein is immobilized on a support or in solution, or in the context of a cell in culture. As opposed to that, the wording “in vivo” or “ex vivo” is used herein in the context of a non-human animal or a tissue or organ of this non-human animal. Usually “ex vivo” is used when a quantification is carried out on a tissue or organ of a non-human or human animal and “in vivo” is used when a quantification via an imaging method is carried out on a non-human or human animal. Usually “binding” is assessed using in vitro conditions and is further confirmed using in vivo conditions.

In the in vitro, in vivo and ex vivo assays disclosed herein, it is preferred to use a negative control. It is also possible to assess the specific binding to human and/or murine FAP in the presence of other antigens as explained later herein. In the experimental part, several assays have been used to assess the specific binding of the antibody fragment of the invention: example 4 wherein ELISA and bio-layer interferometry have been used, example 5 wherein the binding has been assessed in the human FAP knock-in mouse using SPECT/CT imaging and/or ex vivo gamma counting of dissected tissues, and example 6 wherein the binding has been assessed in a mouse comprising of cells overexpressing human FAP using SPECT/CT imaging and/or ex vivo gamma counting of dissected tissues.

The term “affinity”, “specific binding”, “binding”, “binding activity” or “specific binding activity”, as used herein, refers to the degree to which an antibody fragment such as a single-domain antibody fragment preferably a VHH, or a fragment thereof binds to human and/or murine FAP so as to shift the equilibrium of human and/or murine FAP and the antibody fragment towards the presence of a complex formed by their binding. The binding may be assessed using SPR or bio-layer interferometry. Thus, for example, where human and/or murine FAP and the antibody fragment are combined in relatively equal concentrations, the antibody fragment of high affinity will bind to the available human and/or murine FAP so as to shift the equilibrium towards high concentrations of the resulting complex. The equilibrium dissociation constant (K_{D}) is commonly used to describe the affinity between the protein binding domain (antibody fragment) and the antigenic target (human and/or murine FAP). Typically, the equilibrium dissociation constant is less than 10⁻⁷ M. Preferably, the equilibrium dissociation constant is less than 10⁻⁸ M, or less than 10⁻⁹M, or more preferably, ranging from 10⁻⁹ M and 10⁻¹² M.

Any antibody fragment as disclosed herein is preferably such that it specifically binds (as defined herein) to human and/or murine FAP with an equilibrium dissociation constant (K_{D}) ranging from 10⁻⁹ to 10⁻¹² moles/liter or from 10⁻¹⁰ to 10⁻¹² moles/liter, preferably assessed using bio-layer interferometry.

The ‘specificity’ of an antibody fragment such as a single-domain antibody fragment, preferably a VHH, or fragments thereof as disclosed herein can be determined based on affinity and/or avidity. The ‘affinity’ of an antibody fragment as disclosed herein is represented by the equilibrium constant for the dissociation of the antibody fragment as disclosed herein and human and/or murine FAP to which it binds. The lower the K_{D} value, the stronger the binding strength between the antibody fragment as disclosed herein and the target protein of interest to which it binds. Alternatively, the affinity can also be expressed in terms of the equilibrium association constant (K_{A}), which corresponds to 1/K_{D}. The binding affinity of an antibody fragment as disclosed herein can be determined in a manner known to the skilled person, depending on the specific target protein of interest. The ‘avidity’ of an antibody fragment as disclosed herein is the measure of the strength of binding between the antibody fragment as disclosed herein and the pertinent target protein of interest. Avidity is related to both the affinity between a binding site on the target protein of interest and a binding site on the antibody fragment as disclosed herein and the number of pertinent binding sites present on the antibody fragment as disclosed herein. Preferred antibody fragments of the invention such as VHHs have only one single-domain and therefore only one single binding site. The affinity exhibited by such a single-domain antibody fragment is in the subnanomolar range and is therefore quite exceptional in view of the presence of a single binding site. A K_{D} value greater than about 1 millimolar is generally considered to indicate non-binding or non-specific binding. It is generally known in the art that the K_{D} can also be expressed as the ratio of the dissociation rate constant of a complex, denoted as k_{off} or k_{d} (expressed in seconds⁻¹ or s⁻¹), to the rate constant of its association, denoted kₒₙ or kₐ (expressed in molar⁻¹ seconds⁻¹ or M⁻¹ s⁻¹). In particular, the antibody fragment as disclosed herein will bind to the target protein of interest (i.e. human and/or murine FAP) with a k_{off} ranging from 0.1 and 0.00001 s⁻¹, or ranging from 10⁻² to 10⁻⁵ s⁻¹ and/or a kₒₙ ranging from 1,000 and 10,000,000 M⁻¹ s⁻¹ or ranging from 10⁴ to 10⁷ M⁻¹ s⁻¹ or from 10⁵ to 10⁷ M⁻¹ s⁻¹. Binding affinities, k_{off} and kₒₙ rates may be determined by means of methods known to the person skilled in the art, for example ELISA methods, isothermal titration calorimetry, SPR, bio-layer interferometry, fluorescence-activated cell sorting analysis, and the more (see example 4).

In a preferred embodiment, the antibody fragment as disclosed herein specifically binds to human and/or murine FAP with a k_{off} ranging from 0.1 and 0.00001 s⁻¹, or ranging from 10⁻² to 10⁻⁵ s⁻¹ or from 10³ to 10⁻⁵ s⁻¹, preferably assessed using bio-layer interferometry.

In a preferred embodiment, an antibody fragment as disclosed herein is such that it specifically binds (as defined herein) to human and/or murine FAP with a K_{D} ranged from 10⁻⁹ to 10⁻¹² moles/liter and/or a k_{off} ranging from 10⁻² to 10⁻⁵ s⁻¹ preferably assessed using bio-layer interferometry, more preferably with a K_{D} ranged from 10⁻⁹ to 10⁻¹² moles/liter and a k_{off} ranging from 10⁻² to 10⁻⁵ s⁻¹.

Accordingly, an antibody fragment such as a single-domain antibody fragment (preferably a VHH or a fragment thereof), as disclosed herein is said to ‘specifically bind to’ human and/or murine FAP when that antibody fragment has affinity for, specificity for and/or is specifically directed against that target (or for at least one part or fragment thereof).

In respect of the antibody fragment such as a VHH or fragments thereof, as disclosed herein, the terms ‘binding region’, ‘binding site’ or ‘interaction site’ present on the antibody fragment as disclosed herein shall herein have the meaning of a particular site, part, locus, domain or stretch of amino acid residues present on the antibody fragment as disclosed herein that is responsible for binding or specific binding to human and/or murine FAP. This binding region present on the antibody fragment is called a paratope. Such binding region comprises, consists or essentially consists of specific amino acid residues from the amino acid sequence as disclosed herein of the antibody fragment which are in contact with human and/or murine FAP. The region or part or discrete amino acids of the extracellular domain of the human and/or murine FAP that is in contact with said antibody fragment may be called an epitope and are defined later herein. In an embodiment, the family of antibody fragments of the invention shares an epitope as defined later herein (second structural feature). This family of antibody fragments exhibits attractive properties both in vitro and in vivo. In vitro attractive properties may at least relate to their binding affinity and kinetics, or not modulating the FAP enzymatic activity. In vivo attractive properties may at least relate to their biodistribution and tumor targeting when said antibody fragment is present in the labelled compound of the invention.

The terms ‘specifically bind’ and ‘specific binding’, as used herein, generally refers to the ability of a polypeptide, in particular an immunoglobulin, such as an antibody, or an antibody fragment, such as a single-domain antibody fragment preferably a VHH or fragments thereof, to preferentially bind to a particular antigen such as human and/or murine FAP. Such an antibody fragment may also be identified as an antibody fragment raised against human and/or murine FAP.

The binding to human and/or murine FAP may be assessed in a homogeneous mixture of different antigens. In certain embodiments, a specific binding interaction will discriminate between desirable and undesirable antigens in a sample, in some embodiments more than about 10 to 100-fold or more (e.g., more than about 1000- or 10,000-fold).

In an embodiment, the binding may be assessed in vitro using cells expressing human and/or murine FAP, and optionally in vivo or ex vivo as earlier defined herein. These cells may be human cells and expressing endogenous human and/or murine FAP. Alternatively, these cells may overexpress human and/or murine FAP. Cells overexpressing human and/or murine FAP may be human or non-human cells. Preferred cells are fibroblast cells expressing human and/or murine FAP. A preferred transfected cell line is HEK293. A preferred cell line expressing FAP is GM05389 or U-87 MG. A preferred cancer cell line expressing human FAP is U-87 MG.

In an embodiment, the antibody fragment such as single-domain antibody fragment, preferably a VHH or fragments thereof, specifically binds to human and/or murine FAP. This assessment is preferably carried out using ELISA, Surface Plasmon Resonance or Bio-Layer Interferometry.

It is also expected that the antibody fragment such as single-domain antibody fragment, preferably the VHH or a fragment thereof of the invention will bind to a number of naturally occurring or synthetic analogs, variants, mutants, alleles, parts and fragments of human and/or murine FAP.

In an embodiment, the antibody fragment, preferably the VHH or a fragment thereof of the invention will specifically bind to at least those analogs, variants, mutants, alleles, naturally occurring, synthetic analogs, parts and fragments of human and/or murine FAP that (still) contain the epitope of the (natural/wild-type) antigen to which the antibody fragment binds.

The epitope of human FAP of the antibody fragment of the invention is comprised within amino acids 26 to 760 of SEQ ID NO:26.

In an embodiment, the epitope of the antibody fragment of the invention is comprised within amino acids 65-90 and/or 101-140 of SEQ ID NO:26.

In an embodiment, the epitope of the antibody fragment of the invention comprises the amino acid stretch or region 65-90 and/or 101-140 of SEQ ID NO:26.

In an embodiment, the epitope of the antibody fragment of the invention comprises the combination of amino acid stretch or region 65-90 and 101-140 of SEQ ID NO:26.

In an embodiment, the epitope of the antibody fragment of the invention is comprised within the combination of amino acid stretch or region 65-90 and 101-140 of SEQ ID NO:26.

In an embodiment, at least one of the following amino acids of SEQ ID NO:26 is bound or contacted or interacts with the antibody fragment: I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and Y458.

In an embodiment, the antibody fragment specifically binds to the following amino acids of SEQ ID NO:26:
1) at least one, or at least two or at least three amino acids selected from: I62, S63, G64, Q65, E66, and/or
2) at least one, or at least two or at least three amino acids selected from: I76, V77, L78, Y79, N80, I81, E82, T83, G84, Q85, S86, Y87, T88, I89, L90, S91, and/or
3) at least one, or at least two or at least three amino acids selected from L105, S106, P107, D108, R109, Q110, F111, and/or
4) at least one, or at least two or at least three amino acids selected from D134, L135, S136, N137, and/or
5) V158 and/or G159 and/or
6) R175, and/or
7) D457 and/or Y458.

In an embodiment, the antibody fragment specifically binds to the following amino acids of SEQ ID NO:26:
1) at least one, or at least two or at least three amino acids selected from: I62, S63, G64, Q65, E66, and
2) at least one, or at least two or at least three amino acids selected from: I76, V77, L78, Y79, N80, I81, E82, T83, G84, Q85, S86, Y87, T88, I89, L90, S91, and
3) at least one, or at least two or at least three amino acids selected from L105, S106, P107, D108, R109, Q110, F111, and
4) at least one, or at least two or at least three amino acids selected from D134, L135, S136, N137, and
5) V158 and/or G159 and
6) R175, and
7) D457 and/or Y458.

In an embodiment, the antibody fragment specifically binds to the following amino acids of SEQ ID NO:26:
1) at least two or at least three amino acids selected from: I62, S63, G64, Q65, E66, and/or
2) at least two or at least three amino acids selected from: I76, V77, L78, Y79, N80, I81, E82, T83, G84, Q85, S86, Y87, T88, I89, L90, S91, and/or
3) at least two or at least three amino acids selected from L105, S106, P107, D108, R109, Q110, F111, and/or
4) at least two or at least three amino acids selected from D134, L135, S136, N137, and/or
5) V158 and G159 and/or
6) R175, and/or
7) D457 and Y458.

In an embodiment, the antibody fragment specifically binds to the following amino acids of SEQ ID NO:26:
1) at least two or at least three amino acids selected from: I62, S63, G64, Q65, E66, and
2) at least two or at least three amino acids selected from: I76, V77, L78, Y79, N80, I81, E82, T83, G84, Q85, S86, Y87, T88, I89, L90, S91, and
3) at least two or at least three amino acids selected from L105, S106, P107, D108, R109, Q110, F111, and
4) at least two or at least three amino acids selected from D134, L135, S136, N137, and
5) V158 and G159 and
6) R175, and
7) D457 and Y458.

In an embodiment, the antibody fragment specifically binds to the following amino acids of SEQ ID NO:26:
1) at least three amino acids selected from: I62, S63, G64, Q65, E66, and/or
2) at least three amino acids selected from: I76, V77, L78, Y79, N80, I81, E82, T83, G84, Q85, S86, Y87, T88, I89, L90, S91, and/or
3) at least three amino acids selected from L105, S106, P107, D108, R109, Q110, F111, and/or
4) at least three amino acids selected from D134, L135, S136, N137, and/or
5) V158 and G159 and/or
6) R175, and
7) D457 and Y458.

In an embodiment, the antibody fragment specifically binds to the following amino acids of SEQ ID NO:26:
1) at least three amino acids selected from: I62, S63, G64, Q65, E66, and
2) at least three amino acids selected from: I76, V77, L78, Y79, N80, I81, E82, T83, G84, Q85, S86, Y87, T88, I89, L90, S91, and
3) at least three amino acids selected from L105, S106, P107, D108, R109, Q110, F111, and 4) at least three amino acids selected from D134, L135, S136, N137, and
5) V158 and G159 and
6) R175, and
7) D457 and Y458.

In an embodiment, the following amino acids of SEQ ID NO:26 are bound or contacted or interacted with the antibody fragment: I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and Y458. In this context, an amino acid of human of FAP may be bound by the antibody fragment of the invention when said amino acid belongs to the epitope of the antibody fragment.

An antibody fragment such as a single-domain antibody fragment preferably a VHH or fragments thereof, as disclosed herein is said to be ‘specific’ for a first target antigen of interest (i.e. human and/or murine FAP) as opposed to a second molecule, such as one of the closest homologues of FAP (i.e. DPP IV, Dipeptidyl amino-peptidase IV, Juillerat-Jeanneret, L., et al (2017), Expert Opinion on therapeutic targets, 21: 977-991) when it binds to the first target antigen of interest with an affinity that is at least 5 times, such as at least 10 times, such as at least 100 times, and preferably at least 1000 times higher than the affinity with which that antibody fragment as disclosed herein binds to the second molecule.

The amino acid sequence of DPP IV has 52% identity with the amino acid sequence of FAP (see example 4b) and still the antibody fragment of the invention can distinguish between the two related prolyl-specific serine proteases. Accordingly, in certain embodiments, when an antibody fragment as disclosed herein is said to be ‘specific for’ a first target antigen of interest as opposed to a second molecule, it may specifically bind to (as defined herein) the first target antigen of interest, but not to the second molecule. Within the context of the invention, an antibody fragment specifically binds an epitope of human and/or murine FAP and it does neither specifically bind DPP IV. This has been demonstrated in example 4b.

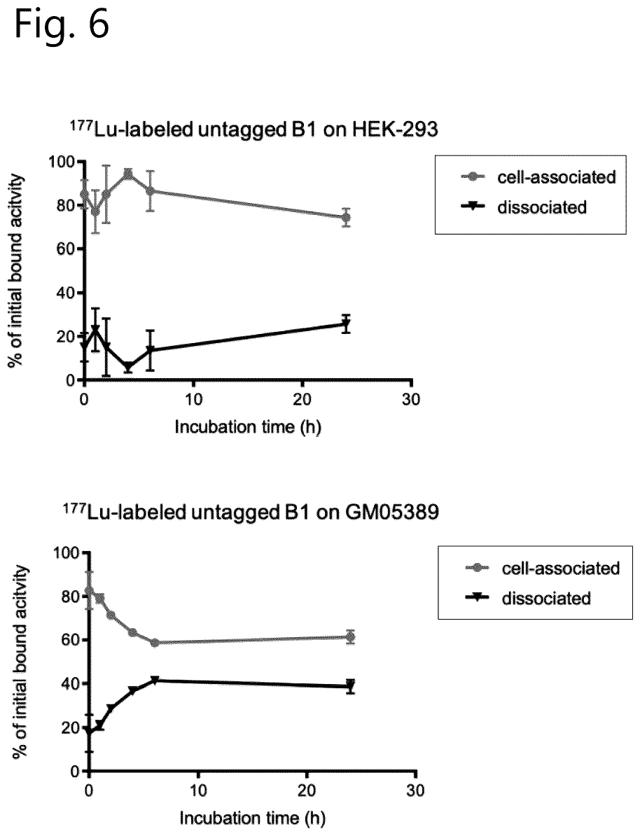

The terms ‘competing (with)’, ‘cross-blocking’, ‘cross-binding’ and ‘cross-inhibiting’ as used interchangeably herein, generally refer to an antibody fragment such as a VHH, as disclosed herein that can interfere with the binding of other antibody or other single-domain antibody fragment or other molecule as disclosed herein to human and/or murine FAP, as measured using a suitable in vitro or in vivo assay. A preferred cell used for testing the in vitro binding to human and/or murine FAP is a cell expressing human and/or murine FAP. A preferred cell line may be GM05389, U-87 MG or transfected HEK293 as defined earlier herein. Some cells have been used in the experimental part (see example 4c). Thus, more particularly, ‘competing (with)’, ‘cross-blocking’, ‘cross-binding’ and ‘cross-inhibiting’ using an antibody fragment as disclosed herein may mean interfering with or competing with the binding of another antibody or single-domain antibody fragment as disclosed herein with human and/or murine FAP, thereby reducing that binding by at least 10% but preferably at least 20%, for example by at least 50%, at least 60%, at least 70%, at least 80%, at least 90%, at least 95% or more, as measured using a suitable in vitro, cellular or in vivo assay, compared to the binding of that other single-domain antibody fragment as disclosed herein with human and/or murine FAP but without using the ‘cross-blocking’ single-domain antibody fragment as disclosed herein. In an embodiment, the antibody fragment of the invention does not compete with the ligand of FAP for binding to it. As a result, the antibody fragment of the invention is also expected not to interfere with the natural function of this receptor. It means that in an embodiment, the antibody fragment of the invention does not compete with the natural ligand of human and/or murine FAP and therefore is not inhibited to bind to human and/or murine FAP-expressing cells in vitro or in an in vivo or ex vivo setting. All antibody fragments specifically exemplified in the experimental part do not substantially compete with the natural ligand of human and/or murine FAP since a substantial FAP activity is still detectable when the antibody fragment of the invention is bound to it (see example 4d). A FAP activity is preferably in this context FAP dipeptidyl peptidase activity or a gelatinase activity. Within the context of the invention, ‘substantial’ may mean at least 40%, at least 50%, at least 60%, at least 70%, at least 80%, at least 90%, at least 95% or 100% of a FAP activity is still detectable compared to the same FAP activity when the antibody fragment is not present. Accordingly, in an embodiment, an antibody fragment of the invention does not substantially alter a FAP activity, it means it preferably does not substantially inhibit a FAP activity. Within the context of the invention, a FAP activity may be an exopeptidase and/or an endopeptidase activity. This exopeptidase activity may be a FAP dipeptidyl peptidase activity. In an embodiment, an antibody fragment does not substantially inhibit the FAP dipeptidyl peptidase activity. This endopeptidase activity may be a gelatinase and/or collagenase activity. In an embodiment, an antibody fragment does not substantially inhibit the FAP gelatinase and/or collagenase activity of FAP. Therefore, in an embodiment, the antibody fragment is not a modulator of human and/or murine FAP. In an embodiment, it is not an inhibitor and it is not an activator of human and/or murine FAP. Any of the FAP activities may be assessed as illustrated in Juillerat-Jeanneret L. et al, (2017), Expert Opinion on Therapeutic Targets (http://dx.doi.org/10.1080/14728222.2017.1370455). The FAP dipeptidyl peptidase activity may be assessed using techniques known to the skilled person such as the one used in example 4d. In short, the human FAP enzymatic activity may be measured using the fluorogenic substrate benzyloxycarbonyl-Gly-Pro-7-amido-4-methylcoumarin (Z-Gly-Pro-AMC; Bachem). Human FAP recombinant protein (Example 4a) may be diluted to 200 ng/ml in assay buffer (50 mM Tris-HCl, 1 M NaCl, 0.1% BSA, pH 7.5) in a black 96-well flat bottom plate, in absence or presence of 1 μM HA-His₆-tagged VHH. As an inhibitor control, 1 μM Talabostat mesylate (ApexBio) was added instead of the antibody fragment. After 1 h incubation to reach binding equilibrium, Z-Gly-Pro-AMC substrate was added at a final concentration of 50 μM. Enzymatic conversion of the substrate into Z-Gly-Pro and 7-amino-4-methylcoumarin (AMC) was followed using a fluorescence microplate reader (BioTek) with excitation at 380 nm and emission detection at 460 nm. Fluorescence was measured every minute during 1 h. The slope of the curves corresponds to the rate of enzymatic activity (see FIG. 4 as an example). An inhibitor control may also be used. A suitable inhibitor is Talabostat metansine (ApexBio).

An antibody fragment, such as a VHH or functional fragments thereof, as disclosed herein is said to show ‘cross-reactivity’ for two different target proteins of interest if it is specific for (as defined herein) both of these different target proteins of interest. In an embodiment, the two different target proteins of interest may be human and murine FAP.

Below we describe several structural features (i.e. first, second, third, fourth structural features) of the antibody fragment of the invention. The antibody fragment of the invention may be characterized by the presence of at least one, or all of these four structural features:
third and fourth structural features,
first and second structural features,
first, second and third structural features
third and fourth structural features,
third structural feature,
fourth structural feature,
First, third and fourth structural features,
Second, third and fourth structural features,
First and third structural features,
First and fourth structural features,
Second and third structural features,
Second and fourth structural features
First, second, third and fourth structural features.

First Structural Feature of the Antibody Fragment: Based on the Contacted Region of FAP

A first structural feature is that the antibody fragment of the invention contacts or binds or specifically binds or interacts to a region of human FAP comprised within amino acid 26 to 760 of SEQ ID NO:26. The region within amino acid 26 to 760 of SEQ ID NO:26 specifically bound or targeted by the antibody fragment of the invention may be a linear region (i.e. linear epitope or sequential epitope) within said primary amino acid sequence. Alternatively said region may not be linear and may correspond to a conformational epitope. Usually a linear epitope comprises a linear sequence of amino acids that has a length of 5 to 30 amino acids, that is to say that it may have a length of 5, 6, 7, 8, 9,10, 11, 12, 13, 14, 15, 16, 17, 18, 19, 20, 21, 22, 23, 24, 25, 26, 27, 28, 29 or 30 amino acids. Usually a conformational epitope is characterized by a number of non-consecutive amino acids within amino acid 26 to 760 of SEQ ID NO:26 that come together in the three-dimensional tertiary structure of the protein and that are contacted by the antibody fragment.

In the following paragraph dedicated to the second structural feature of the antibody fragment, linear epitopes and conformational epitope of the antibody fragment are defined.

Second Structural Feature of the Antibody Fragment: Based on the Epitope of the Antibody Fragment

The antibody fragment of the invention that specifically binds an epitope of human and/or murine FAP may alternatively or in combination with the first structural feature defined above also be further defined by a second structural feature defined below.

A second structural feature is that the antibody fragment of the invention contacts or binds or specifically binds to a number of amino acids within amino acid 26 to 760 of SEQ ID NO:26. These specific amino acids within amino acid 26 to 760 of SEQ ID NO:26 are further defined below.

In a first embodiment of this second structural feature, the antibody fragment of the invention contacts or binds or specifically binds to at least one of amino acid comprised within 65-90 and/or 101-140 of SEQ ID NO:26. Each combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 or 66 amino acids from the 66 amino acids identified is therefore encompassed to be contacted by the antibody fragment of the invention.

In a second embodiment of this second structural feature, the antibody fragment of the invention contacts or binds or specifically binds to at least one of amino acid I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and Y458 of SEQ ID NO:26. Each combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 amino acids from the 37 amino acids identified is therefore encompassed to be contacted by the antibody fragment of the invention.

In a third embodiment of this second structural feature, the stretch of amino acids 65-90 of SEQ ID NO:1 defines a first region of hFAP, which is contacted, bound or specifically bound by the antibody fragment. Not each amino acid within this stretch or region may be contacted, bound or specifically bound by the antibody fragment. In an embodiment, 1, 2, 3, 4, 5, 6, 7, 8, 9, 10, 11, 12, 13, 14, 15, 16, 17, 18, 19 or 20 amino acids of this stretch or region is contacted, bound or specifically bound by the antibody fragment. In an embodiment, this first stretch or region is an epitope of the antibody fragment. In an embodiment, an epitope of the antibody fragment is comprised within this first stretch or region.

Preferably within this first stretch at least one of Q65, E66, I76, V77, L78, Y79, N80, I81, E82, T83, G84, Q85, S86, Y87, T88, I89, L90 is contacted, bound or specifically bound by the antibody fragment.

More preferably within this first stretch at least two of Q65, E66, I76, V77, L78, Y79, N80, I81, E82, T83, G84, Q85, S86, Y87, T88, I89, L90 is contacted, bound or specifically bound by the antibody fragment.

Even more preferably within this first stretch at least three of Q65, E66, I76, V77, L78, Y79, N80, I81, E82, T83, G84, Q85, S86, Y87, T88, I89 and L90 are contacted, bound or specifically bound by the antibody fragment.

Even more preferably, preferably, within this first stretch all Q65, E66, I76, V77, L78, Y79, N80, I81, E82, T83, G84, Q85, S86, Y87, T88, I89, L90 are contacted, bound or specifically bound by the antibody fragment.

Most preferably, within this first stretch all Q65, E66, I76, V77, L78, N80, I81, E82, T83, Q85, S86, Y87, T88, I89, L90 are contacted, bound or specifically bound by the antibody fragment.

In a fourth embodiment of this second structural feature, the stretch of amino acids 101-140 of SEQ ID NO:26 defines a second region of hFAP, which is contacted, bound or specifically bound by the antibody fragment. Not each amino acid within this stretch or region needs to be contacted, bound or specifically bound by the antibody fragment. In an embodiment, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids of this stretch or region is contacted, bound or specifically bound by the antibody fragment. In an embodiment, this second stretch or region is an epitope of the antibody fragment. In an embodiment, an epitope of the antibody fragment is comprised within this second stretch or region.

Preferably within this second stretch at least one of L105, S106, P107, D108, R109, Q110, F111, D134, L135, S136, N137 is contacted, bound or specifically bound by the antibody fragment. More preferably within this second stretch at least two of L105, S106, P107, D108, R109, Q110, F111, D134, L135, S136, N137 is contacted, bound or specifically bound by the antibody fragment.

Even more preferably within this second stretch at least three of L105, S106, P107, D108, R109, Q110, F111, D134, L135, S136, N137 is contacted, bound or specifically bound by the antibody fragment.

Most preferably within this second stretch all L105, S106, P107, D108, R109, Q110, F111, D134, L135, S136, N137 are contacted, bound or specifically bound by the antibody fragment.

In a fifth embodiment of this second structural feature, the antibody fragment further contacts additional amino acids as I62, S63, G64, S91, V158, G159, R175, D457 and/or Y458 of SEQ ID NO:26.

In a sixth embodiment of this second structural feature, each of the first and second stretches or regions defined above is contacted, bound or specifically bound by the antibody fragment. In an embodiment, the combination of these two stretches defines the conformational epitope of the antibody fragment. In an embodiment, a conformational epitope is comprised within the combination of these two stretches. Not each amino acid within each of these stretches or regions may be contacted, bound or specifically bound by the antibody fragment. In an embodiment, 1, 2, 3, 4, 5 or 6 amino acids (or more depending on the length of each stretch) of each of the stretches or regions is contacted, bound or specifically bound by the antibody fragment. In an embodiment, the antibody fragment further contacts additional amino acids as I62, S63, G64, S91, V158, G159, R175, D457 and/or Y458 of SEQ ID NO:26.

In an embodiment, there is provided an antibody fragment that specifically binds an epitope of human FAP wherein the epitope is comprised within the amino acid stretch or region 65-90 and/or 101-140 of SEQ ID NO:26. Optionally, the antibody fragment further contacts additional amino acids as I62, S63, G64, S91, V158, G159, R175, D457 and/or Y458 of SEQ ID NO:26.

In an embodiment, there is provided an antibody fragment that specifically binds an epitope of human FAP wherein the epitope is comprised within the combination of amino acid stretches or regions 65-90 and 101-140 of SEQ ID NO:26. Optionally, the antibody fragment further contacts additional amino acids as I62, S63, G64, S91, V158, G159, R175, D457 and/or Y458 of SEQ ID NO:26.

In an embodiment, the antibody fragment of the invention contacts or binds or specifically binds to the following amino acids of SEQ ID NO:26:
1) at least one, or at least two or at least three amino acids selected from: I62, S63, G64, Q65, E66, and/or
2) at least one, or at least two or at least three amino acids selected from: I76, V77, L78, Y79, N80, I81, E82, T83, G84, Q85, S86, Y87, T88, I89, L90, S91, and/or
3) at least one, or at least two or at least three amino acids selected from L105, S106, P107, D108, R109, Q110, F111, and/or
4) at least one, or at least two or at least three amino acids selected from D134, L135, S136, N137, and/or
5) V158 and/or G159 and/or
6) R175, and/or
7) D457 and/or Y458.

In an embodiment, the antibody fragment contacts or binds or specifically binds to the following amino acids of SEQ ID NO:26:
1) at least one, or at least two or at least three amino acids selected from: I62, S63, G64, Q65, E66, and
2) at least one, or at least two or at least three amino acids selected from: I76, V77, L78, Y79, N80, I81, E82, T83, G84, Q85, S86, Y87, T88, I89, L90, S91, and
3) at least one, or at least two or at least three amino acids selected from L105, S106, P107, D108, R109, Q110, F111, and
4) at least one, or at least two or at least three amino acids selected from D134, L135, S136, N137, and
5) V158 and/or G159 and
6) R175, and
7) D457 and/or Y458.

In an embodiment, the antibody fragment contacts or binds or specifically binds to the following amino acids of SEQ ID NO:26:
1) at least two or at least three amino acids selected from: I62, S63, G64, Q65, E66, and/or
2) at least two or at least three amino acids selected from: I76, V77, L78, Y79, N80, I81, E82, T83, G84, Q85, S86, Y87, T88, I89, L90, S91, and/or
3) at least two or at least three amino acids selected from L105, S106, P107, D108, R109, Q110, F111, and/or
4) at least two or at least three amino acids selected from D134, L135, S136, N137, and/or 5) V158 and G159 and/or
6) R175, and/or
7) D457 and Y458.

In an embodiment, the antibody fragment contacts or binds or specifically binds to the following amino acids of SEQ ID NO:26:
1) at least two or at least three amino acids selected from: I62, S63, G64, Q65, E66, and
2) at least two or at least three amino acids selected from: I76, V77, L78, Y79, N80, I81, E82, T83, G84, Q85, S86, Y87, T88, I89, L90, S91, and
3) at least two or at least three amino acids selected from L105, S106, P107, D108, R109, Q110, F111, and
4) at least two or at least three amino acids selected from D134, L135, S136, N137, and
5) V158 and G159 and
6) R175, and
7) D457 and Y458.

In an embodiment, the antibody fragment contacts or binds or specifically binds to the following amino acids of SEQ ID NO:26:
1) at least three amino acids selected from: I62, S63, G64, Q65, E66, and/or
2) at least three amino acids selected from: I76, V77, L78, Y79, N80, I81, E82, T83, G84, Q85, S86, Y87, T88, I89, L90, S91, and/or
3) at least three amino acids selected from L105, S106, P107, D108, R109, Q110, F111, and/or
4) at least three amino acids selected from D134, L135, S136, N137, and/or
5) V158 and G159 and/or
6) R175, and/or
7) D457 and Y458.

In an embodiment, the antibody fragment contacts or binds or specifically binds to the following amino acids of SEQ ID NO:26:
1) at least three amino acids selected from: I62, S63, G64, Q65, E66, and
2) at least three amino acids selected from: I76, V77, L78, Y79, N80, I81, E82, T83, G84, Q85, S86, Y87, T88, I89, L90, S91, and
3) at least three amino acids selected from L105, S106, P107, D108, R109, Q110, F111, and 4) at least three amino acids selected from D134, L135, S136, N137, and
5) V158 and G159 and
6) R175, and
7) D457 and Y458.

In an embodiment, the following amino acids of SEQ ID NO:26 are bound or contacted or interacted with the antibody fragment: I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and Y458.

In an embodiment, the following amino acids of SEQ ID NO:26 are bound or contacted or interacted with the antibody fragment: I62, S63, G64, Q65, E66, I76, V77, L78, N80, I81, E82, T83, Q85, S86, Y87, T88, I89, L90, S91, L105, S106, P107, D108, R109, Q110, F111, D134, L135, S136, N137, V158, G159, R175, D457 and Y458.

Third Structural Feature of the Antibody Fragment: Based on the Full Length Sequence

A third structural feature is that the antibody fragment of the invention relates to the full length amino acid sequence representing a way of defining the family of antibody fragment of the invention. The present invention discloses a family of structurally closely related antibody fragments represented by an amino acid sequence comprising, consisting of or essentially consisting of SEQ ID NO:4 or a portion thereof. Antibody fragment B1 is represented by SEQ ID NO:4 (see table 2 below).

In an embodiment, the antibody fragment of the invention may be defined by its first structural feature as defined above and its third structural feature further defined below.

In an embodiment, the antibody fragment of the invention may be defined by its second structural feature as defined above and its third structural feature further defined below.

In a first embodiment of this third structural feature, the antibody fragment of the invention is represented by an amino acid sequence that comprises or essentially consists of an amino acid sequence having at least 80% sequence identity with SEQ ID NO:4 or a portion thereof. In an embodiment, the sequence identity with this sequence is at least 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment of this third structural feature, the antibody fragment of the invention is represented by an amino acid sequence that comprises or essentially consists of an amino acid sequence having at least 81% sequence similarity with SEQ ID NO:4 or a portion thereof. In an embodiment, the sequence similarity with this sequence is at least 81%, 82%, 83%, 84%, 85%, 86%, 87%, 88%, 89%, 90%, 91%, 92%, 93%, 94%, 95%, 96%, 97%, 98%, 99% or 100%.

In a second embodiment of this third structural feature, the antibody fragment of the invention is represented by an amino acid sequence that comprises or essentially consists of an amino acid sequence having at least 80% sequence identity with SEQ ID NO:4 or a portion thereof and has a length which is ranged from the exact length of SEQ ID NO: 4 or 1, 2, 3, 4, 5, 6, 7, 8, 9, 10, 15, 20, 25, 30, 35, 40, 45, 50, 55, 60 amino acids longer than the exact length of SEQ ID NO:4.

In an embodiment of this third structural feature, the antibody fragment of the invention is represented by an amino acid sequence that comprises or essentially consists of an amino acid sequence having at least 81% sequence similarity with SEQ ID NO:4 or a portion thereof and has a length which is ranged from the exact length of SEQ ID NO: 4 or 1, 2, 3, 4, 5, 6, 7, 8, 9, 10, 15, 20, 25, 30, 35, 40, 45, 50, 55, 60 amino acids longer than the exact length of SEQ ID NO:4.

For example a tag such as a His-tag may be added to the antibody fragment of the invention. Usually His-tag comprises 4, 5, 6, 7, 8, 9, 10 Histidines. Alternative tag may be a Hemagglutinin tag (HA-tag): YPYDVPDYA (SEQ ID NO: 53); YPYDVPDYGS (SEQ ID NO: 54) or a cysteine tag (Cys tag). A cysteine tag is a tag that comprises one or several cysteines. Non-limiting examples of cysteine tags are C; GGC; SPSTPPTPSPSTPPC (SEQ ID NO: 55)

The way identity and similarity are assessed is explained in detail in the part dedicated to definition at the end of the description. Usually when identity is defined by reference to a SEQ ID NO, said identity is assessed over the whole SEQ ID NO. However, it is also encompassed by the invention that identity (or similarity) is assessed over a portion (or a fragment) of said sequence. Within this context, a portion may mean at least 50%, 60%, 70%, 80%, 90%, 95% of the length of the SEQ ID NO. The length of the sequence encompassed may still be longer than the length of the SEQ ID NO used to assess the identity (or similarity) (i.e. length being at least 50% of the length of the SEQ ID NO, 60%, 70%, 80%, 90%, the same as the one of the SEQ ID NO even though the identity (or similarity) is assessed over a portion of this SEQ ID NO, or the length being 1, 5, 10, 15, 20, 25, 30, 35, 40, 45, 50, 55, 60 amino acids longer than the exact length of SEQ ID NO.

In a third embodiment of this third structural feature, the length of the antibody fragment is from 110 to 130 amino acids or 110, 111, 112, 113, 114, 115, 116, 117, 118, 119, 120, 121, 122, 123, 124, 125, 126, 127, 128, 129 or 130 amino acids. This length does not include the length of a tag, such as a His tag that may be added to the sequence of the antibody fragment.

In an embodiment, an antibody fragment has a length which is ranged from the exact length of SEQ ID NO: 4 or 1, 2, 3, 4, 5, 6, 7, 8, 9, 10, 15, 20, 25, 30, 35, 40, 45, 50, 55, 60 amino acids longer than the exact length of SEQ ID NO:4.

In an embodiment, an antibody fragment has a length which is ranged from 110 to 130 amino acids or 110,111,112,113,114,115,116,117,118,119,120,121,122,123,124, 125,126,127,128, 129 or 130 amino acids and comprises SEQ ID NO:1, SEQ ID NO:2 and SEQ ID NO:3.

In a fourth embodiment of this third structural feature, the antibody fragment of the invention is represented by an amino acid sequence that comprises or essentially consists of an amino acid sequence having at least 80% sequence identity with at least one of SEQ ID NO: 4 or a portion thereof and the length of the antibody fragment is from 80 to 150 amino acids or 90 to 140 or 100 to 130 or 105,106,107,108,109,110,111,112,113,114,115,116,117,118,119,120,121,122,123,124, 125, 126, 127, 128, 129 or 130 amino acids. In an embodiment, the sequence identity with at least of one of these sequences is at least 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment of this third structural feature, the antibody fragment of the invention is represented by an amino acid sequence that comprises or essentially consists of an amino acid sequence having at least 81% sequence similarity with at least one of SEQ ID NO: 4 or a portion thereof and the length of the antibody fragment is from 80 to 150 amino acids or 90 to 140 or 100 to 130 or 105, 106, 107, 108,109,110,111,112,113,114,115,116,117,118,119,120,121,122, 123,124,125,126,127, 128, 129 or 130 amino acids. In an embodiment, the sequence similarity with at least of one of these sequences is at least 81%, 82%, 83%, 84%, 85%, 86%, 87%, 88%, 89%, 90%, 91%, 92%, 93%, 94%, 95%, 96%, 97%, 98%, 99% or 100%.

In an embodiment, the antibody fragments of the invention contact, bind or specifically bind at least one of (preferably both) the stretches or regions of amino acids of SEQ ID NO:26 as defined earlier herein (i.e. amino acid stretch or region 65-90 and/or 101-140 of SEQ ID NO:26). In an embodiment, an epitope of said antibody fragment is comprised within these stretches or regions of amino acids of SEQ ID NO:26.

Moreover, in an embodiment, the antibody fragments of the invention have for conformational epitope the combination of stretches or regions of amino acids of SEQ ID NO:26 as defined earlier herein (i.e. amino acid stretches or regions 65-90 and/or 101-140 of SEQ ID NO:26). In an embodiment, a conformational epitope of said antibody fragment is comprised within these stretches or regions of amino acids of SEQ ID NO:26.

These epitopes define a family of antibody fragments. This family of antibody fragments shares at least one of these epitopes, linear epitopes and/or this conformational epitope.

In an embodiment, the antibody fragment of the invention:
is represented by an amino acid sequence that comprises or essentially consists of an amino acid sequence having at least 80% sequence identity (or similarity) with SEQ ID NO:4 or a portion thereof (third structural feature) and
which has for epitope the amino acid stretch or region comprised within 65-90 and/or 101-140 of SEQ ID NO:26 (second structural feature).

In an embodiment, the sequence identity (or similarity) with this sequence is at least 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, an antibody fragment has a length which is ranged from the exact length of SEQ ID NO: 4 or 1, 2, 3, 4, 5, 6, 7, 8, 9, 10, 15, 20, 25, 30, 35, 40, 45, 50, 55, 60 amino acids longer than the exact length of SEQ ID NO:4.

In an embodiment, the antibody fragment of the invention:
is represented by an amino acid sequence that comprises or essentially consists of an amino acid sequence having at least 80% sequence identity (or similarity) with SEQ ID NO:4 or a portion thereof (third structural feature) and
contacts or binds or specifically binds to at least one of amino acid I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and/or Y458 of SEQ ID NO:26. (second structural feature).

In an embodiment, the sequence identity (or similarity) with this sequence is at least 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, an antibody fragment has a length which is ranged from the exact length of SEQ ID NO: 4 or 1, 2, 3, 4, 5, 6, 7, 8, 9, 10, 15, 20, 25, 30, 35, 40, 45, 50, 55, 60 amino acids longer than the exact length of SEQ ID NO:4.

Each of the other embodiments of the second structural feature may be combined with each embodiment of the third structural feature.

Fourth Structural Feature: CDR/CDR Grafting

In a fourth structural feature, the antibody fragment of the invention, preferably the VHH's as disclosed herein is represented by an amino acid sequence that comprises at least one combination of CDR sequences chosen from the group comprising:
a CDR1 region comprising or consisting of or essentially consisting of SEQ ID NO: 1, a CDR2 region comprising or consisting of or essentially consisting of SEQ ID NO: 2, and a CDR3 region comprising or consisting of or essentially consisting of SEQ ID NO: 3. One or two amino acids of said CDR1, CDR2 and/or CDR3 may have been substituted by another one amino acid without substantially altering the activity of the obtained antibody fragment. The same holds for any of the frame work regions of the antibody fragment. Each of these antibody fragment variants are also encompassed within the invention.

An activity of said antibody fragment variant is a specific binding activity as earlier defined herein. Within the context of the invention, ‘substantial’ may mean at least 40%, at least 50%, at least 60%, at least 70%, at least 80%, at least 90%, at least 95% or 100% of said binding activity is still detectable compared to the activity of the antibody fragment with the initial CDR and/or FR regions.

Thus, in particular embodiments, the present invention provides heavy chain variable domains comprising the heavy chain antibodies with the (general) structure or which is derived therefrom:
FR1-CDR1-FR2-CDR2-FR3-CDR3-FR4
   in which FR1 to FR4 refer to framework regions 1 to 4, respectively, and in which CDR1 to CDR3 refer to the complementarity determining regions 1 to 3, respectively, and are as further defined herein (see table 2 listing the amino acid sequences of heavy chain variable domains that have been raised against human and/or murine FAP).

TABLE 2
CDR and FR of antibody fragments B1 (using the IMGT nomenclature as defined
later on herein in the general part of the description dedicated to the
general definition of the invention)

| | Amino acid sequence |
|---|---|
| B1 (SEQ ID NO: 4) | DVQLVESGGGLVQPGGSLRLSCVASGRLSSSNSMAWYRQVPGKRREL |
| | VAGITGGGETNYADFVGGRFTISRDNAKNGLYLQLNGLKPEDTAAYYCN |
| | FWPPLINYWGQGTQVTVSS |
| CDR of B1 | |
| CDR1 (SEQ ID NO: 1) | GRLSSSNS |
| CDR2 (SEQ ID NO: 2) | ITGGGET |
| CDR3 (SEQ ID NO: 3) | NFWPPLINY |
| FR of B1 | |
| FR1 (SEQ ID NO: 37) | DVQLVESGGGLVQPGGSLRLSCVAS |
| FR2 (SEQ ID NO: 38) | MAWYRQVPGKRRELVAG |
| FR3 (SEQ ID NO: 39) | NYADFVGGRFTISRDNAKNGLYLQLNGLKPEDTAAYYC |
| FR4 (SEQ ID NO: 40) | WGQGTQVTVSS |

It should be noted that the invention is not limited as to the origin of the antibody fragment, preferably VHH or fragments thereof disclosed herein (or of the nucleotide sequences to express these), nor as to the way that the antibody fragment, preferably VHH or fragments thereof or nucleotide sequences disclosed herein are (or have been) generated or obtained. Thus, the antibody fragment, preferably VHH or fragment thereof disclosed herein may be naturally occurring amino acid sequences (from any suitable species) or synthetic or semi-synthetic amino acid sequences. Methods for isolating antibody fragment and methods of producing antibody fragment as well as nucleic acid molecule encoding the antibody fragment, constructs comprising these nucleic acid molecules and cells comprising these constructs are disclosed in detail in the definition part at the end of the description.

In a specific but non-limiting aspect of the invention, the amino acid sequence of the antibody fragment is a naturally occurring immunoglobulin sequence (from any suitable species) or a synthetic or semi-synthetic immunoglobulin sequence, including but not limited to “humanized” immunoglobulin sequences (such as partially or fully humanized mouse or rabbit immunoglobulin sequences, and in particular partially or fully humanized VHH sequences), “camelized” immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing. Also, an antibody fragment, preferably a VHH or fragments thereof as disclosed herein may be suitably humanized, as further described herein, so as to provide one or more further (partially or fully) humanized amino acid sequences of the invention.

In an embodiment, the antibody fragment of the invention is derived from the antibody fragments described above using CDR grafting.

Preferred antibody fragments comprise a:
CDR1 region comprising or consisting of or essentially consisting of SEQ ID NO: 1, a CDR2 region comprising or consisting of or essentially consisting of SEQ ID NO: 2, and a CDR3 region comprising or consisting of or essentially consisting of SEQ ID NO: 3. FR regions may be as identified in table 2. Alternatively, FR regions may be from another antibody fragment.
CDR1 region comprising or consisting of or essentially consisting of SEQ ID NO: 1 and a CDR3 region comprising or consisting of or essentially consisting of SEQ ID NO: 3, FR regions may be as identified in table 2. Alternatively, FR regions may be from another antibody fragment. CDR2 region is from another antibody fragment.
CDR2 region comprising or consisting of or essentially consisting of SEQ ID NO: 2 and a CDR3 region comprising or consisting of or essentially consisting of SEQ ID NO: 3, FR regions may be as identified in table 2. Alternatively, FR regions may be from another antibody fragment. CDR1 region is from another antibody fragment.
CDR1 region comprising or consisting of or essentially consisting of SEQ ID NO: 1 and a CDR2 region comprising or consisting of or essentially consisting of SEQ ID NO: 2, FR regions may be as identified in table 2. Alternatively, FR regions may be from another antibody fragment. CDR3 region is from another antibody fragment.
CDR1 region comprising or consisting of or essentially consisting of SEQ ID NO: 1 and it comprises a CDR2 and CDR3 regions from another antibody fragment and may have the FR of as identified in table 2. Alternatively, FR regions may be from another antibody fragment.
CDR2 region comprising or consisting of or essentially consisting of SEQ ID NO: 2 and it comprises a CDR1 and CDR3 regions from another antibody fragment and may have the FR as identified in table 2. Alternatively, FR regions may be from another antibody fragment.
CDR3 region comprising or consisting of or essentially consisting of SEQ ID NO: 3 and it comprises a CDR1 and CDR2 regions from another antibody fragment and may have the FR as identified in table 2. Alternatively, FR regions may be from another antibody fragment.

Similarly, when an amino acid sequence comprises a synthetic or semi-synthetic sequence (such as a partially humanized sequence), said sequence may optionally be further suitably humanized, again as described herein, so as to provide one or more further (partially or fully) humanized amino acid sequences as disclosed herein. At the end of the description, a more detailed definition of “agonist” “antagonist”, “variants of antibody fragment”, “posttranslational structural characterization of antibody fragment” is provided.

In particular, humanized antibody fragment, preferably VHH may be represented by amino acid sequences in which at least one amino acid residue is present (and in particular, in at least one of the framework residues) that is and/or that corresponds to a humanizing substitution. In addition, or alternatively, other potentially useful humanizing substitutions can be ascertained by comparing the sequence of the framework regions of a naturally occurring VHH sequence with the corresponding framework sequence of one or more closely related human V_{H} sequences, after which one or more of the potentially useful humanizing substitutions (or combinations thereof) thus determined can be introduced into said VHH sequence (in any manner known per se, as further described herein) and the resulting humanized VHH sequences or functional fragments thereof can be tested for affinity for the target, for stability, for ease and level of expression, and/or for other desired properties. In this way, by means of a limited degree of trial and error, other suitable humanizing substitutions (or suitable combinations thereof) can be determined by the skilled person.

In an embodiment, the antibody fragment of the invention may be defined by its first structural feature as defined above and its fourth structural feature further defined herein.

In an embodiment, the antibody fragment of the invention may be defined by its second structural feature as defined above and its fourth structural feature further defined herein.

In an embodiment, the antibody fragment of the invention may be defined by its second structural feature as defined above, its third structural feature and its fourth structural feature further defined herein.

In an embodiment, the antibody fragments of the invention contact, bind or specifically bind at least one of (preferably both) the stretches or regions of amino acids of SEQ ID NO:26 as defined earlier herein (i.e. amino acid stretch or region 65-90 and/or 101-140 of SEQ ID NO:26). In an embodiment, an epitope of said antibody fragment is comprised within these stretches or regions of amino acids of SEQ ID NO:26.

Moreover, the antibody fragment of the invention have for conformational epitope the combination of stretches or regions of amino acids of SEQ ID NO:26 as defined earlier herein (i.e. amino acid stretches or regions 65-90 and/or 101-140 of SEQ ID NO:26). In an embodiment, a conformational epitope of said antibody fragment is comprised within the combination of these stretches or regions of amino acids of SEQ ID NO:26.

These epitopes define a family of antibody fragments. This family of antibody fragments shares at least one of these epitopes, linear epitopes and/or this conformational epitope.

Preferred antibody fragments comprise a:
CDR1 region comprising or consisting of or essentially consisting of SEQ ID NO: 1, a CDR2 region comprising or consisting of or essentially consisting of SEQ ID NO: 2, and a CDR3 region comprising or consisting of or essentially consisting of SEQ ID NO: 3. FR regions may be as identified in table 2. Alternatively, FR regions may be from another antibody fragment.
CDR1 region comprising or consisting of or essentially consisting of SEQ ID NO: 1 and a CDR3 region comprising or consisting of or essentially consisting of SEQ ID NO: 3, FR regions may be as identified in table 2. Alternatively, FR regions may be from another antibody fragment. CDR2 region is from another antibody fragment.
CDR2 region comprising or consisting of or essentially consisting of SEQ ID NO: 2 and a CDR3 region comprising or consisting of or essentially consisting of SEQ ID NO: 3, FR regions may be as identified in table 2. Alternatively, FR regions may be from another antibody fragment. CDR1 region is from another antibody fragment.
CDR1 region comprising or consisting of or essentially consisting of SEQ ID NO: 1 and a CDR2 region comprising or consisting of or essentially consisting of SEQ ID NO: 2, FR regions may be as identified in table 2. Alternatively, FR regions may be from another antibody fragment. CDR3 region is from another antibody fragment.
CDR1 region comprising or consisting of or essentially consisting of SEQ ID NO: 1 and it comprises a CDR2 and CDR3 regions from another antibody fragment and may have the FR of as identified in table 2. Alternatively, FR regions may be from another antibody fragment.
CDR2 region comprising or consisting of or essentially consisting of SEQ ID NO: 2 and it comprises a CDR1 and CDR3 regions from another antibody fragment and may have the FR as identified in table 2. Alternatively, FR regions may be from another antibody fragment.
CDR3 region comprising or consisting of or essentially consisting of SEQ ID NO: 3 and it comprises a CDR1 and CDR2 regions from another antibody fragment and may have the FR as identified in table 2. Alternatively, FR regions may be from another antibody fragment (fourth structural feature)
   and
has an epitope comprised within the amino acid stretch or region comprised within 65-90 and/or 101-140 of SEQ ID NO:26 (second structural feature).

Preferred antibody fragments comprise a:
CDR1 region comprising or consisting of or essentially consisting of SEQ ID NO: 1, a CDR2 region comprising or consisting of or essentially consisting of SEQ ID NO: 2, and a CDR3 region comprising or consisting of or essentially consisting of SEQ ID NO: 3. FR regions may be as identified in table 2. Alternatively, FR regions may be from another antibody fragment.
CDR1 region comprising or consisting of or essentially consisting of SEQ ID NO: 1 and a CDR3 region comprising or consisting of or essentially consisting of SEQ ID NO: 3, FR regions may be as identified in table 2. Alternatively, FR regions may be from another antibody fragment. CDR2 region is from another antibody fragment.
CDR2 region comprising or consisting of or essentially consisting of SEQ ID NO: 2 and a CDR3 region comprising or consisting of or essentially consisting of SEQ ID NO: 3, FR regions may be as identified in table 2. Alternatively, FR regions may be from another antibody fragment. CDR1 region is from another antibody fragment.
CDR1 region comprising or consisting of or essentially consisting of SEQ ID NO: 1 and a CDR2 region comprising or consisting of or essentially consisting of SEQ ID NO: 2, FR regions may be as identified in table 2. Alternatively, FR regions may be from another antibody fragment. CDR3 region is from another antibody fragment.
CDR1 region comprising or consisting of or essentially consisting of SEQ ID NO: 1 and it comprises a CDR2 and CDR3 regions from another antibody fragment and may have the FR of as identified in table 2. Alternatively, FR regions may be from another antibody fragment.
CDR2 region comprising or consisting of or essentially consisting of SEQ ID NO: 2 and it comprises a CDR1 and CDR3 regions from another antibody fragment and may have the FR as identified in table 2. Alternatively, FR regions may be from another antibody fragment.
CDR3 region comprising or consisting of or essentially consisting of SEQ ID NO: 3 and it comprises a CDR1 and CDR2 regions from another antibody fragment and may have the FR as identified in table 2. Alternatively, FR regions may be from another antibody fragment (fourth structural feature)
   and
contacts or binds or specifically binds to at least one of amino acid I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and/or Y458 of SEQ ID NO:26. (second structural feature).

Each of the other embodiments of the second structural feature may be combined with each embodiment of the fourth structural feature.

In an embodiment, the antibody fragment, preferably a VHH of the invention (or a fragment thereof) is represented by a first and/or second and/or third and/or fourth structural feature as identified herein. Alternatively or in combination with said structural feature, said antibody fragment, preferably a VHH of the invention (or a fragment thereof) is characterized by at least one of the following functional features:
which is to specifically bind human and/or murine FAP, preferably which is to specifically bind human and murine FAP and
which is not to modulate a FAP activity.

The specific binding has been described earlier herein. Most preferably, the antibody fragment specifically binds to human and/or murine FAP with a K_{D} ranged from 10⁻⁹ to 10⁻¹² moles/liter and/or a k_{off} ranging from 10⁻² to 10⁻⁵ s⁻¹ preferably assessed using bio-layer interferometry, more preferably with a K_{D} ranged from 10⁻⁹ to 10⁻¹² moles/liter and a k_{off} ranging from 10⁻² to 10⁻⁵ s⁻¹.

The second functional feature relating to the fact the antibody fragment may not be a modulator (i.e. is not an inhibitor, is not an activator of human and/or murine FAP) has also been described in detail herein.

In an embodiment, an antibody fragment, VHH or fragment of a VHH of the invention should therefore fulfil at least one of the structural features and/or at least one of the functional features.

Additional Antibody Fragments

In a further aspect there is provided additional antibody fragments having similar structural and/or functional characteristics as the ones disclosed before, only difference being their full length sequence, their CDR1, CDR2, CDR3, FR1, FR2, FR3, FR4 sequences defined below (see table 3 and further text).

These additional antibody fragments such as a single-domain antibody fragments, preferably VHH's or fragments thereof may be characterized by a functional feature and/or by a structural feature. Examples of structural features are sequence related and examples of functional features are related to an activity of said antibody fragment (i.e. a binding activity and the absence of modulating a FAP activity all earlier defined for antibody fragments as earlier defined herein). The wording present earlier herein and related to the definition of an “antibody fragment”, of “its activity”, of “its binding”, “cross-binding” (i.e. competing with), “affinity”, “avidity”, and/or “specificity” also applied to these additional antibody fragments. Unless otherwise indicated, all definitions relating to an antibody fragment of the first aspect also apply to an additional antibody fragment. The same holds for all subsequent aspects of the inventions wherein an antibody fragment is used: compound comprising an antibody fragment, compound for targeting applications, labelled compound, composition, kit, diagnostic use of the antibody fragment and of the labelled compound, therapeutic use of the labelled compound.

In an embodiment, the additional antibody fragments of the invention contact, bind or specifically bind at least one of (preferably both) the stretches or regions of amino acids of SEQ ID NO:26 as defined earlier herein (i.e. amino acid stretch or region 65-90 and/or 101-140 of SEQ ID NO:26). In an embodiment, an epitope of said antibody fragment is comprised within these stretches or regions of amino acids of SEQ ID NO:26.

Moreover, in an embodiment, the additional antibody fragments of the invention have for conformational epitope the combination of stretches or regions of amino acids of SEQ ID NO:26 as defined earlier herein (i.e. amino acid stretches or regions 65-90 and/or 101-140 of SEQ ID NO:26).

In an embodiment, a conformational epitope of said antibody fragment is comprised within the combination of these stretches or regions of amino acids of SEQ ID NO:26.

These epitopes define a family of antibody fragments. This family of antibody fragments shares at least one of these epitopes, linear epitopes and/or this conformational epitope.

These additional antibody fragments could be used the same way as the other ones. All functional definitions provided earlier or later herein also apply to this additional antibody fragment. The first and second structural feature of the antibody fragment already defined herein also apply to these additional antibody fragments.

Below we describe several additional structural features of the additional antibody fragment of the invention. The antibody fragment of the invention may be characterized by the presence of at least one, at least two, at least three or all of these structural features: first, second, third and fourth structural features.

An antibody fragment that specifically binds human and/or murine FAP and wherein said antibody fragment is represented by an amino acid sequence that comprises an amino acid sequence having at least 85% sequence identity with at least one of SEQ ID NO:8, 5, 6, 7, 12, 9, 10, 11 or a portion thereof.

In an embodiment, the sequence identity with any of this sequence is at least 85%, 86%, 87%, 88%, 89%, 90%, 91%, 92%, 93%, 94%, 95%, 96%, 97%, 98%, 99% or 100%.

An antibody fragment that specifically binds human and/or murine FAP and wherein said antibody fragment is represented by an amino acid sequence that comprises an amino acid sequence having at least 80% sequence identity with at least one of SEQ ID NO 16, 13, 14, 15 or a portion thereof.

In an embodiment, the sequence identity with any of this sequence is at least 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, the antibody fragment of the invention is represented by an amino acid sequence that comprises or essentially consists of an amino acid sequence having at least 91% sequence similarity with SEQ ID NO: 8, 5, 6, 7 or a portion thereof. In an embodiment, the sequence similarity with this sequence is at least 91%, 92%, 93%, 94%, 95%, 96%, 97%, 98%, 99% or 100%.

In an embodiment, the antibody fragment of the invention is represented by an amino acid sequence that comprises or essentially consists of an amino acid sequence having at least 89% sequence similarity with SEQ ID NO: 9, 10, 11, 12 or a portion thereof. In an embodiment, the sequence similarity with this sequence is at least 89%, 90%, 91%, 92%, 93%, 94%, 95%, 96%, 97%, 98%, 99% or 100%.

In an embodiment, the antibody fragment of the invention is represented by an amino acid sequence that comprises or essentially consists of an amino acid sequence having at least 81% sequence similarity with SEQ ID NO: 13, 14, 15, 16 or a portion thereof. In an embodiment, the sequence similarity with this sequence is at least 81%, 82%, 83%, 84%, 85%, 86%, 87%, 88%, 89%, 90%, 91%, 92%, 93%, 94%, 95%, 96%, 97%, 98%, 99% or 100%.

In an embodiment, the length of the additional antibody fragment is from 110 to 130 amino acids or 110,111,112,113,114,115,116,117,118,119,120,121,122,123,124, 125,126,127,128, 129 or 130 amino acids. This length does not include the length of a tag, such as a His tag that may be added to the sequence of the antibody fragment. Some His tags have been already defined earlier herein.

In an embodiment, the additional antibody fragment of the invention:
comprises an amino acid sequence having at least 85% sequence identity with at least one of SEQ ID NO:8, 5, 6, 7, 12, 9, 10, 11 or a portion thereof and
has an epitope comprised within the amino acid stretch or region comprised within 65-90 and/or 101-140 of SEQ ID NO:26 (second structural feature).

In an embodiment, the sequence identity (or similarity) with this sequence is at least 85%, 86%, 87%, 88%, 89%, 90%, 91%, 92%, 93%, 94%, 95%, 96%, 97%, 98%, 99% or 100%.

In an embodiment, the additional antibody fragment of the invention:
comprises an amino acid sequence having at least 85% sequence identity with at least one of SEQ ID NO:8, 5, 6, 7, 12, 9, 10, 11 or a portion thereof and
has a conformational epitope comprised within the combination of amino acid stretch or region comprised within 65-90 and 101-140 of SEQ ID NO:26 (second structural feature).

In an embodiment, the sequence identity (or similarity) with this sequence is at least 85%, 86%, 87%, 88%, 89%, 90%, 91%, 92%, 93%, 94%, 95%, 96%, 97%, 98%, 99% or 100%.

In an embodiment, the additional antibody fragment of the invention:
comprises an amino acid sequence having at least 85% sequence identity with at least one of SEQ ID NO:8, 5, 6, 7, 12, 9, 10, 11 or a portion thereof and
contacts or binds or specifically binds to at least one of amino acid I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and/or Y458 of SEQ ID NO:26. (second structural feature).

In an embodiment, the sequence identity (or similarity) with this sequence is at least 85%, 86%, 87%, 88%, 89%, 90%, 91%, 92%, 93%, 94%, 95%, 96%, 97%, 98%, 99% or 100%.

In an embodiment, the additional antibody fragment of the invention is derived from the antibody fragments described above using CDR grafting and using the CDR sequences of table 3, optionally combined with the CDR of table 2. The combination with the FR regions of tables 2 and 3 may also be combined.

In an embodiment, the additional antibody fragment of the invention, preferably the VHH's as disclosed herein is represented by an amino acid sequence that comprises at least one combination of CDR sequences chosen from the group comprising:
a CDR1 region comprising or consisting of or essentially consisting of SEQ ID NO: 5, 9, 13, a CDR2 region comprising or consisting of or essentially consisting of SEQ ID NO: 6, 10, 14, and a CDR3 region comprising or consisting of or essentially consisting of SEQ ID NO: 7, 11, 15. One or two amino acids of said CDR1, CDR2 and/or CDR3 may have been substituted by another one amino acid without substantially altering the activity of the obtained antibody fragment. The same holds for any of the frame work regions of the additional antibody fragment (see table 3 for the SEQ ID NO: of each of the FR regions of the additional antibody fragments concerned). Each of these antibody fragment variants are also encompassed within the invention. An activity of said antibody fragment variant is a specific binding activity as earlier defined herein. Within the context of the invention, ‘substantial’ may mean at least 40%, at least 50%, at least 60%, at least 70%, at least 80%, at least 90%, at least 95% or 100% of said binding activity is still detectable compared to the activity of the antibody fragment with the initial CDR and/or FR regions.

It is also encompassed to combine a CDR and/or a FR region of the antibody fragment of the first aspect (see table 2) with a CDR and/or FR region of an additional antibody fragment (see table 3) In an embodiment, the additional antibody fragment of the invention is defined by reference to its CDR as identified above and it has an epitope comprised within the amino acid stretch or region comprised within 65-90 and/or 101-140 of SEQ ID NO:26 (second structural feature).

In an embodiment, the additional antibody fragment of the invention is defined by reference to its CDR as identified above and it has a conformation epitope comprised within the combination of amino acid stretch or region comprised within 65-90 and 101-140 of SEQ ID NO:26 (second structural feature).

In an embodiment, the additional antibody fragment of the invention is defined by reference to its CDR as identified above and it contacts or binds or specifically binds to at least one of amino acid I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and/or Y458 of SEQ ID NO:26. (second structural feature).

TABLE 3
CDR and FR of antibody fragments B2, B3 and B4 (using the IMGT nomenclature
as defined later on herein in the general part of the description dedicated
to the general definition of the invention)

| | Amino acid sequence |
|---|---|
| B2 (SEQ ID NO: 8) | DVQLVESGGGLVQAGGSLRLSCAVSGSISSANSMGW |
| | YRQAPGKQRDVVAGLTTGGRSHYADSVKGRFTISRDN |
| | AKNTVYLQMNSLKAEDTAVYYCNLWPPVQGYWGQGT |
| | QVTVSS |
| CDR of B2 | |
| CDR1 (SEQ ID NO: 5) | GSISSANS |
| CDR2 (SEQ ID NO: 6) | LTTGGRS |
| CDR3 (SEQ ID NO: 7) | NLWPPVQGY |
| FR of B2 | |
| FR1 (SEQ ID NO: 41) | DVQLVESGGGLVQAGGSLRLSCAVS |
| FR2 (SEQ ID NO: 42) | MGWYRQAPGKQRDVVAG |
| FR3 (SEQ ID NO: 43) | HYADSVKGRFTISRDNAKNTVYLQMNSLKAEDTAVYYC |
| FR4 (SEQ ID NO: 44) | WGQGTQVTVSS |
| B3 (SEQ ID NO: 12) | DVQLVESGGGLVQAGGSLRLSCAVSGRLFSTNAMGW |
| | YRQAPGKQRELVAGITGGDRSNYADSVKGRFTISRDN |
| | GKNTLYLQMNSLKPEDTAVYYCNFYPPIVGDYWGQGT |
| | QVTVSS |
| CDR of B3 | |
| CDR1 (SEQ ID NO: 9) | GRLFSTNA |
| CDR2 (SEQ ID NO: 10) | ITGGDRS |
| CDR3 (SEQ ID NO: 11) | NFYPPIVGDY |
| FR of B3 | |
| FR1 (SEQ ID NO: 45) | DVQLVESGGGLVQAGGSLRLSCAVS |
| FR2 (SEQ ID NO: 46) | MGWYRQAPGKQRELVAG |
| FR3 (SEQ ID NO: 47) | NYADSVKGRFTISRDNGKNTLYLQMNSLKPEDTAVYYC |
| FR4 (SEQ ID NO: 48) | WGQGTQVTVSS |
| B4 (SEQ ID NO: 16) | DVQLVESGGGLVQVGGSLRLSCVASGFTFSSYYMSW |
| | VRQAPGKGLEWVASIYADGDMTYYADSVRGRFTISRD |
| | NAKNTLYLQMNSLKSEDTAVYYCAKDPLPPYHVNQGT |
| | QVTVSS |
| CDR of B4 | |
| CDR1 (SEQ ID NO: 13) | GFTFSSYY |
| CDR2 (SEQ ID NO: 14) | IYADGDMT |
| CDR3 (SEQ ID NO: 15) | AKDPLPPYH |
| FR of B4 | |
| FR1 (SEQ ID NO: 49) | DVQLVESGGGLVQVGGSLRLSCVAS |
| FR2 (SEQ ID NO: 50) | MSWVRQAPGKGLEWVAS |
| FR3 (SEQ ID NO: 51) | YYADSVRGRFTISRDNAKNTLYLQMNSLKSEDTAVYYC |
| FR4 (SEQ ID NO: 52) | VNQGTQVTVSS |

Compound Comprising the Antibody Fragment

In a further aspect, there is provided an antibody fragment, preferably a VHH or a fragment thereof as defined in the previous aspects, wherein said antibody fragment, preferably said VHH or fragment thereof is linked or coupled to an entity such as a moiety. Within the context of the invention, an antibody fragment, preferably a VHH or a fragment thereof which is linked to an entity such as a moiety may be called a compound. Within the context of the application, a compound therefore comprises, essentially consists of or consists of an antibody fragment of the invention and an entity. As earlier disclosed herein, the antibody fragment, preferably a VHH of the invention (or a fragment thereof) is represented by a structural feature as identified herein (preferably a first and/or a second structural feature). Alternatively or in combination with said structural feature, said antibody fragment, preferably a VHH of the invention (or a fragment thereof) is characterized by a functional feature which is to specifically bind human and/or murine FAP, preferably which is to specifically bind human and murine FAP. In an embodiment, said antibody fragment, preferably a VHH of the invention (or a fragment thereof) is characterized by a functional feature which is that this antibody fragment is not a modulator (i.e. is not an inhibitor, is not an activator of human and/or murine FAP). An antibody fragment, VHH or fragment of a VHH of the invention should therefore fulfil at least one of the structural features and/or functional features.

The entity and the antibody fragment may be linked or coupled to each other. An entity may be a cell as explained later herein. When the entity is a cell, the expression “antibody fragment linked or coupled to an entity” means that the antibody fragment is expressed in or on said cell. Nucleic acid molecules encoding the antibody fragment of the invention are disclosed later herein.

The identity of the moiety and/or the type of link may vary depending on the type of applications envisaged for the antibody fragment or for the moiety or for the compound. A moiety may be a molecule or a label as defined herein.

Compound for Targeting Applications In an embodiment, the moiety linked to the antibody fragment (preferably a VHH or a fragment thereof) is a molecule to be delivered to a cell, a tissue, an organ expressing human and/or murine FAP. In the context of the invention, a “compound” is or comprises or essentially consists of or consists of an antibody fragment, preferably a VHH or a fragment thereof (all as defined herein), wherein said antibody fragment is linked to a moiety, preferably a molecule to be delivered to said cell, tissue, organ. Any moiety, molecule or medicament known to act on a cell, tissue, organ expressing FAP is potentially encompassed by the present invention and could be linked to the antibody fragment of the invention. The molecule may be a peptide, a small molecule or a nucleic acid. A peptide may be a cytokine. A small molecule may be a chemotherapeutic. An entity may be a cell such as a CAR-T cell, a CAR-NK cell, a BITE or a LITE.

In an embodiment, the moiety linked to the antibody fragment is an AcTakine (Activity-on-Target cytokine) or an AcTaferon (IFNα-based AcTakine), preferably an AcTakine or an AcTaferon as described in WO2017077382A1, WO2017134301A1, WO2017194783A1, WO2017194782A2, WO2018077893A1, WO2018141964A1, WO2018144999A1, WO2019032661A1, WO2019032663A1, WO2019032662A1, WO2019148089A1, WO2019191519A1 or WO2020033646A1. In this embodiment, the moiety linked to the antibody fragment is preferably a medicament for cancer. Moreover, the compound comprising the moiety linked to the antibody fragment is preferably a medicament for cancer.

In an embodiment, the moiety linked to the antibody fragment is a pyrrolobenzodiazepine; preferably a pyrrolobenzodiazepine dimer such as described in WO2014057074A1, WO2015052322A1, WO2014140174A1, WO2015052321A1, WO2017186894A1, WO2017137555A1, WO2017137553A1, WO2016038383A1 or WO2018192944A1; more preferably herein said pyrrolobenzodiazepine dimer is selected from the group consisting of:
(11S,11aS)-4-((2R,5R)-37-(2,5-dioxo-2,5-dihydro-1H-pyrrol-1-yl)-5-isopropyl-2-methyl-4,7,35-trioxo-10,13,16,19,22,25,28,31-octaoxa-3,6,34-triazaheptatriacontanamido)benzyl 11-hydroxy-8-((5-(((11 S,11 aS)-11-hydroxy-10-(((4-((10R,13R)-10-isopropyl-13-methyl-8,11-dioxo-2,5-dioxa-9,12-diazatetradecanamido)benzyl)oxy)carbonyl)-7-methoxy-2-methyl-5-oxo-5,10,11,11a-tetrahydro-1H-pyrrolo[2,1-c][1,4]benzodiazepin-8-yl)oxy)pentyl)oxy)-7-methoxy-2-methyl-5-oxo-11,11a-dihydro-1H-pyrrolo[2,1-c][1,4]benzodiazepine-10(5H)-carboxylate,
(S)-3-(2,5-dioxo-2,5-dihydro-1H-pyrrol-1-yl)-N-(2-(2-(2-(2-(4-(((8-methoxy-2-(6-methoxynaphthalen-2-yl)-5-oxo-5,11a-dihydro-1H-benzo[e]pyrrolo[1,2-a][1,4]diazepin-7-yl)oxy)methyl)-1H-1,2,3-triazol-1-yl)ethoxy)ethoxy)ethoxy)ethyl)propanamide,
(S)-3-(2,5-dioxo-2,5-dihydro-1H-pyrrol-1-yl)-N-(2-(2-(2-(2-(4-(((2-methylene-5-oxo-2,3,5,11a-tetrahydro-1H-benzo[e]pyrrolo[1,2-a][1,4]diazepin-7-yl)oxy)methyl)-1H-1,2,3-triazol-1-yl)ethoxy)ethoxy)ethoxy)ethyl)propanamide,
1-(3-(2,5-dioxo-2,5-dihydro-1H-pyrrol-1-yl)propanamido)-N-(3-(((S)-8-((5-(((S)-7-methoxy-2-methyl-5-oxo-5,11a-dihydro-1H-benzo[e]pyrrolo[1,2-a][1,4]diazepin-8-yl)oxy)pentyl)oxy)-2-methyl-5-oxo-5,11a-dihydro-1H-benzo[e]pyrrolo[1,2-a][1,4]diazepin-7-yl)oxy)propyl)-3,6,9,12,15,18,21,24-octaoxaheptacosan-27-amide,
3-(2,5-dioxo-2,5-dihydro-1H-pyrrol-1-yl)-N-(2-(2-(2-(2-(4-((((S)-8-((5-(((S)-7-methoxy-2-methyl-5-oxo-5,11a-dihydro-1H-benzo[e]pyrrolo[1,2-a][1,4]diazepin-8-yl)oxy)pentyl)oxy)-2-methyl-5-oxo-5,11a-dihydro-1H-benzo[e]pyrrolo[1,2-a][1,4]diazepin-7-yl)oxy)methyl)-1H-1,2,3-triazol-1-yl)ethoxy)ethoxy)ethoxy)ethyl)propanamide,
1-(3-(2,5-dioxo-2,5-dihydro-1H-pyrrol-1-yl)propanamido)-N—((S)-1-(((S)-1-((4-((S)-7-methoxy-8-((5-(((S)-7-methoxy-2-(4-(4-methylpiperazin-1-yl)phenyl)-5-oxo-5,11a-dihydro-1H-benzo[e]pyrrolo[1,2-a][1,4]diazepin-8-yl)oxy)pentyl)oxy)-5-oxo-5,11a-dihydro-1H-benzo[e]pyrrolo[1,2-a][1,4]diazepin-2-yl)phenyl)amino)-1-oxopropan-2-yl)amino)-3-methyl-1-oxobutan-2-yl)-3,6,9,12,15,18,21,24-octaoxaheptacosan-27-amide,
6-(2,5-dioxo-2,5-dihydro-1H-pyrrol-1-yl)-N—((S)-1-(((S)-1-((4-((S)-8-(3-(((S)-2-(3-fluoro-4-methoxyphenyl)-7-methoxy-5-oxo-5,11a-dihydro-1H-benzo[elpyrrolo[1,2-al[1,4]diazepin-8-yl)oxy)propoxy)-7-methoxy-5-oxo-5,11a-dihydro-1H-benzo[elpyrrolo[1,2-alH,41diazepin-2-yl)phenyl)amino)-1-oxopropan-2-yl)amino)-3-methyl-1-oxobutan-2-yl)hexanamide,
(R)-2-((3-Nitropyridin-2-yl)disulfanyl)propyl(11S,11aS)-11-hydroxy-7-methoxy-8-(3-(((S)-7-methoxy-2-methylene-5-oxo-2,3,5,11a-tetrahydro-1H-pyrrolo[2,1-c][1,4]benzodiazepin-8-yl)oxy)propoxy)-2-methylene-5-oxo-2,3,11,11a-tetrahydro-1 Hpyrrolo[2,1-c][1,4]benzodiazepine-10(5H)-carboxylate,
4-((2S,5S)-37-(2,5-dioxo-2,5-dihydro-1H-pyrrol-1yl)-5-isopropyl-2-methyl-4,7,35-trioxo-10,13,16,19,22,25,28,31-octaoxa-3,6,34-triazaheptatriacontanamido)benzyl(11 S,11 aS)-11-hydroxy-7-methoxy-8-(3-(((S)-7-methoxy-2-methylene-5-oxo-2,3,511a-tetrahydro-1H-benzo[e]pyrrolo[1,2-a][1,4]diazepin-8-yl)oxy)propoxy)-2-methylene-5-oxo-2,3,11,11a-tetrahydro-1H-benzo[e]pyrrolo[1,2-a][1,4]diazepine-10(5H)-carboxylate, and
4-((2S,5S)-37-(2,5-dioxo-2,5-dihydro-1H-pyrrol-1-yl)-5-isopropyl-2-methyl-4,7,35-trioxo-10,13,16,19,22,25,28,31-octaoxa-3,6,34-triazaheptatriacontanamido)benzyl(11 S,11 aS)-11-hydroxy-8-(3-(((11 S,11aS)-11-hydroxy-10-(((4-((10S,13S)-10-isopropyl-13-methyl-8,11-dioxo-2,5-dioxa-9,12-diazatetradecan-14-amido)benzyl)oxy)carbonyl)-7-methoxy-2-methylene-5-oxo-2,3,5,10,11,11a-hexahydro-1H-pyrrolo[2,1-c][1,4]benzodiazepin-8-yl)oxy)propoxy)-7-methoxy-2-methylene-5-oxo-2,3,11,11a-tetrahydro-1H-pyrrolo[2,1-c][1,4]benzodiazepine-10(5H)-carboxylate.

In the embodiment above, the moiety linked to the antibody fragment is preferably a medicament for cancer. Moreover, the compound comprising the moiety linked to the antibody fragment is preferably a medicament for cancer.

In an embodiment, the moiety linked to the antibody fragment is an octadentate thorium chelator such as described in WO2017211809A1. In this embodiment, the moiety linked to the antibody fragment is preferably a medicament for cancer. Moreover, the compound comprising the moiety linked to the antibody fragment is preferably a medicament for cancer.

In an embodiment, the moiety linked to the antibody fragment is a dolastatin or an auristatin as described in WO2015162293A1. In this embodiment, the moiety linked to the antibody fragment is preferably a medicament for cancer. Moreover, the compound comprising the moiety linked to the antibody fragment is preferably a medicament for cancer.

In an embodiment, the moiety linked to the antibody fragment is cytolysin or a Nigrin-b A-chain such as described in WO2015118030A2. In this embodiment, the moiety linked to the antibody fragment is preferably a medicament for cancer. Moreover, the compound comprising the moiety linked to the antibody fragment is preferably a medicament for cancer.

In an embodiment, the moiety linked to the antibody fragment is 2-propylthiazolo [4, 5-c] quinolin-4-amine, 1-(2-methylpropyl)-1H-imidazo[4,5-c]quinolin-4-amine,4-amino-2-(ethoxymethyl)-a,a-di-methyl-1H-imidazo[4,5-c]quinoline-1-ethanol,1-(4-amino-2-ethylaminomethylimidazo-[4,5-c]quinolin-1-yl)-2-methylpropan-2-ol,N-[4-(4-amino-2-ethyl-1H-imidazo[4,5-c]quinolin-1-yl)butyl-]methanesulfonamide,4-amino-2-ethoxymethyl-aa-dimethyl-6,7,8,9-tetrahydro-1 h-imidazo[4,5-c]quinoline-1-ethanol,4-amino-aa-dimethyl-2-methoxyethyl-1 h-imidazo[4,5-c]quinoline-1-ethanol,1-{2-[3-(benzyloxy)propoxy]ethyl}-2-(ethoxymethyl)-1H-imidazo[4,5-c]quinolin-4-amine,N-[4-(4-amino-2-butyl-1H-imidazo[4,5-c][1,5]naphthyridin-1-yl)butyl]-n′-butylurea,N1-[2-(4-amino-2-butyl-1H-imidazo[4,5-c][1,5]naphthyridin-1-yl)ethyl]-2-amino-4-methylpentanamide,N-(2-{2-[4-amino-2-(2-methoxyethyl)-1H-imidazo[4,5-c]quinolin-1-yl]ethoxy}ethyl)-n′-phenylurea,1-(2-amino-2-methylpropyl)-2-(ethoxymethyl)-1H-imidazo[4,5-c]quinolin-4-amine,1-{4-[(3,5-dichlorophenyl)sulfonyl]butyl}-2-ethyl-1H-imidazo[4,5-c]quinolin-4-amine,N-(2-{2-[4-amino-2-(ethoxymethyl)-1H-imidazo[4,5-c]quinolin-1-yl]ethoxy}ethyl)-N′-cyclohexylurea,N-{3-[4-amino-2-(ethoxymethyl)-1H-imidazo[4,5-c]quinolin-1-yl]propyl}-n′-(3-cyanophenyl)thiourea,N-[3-(4-amino-2-butyl-1H-imidazo[4,5-c]quinolin-1-yl)-2,2-dimethylpropyl]benzamide,2-butyl-1-[3-(methylsulfonyl)propyl]-1H-imidazo[4,5-c]quinolin-4-amine,N-{2-[4-amino-2-(ethoxymethyl)-1H-imidazo[4,5-c]quinolin-1-yl]-1,1-dimethylethyl}-2-ethoxyacetamide,1-[4-amino-2-ethoxymethyl-7-(pyridin-4-yl)-1H-imidazo[4,5-c]quinolin-1-yl]-2-methylpropan-2-ol,1-[4-amino-2-(ethoxymethyl)-7-(pyridin-3-yl)-1H-imidazo[4,5-c]quinolin-1-yl]-2-methylpropan-2-ol,N-{3-[4-amino-1-(2-hydroxy-2-methylpropyl)-2-(methoxyethyl)-1H-imidazo[4,5-c]quinolin-7-yl]phenyl}methanesulfonamide,1-[4-amino-7-(5-hydroxymethylpyridin-3-yl)-2-(2-methoxyethyl)-1H-imidazo[4,5-c]quinolin-1-yl]-2-methylpropan-2-ol,3-[4-amino-2-(ethoxymethyl)-7-(pyridin-3-yl)-1H-imidazo[4,5-c]quinolin-1-yl]propane-1,2-diol,1-[2-(4-amino-2-ethoxymethyl-1H-imidazo[4,5-c]quinolin-1-yl)-1,1-dimethylethyl]-3-propylurea,1-[2-(4-amino-2-ethoxymethyl-1H-imidazo[4,5-c]quinolin-1-yl)-1,1-dimethylethyl]-3-cyclopentylurea,1-[(2,2-dimethyl-11,3-dioxolan-4-yl)methyl]-2-(ethoxymethyl)-7-(4-hydroxymethylphenyl)-1H-imidazo[4,5-c]quinolin-4-amine,4-[4-amino-2-ethoxymethyl-1-(2-hydroxy-2-methylpropyl)-1H-imidazo[4,5-c]quinolin-7-yl]-N-methoxy-N-methylbenzamide,2-ethoxymethyl-N1-isopropyl-6,7,8,9-tetrahydro-1H-imidazo[4,5-c]quinoline-1,4-diamine,1-[4-amino-2-ethyl-7-(pyridin-4-yl)-1H-imidazo[4,5-c]quinolin-1-yl]-2-methylpropan-2-ol,N-[4-(4-amino-2-ethyl-1H-imidazo[4,5-c]quinolin-1-yl)butyl]methanesulfonamide,orN-[4-(4-amino-2-butyl-1H-imidazo[4,5-c][1,5]naphthyridin-1-yl)butyl]-N′-cyclohexylurea such as described in WO2015103990A1. In this embodiment, the moiety linked to the antibody fragment is preferably a medicament for cancer. Moreover, the compound comprising the moiety linked to the antibody fragment is preferably a medicament for cancer.

In an embodiment, the moiety linked to the antibody fragment is a Pseudomonas exotoxin such as described in WO2015051199A2. In this embodiment, the moiety linked to the antibody fragment is preferably a medicament for cancer. Moreover, the compound comprising the moiety linked to the antibody fragment is preferably a medicament for cancer.

WO2017137553A1, WO2016038383A1, WO2018192944A1, WO2015051199A2, WO2017211809A1, WO2014057074A1, WO2015052322A1, WO2014140174A1, WO2015052321A1, WO2017186894A1, WO2017137555A1, WO2015162293A1, WO2015118030A2, WO2015103990A1, WO2017077382A1, WO2017134301A1, WO2017194783A1, WO2017194782A2, WO2018077893A1, WO2018141964A1, WO2018144999A1, WO2019032661A1, WO2019032663A1, WO2019032662A1, WO2019148089A1, WO2019191519A1 and WO2020033646A1 are incorporated in their entirety, and all compounds disclosed therein may be a moiety linked to the antibody fragment in the context of the current application.

Labelled Compound

In a further aspect, there is provided a labelled compound that comprises or consists of or essentially consists of an antibody fragment as defined in one of the previous aspects, preferably a heavy chain antibody (VHH) or a fragment thereof, which specifically binds human and/or murine FAP, wherein said antibody fragment is linked to a moiety which is a label.

In an embodiment, the label is a radionuclide (i.e. a radioactive label). Processes for labelling the antibody fragment to a radionuclide are disclosed in detail in the definition part at the end of the description. In a preferred embodiment, an antibody fragment, preferably a heavy chain antibody (VHH) or a fragment thereof, which specifically binds human and/or murine FAP is linked to a moiety and the moiety is a radionuclide.

In this aspect, the antibody fragment, preferably a heavy chain antibody (VHH) or a fragment thereof, which is coupled to a radionuclide may be called a labelled or a radiolabelled compound.

In an embodiment, such labelled compound fulfils at least one of the structural features earlier defined herein: first and/or second structural features and/or at least one of the functional features (i.e. specifically binds human and/or murine FAP, preferably human and murine FAP, and/or is not a modulator of FAP) defined herein.

Examples of suitable radionuclides which can be linked to the antibody fragment of the invention especially for therapeutic applications, preferably a VHH as disclosed herein can for example without any limitation be chosen from the group consisting of a-emitting radioisotopes and β⁻-emitting radioisotopes, including but not limited to a radioisotope chosen from the group consisting of Actinium-225, Astatine-211, Bismuth-212, Bismuth-213, Caesium-137, Chromium-51, Cobalt-60, Copper-67, Dysprosium-165, Erbium-169, Fermium-255, Gold-198, Holium-166, Iodine-125, Iodine-131, Iridium-192, Iron-59, Lead-212, Lutetium-177, Molybdenum-99, Palladium-103, Phosphorus-32, Potassium-42, Rhenium-186, Rhenium-188, Samarium-153, Radium-223, Radium-224, Ruthenium-106, Scandium-47, Sodium-24, Strontium-89, Terbium-149, Terbium-161, Terbium-149, Thorium-227, Xenon-133, Ytterbium-169, Ytterbium-177 and Yttrium-90.

In still further particular embodiments, the radionuclide present in the labelled compound as disclosed herein is Iodine-131.

In still further particular embodiments, the radionuclide present in the labelled compound as disclosed herein is Actinium-225.

In still further particular embodiments, the radionuclide present in the labelled compound as disclosed herein is Lutetium-177.

Examples of suitable radionuclides which can be linked to the antibody fragment of the invention especially for diagnostic applications, preferably a VHH as disclosed herein can for example without any limitation be chosen from the group consisting of positron-emitting radioisotopes (PET) or y-emitting radioisotopes (SPECT), including chosen from the group consisting of: Iodine-131, Yttrium-90, Iodine-125, Lutetium-177, Rhenium-186, Rhenium-188, Scandium-43, Scandium-44, Technetium-99m, Terbium-161, Indium-111, Xenon-133, Thallium-201, Fluorine-18, Gallium-68, Gallium-67, Copper-67, Iodine-123, Iodine-124, Zirconium-89 and Copper-64.

In still further particular embodiments, the radionuclide present in the labelled compound as disclosed herein is Iodine-131.

In still further particular embodiments, the radionuclide present in the labelled compound as disclosed herein is Actinium-225.

In still further particular embodiments, the radionuclide present in the labelled compound as disclosed herein is Lutetium-177.

Examples of suitable radionuclides which can be linked to the antibody fragment of the invention especially for theranostic (i.e. diagnostic and therapeutic) applications, preferably a VHH as disclosed herein can for example without any limitation be chosen from the group consisting of: Actinium-225, Bismuth-213, Iodine-125, Iodine-131, Lutetium-177, Yttrium-90, Copper-67, Rhenium-186, Rhenium-188 and Terbium-161.

In an embodiment, the linker separating the antibody fragment, preferably a heavy chain antibody (VHH) or a fragment thereof, is a benzoate linker. Preferably, this benzoate linker comprises N-succinimidyl-4-guanidinomethyl-3-[1-131]iodobenzoate (SGMIB) or a suitable derivative thereof. Alternatively, 2-[Bis[2-[bis(carboxymethyl)amino]ethyl]amino]acetic acid (DTPA), 1,4,7,10-Tetraazacyclododecane-1,4,7,10-tetraacetic acid (DOTA) or N,N′-bis[(6-carboxy-2-pyridil)methyl]-4,13-diaza-18-crown-6 (MACROPA) or a derivative thereof may be used.

In an embodiment, the labelled compound is:
the antibody fragment as defined earlier herein which is linked via SGMIB to Iodine-131,
the antibody fragment as defined earlier herein which is linked via DTPA or DOTA to Lutetium-177,
the antibody fragment as defined earlier herein which is linked via DOTA to Actinium-225 or
the antibody fragment as defined earlier herein which is linked to Technetium-99m.

Each of these labelled compounds had been synthesized and tested in the experimental part.

In a preferred embodiment, the labelled compound is:
an antibody fragment which specifically binds human and/or murine FAP, wherein the epitope is comprised within the amino acid stretch or region 65-90 and/or 101-140 of SEQ ID NO:26, said antibody fragment being linked via SGMIB to Iodine-131,
an antibody fragment which specifically binds human and/or murine FAP, wherein the epitope is comprised within the amino acid stretch or region 65-90 and/or 101-140 of SEQ ID NO:26, said antibody fragment being linked via DOTA or DTPA to Lutetium-177,
an antibody fragment which specifically binds human and/or murine FAP, wherein the epitope is comprised within the amino acid stretch or region 65-90 and/or 101-140 of SEQ ID NO:26, said antibody fragment being linked via DOTA to Actinium-225 or
an antibody fragment, which specifically binds human and/or murine FAP, wherein the epitope is comprised within the amino acid stretch or region 65-90 and/or 101-140 of SEQ ID NO:26, said antibody fragment being linked to Technetium-99m.

In a preferred embodiment, the labelled compound is:
an antibody fragment, which specifically binds human and/or murine FAP, wherein the conformational epitope is comprised within the combination of amino acid stretch or region 65-90 and 101-140 of SEQ ID NO:26, said antibody fragment being linked via SGMIB to Iodine-131,
an antibody fragment which specifically binds human and/or murine FAP, wherein the conformational epitope is comprised within the combination of amino acid stretch or region 65-90 and 101-140 of SEQ ID NO:26, said antibody fragment being linked via DOTA or DTPA to Lutetium-177,
an antibody fragment which specifically binds human and/or murine FAP, wherein the conformational epitope is comprised within the combination of amino acid stretch or region 65-90 and 101-140 of SEQ ID NO:26, said antibody fragment being linked via DOTA to Actinium-225 or
an antibody fragment, which specifically binds human and/or murine FAP, wherein the conformational epitope is comprised within the combination of amino acid stretch or region 65-90 and 101-140 of SEQ ID NO:26, said antibody fragment being linked to Technetium-99m.

In a preferred embodiment, the labelled compound is:
an antibody fragment, which specifically binds human and/or murine FAP, wherein at least one of amino acids I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and/or Y458 of SEQ ID NO:26 interacts with said antibody fragment, said antibody fragment being linked via SGMIB to Iodine-131,
an antibody fragment, which specifically binds human and/or murine FAP, wherein at least one of amino acids I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and/or Y458 of SEQ ID NO:26 interacts with said antibody fragment, said antibody fragment being linked via DOTA or DTPA to Lutetium-177,
an antibody fragment which specifically binds human and/or murine FAP, at least one of amino acids I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and/or Y458 of SEQ ID NO:26 interacts with said antibody fragment, said antibody fragment being linked via DOTA to Actinium-225 or
an antibody fragment, which specifically binds human and/or murine FAP, at least one of amino acids I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and/or Y458 of SEQ ID NO:26 interacts with said antibody fragment, said antibody fragment being linked to Technetium-99m.

In a preferred embodiment, the labelled compound is:
an antibody fragment that specifically binds human and/or murine FAP, wherein said antibody fragment is represented by an amino acid sequence having at least 80% sequence identity (or similarity) with at least one of SEQ ID NO:1, 2, 3, 4 or a portion thereof, said antibody fragment being linked via SGMIB to Iodine-131,
an antibody fragment that specifically binds human and/or murine FAP, wherein said antibody fragment is represented by an amino acid sequence having at least 80% sequence identity (or similarity) with at least one of SEQ ID NO:1, 2, 3, 4 or a portion thereof, said antibody fragment being linked via DOTA or DTPA to Lutetium-177,
an antibody fragment that specifically binds human and/or murine FAP, wherein said antibody fragment is represented by an amino acid sequence having at least 80% sequence identity (or similarity) with at least one of SEQ ID NO:1, 2, 3, 4 or a portion thereof, said antibody fragment being linked via DOTA to Actinium-225 or,
an antibody fragment that specifically binds human and/or murine FAP, wherein said antibody fragment is represented by an amino acid sequence having at least 80% sequence identity (or similarity) with at least one of SEQ ID NO:1, 2, 3, 4 or a portion thereof said antibody fragment being linked to Technetium-99m.

In an embodiment, the sequence identity (or similarity) with this sequence is at least 81%, 82%, 83%, 84%, 85%, 86%, 87%, 88%, 89%, 90%, 91%, 92%, 93%, 94%, 95%, 96%, 97%, 98%, 99% or 100%. Portion thereof has already been defined herein.

Each of the other embodiment relating to the first, second, third or fourth structural feature and/or functional features (i.e. specifically bind human and/or murine FAP, preferably which is to specifically bind human and murine FAP and which is not to modulate a FAP activity) may be combined to further define the labelled compound of the invention.

The labelled compound is specifically directed against human and/or murine FAP which is considered as a cancer antigen. The labelled compound may be used as a diagnostic molecule and/or as a therapeutic molecule. In an embodiment, the disease diagnosed or treated is cancer.

As used herein, human FAP is considered a ‘cancer cell-specific antigen’, ‘cancer-specific antigen’, ‘cancer antigen’, ‘target protein present on, ‘target protein expressed in’ and/or ‘specific for a cancer cell’, ‘cancer cell-specific target (protein)’, ‘cancer (cell)-associated antigen’ are used interchangeably herein and refers to the fact that human FAP is mainly present on (or mainly expressed on) cancer cells and in the vicinity of a tumor and/or in the vicinity of metastases. FAP is specifically expressed and more specifically overexpressed in cancer-associated fibroblasts (CAF) which have a tumorigenic function. It is also expressed in some cancer cells (such as leukemia, bone, uterus, pancreas, skin, muscle, brain, breast, colorectal, esophageal, gastric, liver, lung, ovarian, parathyroid, renal cancer (Puré et al 2018, Oncogene August; 37(32):4343-4357, as disclosed later herein). FAP is poorly expressed in healthy cells. Human FAP may therefore be considered as a tumour antigen or a cancer cell antigen and may therefore be used as diagnostic and/or therapeutic target.

For example, human FAP is expressed in cancer-associated fibroblast. As used herein, the term “FAP positive” or “expressing FAP” or “overexpressing FAP” may refer to cancerous or malignant human cells and/or cancer-associated fibroblasts or tissue characterized by FAP protein overexpression and thus have abnormally high levels of the FAP gene and/or the FAP protein compared to normal healthy cells. In this context, “overexpressing” may mean that the expression is at least 10%, 20%, 30%, 40%, 50%, 60%, 70%, 80%, 90%, 100% or more than the expression in a control cell line. A control cell line may be a healthy or non-diseased cell.

In another embodiment, the label present in the labelled compound is a non-radioactive label. In an embodiment, such non-radioactive label is a fluorescent label. This non-radioactive labelled compound may be used for diagnostic applications as defined herein. Alternative applications include image-guided surgery or photodynamic therapy. Examples of suitable fluorescent labels for diagnostic applications include Alexa fluor variants, Cy3, Cy5, FITC (fluorescein), Coumarin, Texas red, Oregon Green, Pacific Blue, Pacific Green, Pacific Orange, PE-Cyanine7, PerCP-Cyanine5.5, TRITC (tatramethylrhodamine). Examples of suitable fluorescent labels for image-guided surgery include IRDye800CW, IRDye680-RD, ZW800-1, FNIR (see for example Pieterjan Debie et al, Front Pharmacology, 2019; 10:510, doi: 10.3389/fphar.2019.00510, PMCID: PMC6527780 PMID: 31139085). Example of a suitable fluorescent label for photodynamic therapy includes IRDye700DX. Most labels may be obtained from ThermoFisher or from Licor.

Composition

In a further aspect there is provided a composition comprising or consisting essentially of an antibody fragment, such as a VHH or a fragment thereof. In a further aspect, there is also provided a composition comprising or consisting essentially of a compound, when said antibody fragment is linked to an entity such as a moiety. The composition comprises an excipient. The excipient should be acceptable for diagnostic and/or therapeutic purpose. In an embodiment the composition is a pharmaceutical composition. In another embodiment, the composition is a diagnostic composition. It is also encompassed by the invention that the composition is a pharmaceutical and diagnostic composition. Suitable formulations of the invention are disclosed in the definition part at the end of the description. A diagnostic composition as defined herein may comprise a screening dose or a stratification dose and may therefore be called a screening composition or a stratification composition.

The pharmaceutical compositions as envisaged herein can be used in the prevention and/or treatment of diseases and disorders associated with human FAP. In an embodiment, the disease is a cancer associated with expression or overexpression of human FAP. In particular, the application provides pharmaceutical compositions that are suitable for prophylactic and/or therapeutic use in a warm-blooded animal, and in particular in a mammal, and more in particular in a human being.

Kit

In a further aspect there is also provided a kit. Such as a kit is suitable for diagnostic and therapeutic applications as described herein. Such applications include the use of the antibody fragment of the invention, of the compound of the invention comprising the antibody fragment linked to an entity such as a moiety, said moiety being a radioactive or a non-radioactive label. A more detailed definition of the kit is provided in the part dedicated to the definition at the end of the description.

Diagnostic Use of the Antibody Fragment and of the Labelled Compound

In a further aspect, there is provided a method wherein the antibody fragment or the labelled compound or a composition comprising it is used to assess expression of human FAP in a subject or in an isolated sample of said subject. This method may comprise the following steps:
a) providing an antibody fragment or a labelled compound as identified herein,
b) administering it to a subject or contacting it with an isolated sample of a subject,
c) assessing the expression of human FAP in said subject or in said isolated sample of said subject.

This method may be called a diagnostic method. This method may be an in vitro or an in vivo method. This method may allow the localization of the expression of human FAP in a subject or in an isolated sample of said subject and may allow the prediction and/or prognosis of a certain disease and/or disorder and/or condition in said subject. In an embodiment, this method may be a stratification method to identify patients that are likely to respond to a particular treatment such as cancer treatment or wound healing treatment or fibrosis treatment. Therefore, in a further aspect, there is provided a method wherein the antibody fragment or the labeled compound or a composition comprising it is used to stratify the subject and assess whether the subject will be likely to respond to a particular treatment such as cancer treatment or wound healing treatment or fibrosis treatment. This method may comprise the following steps:
a) providing an antibody fragment or a labelled compound as identified herein,
b) administering it to a subject or to an isolated sample of a subject,
c) assessing the expression of human FAP in said subject or in said isolate sample of said subject and
d) deciding whether the subject is likely to be responsive to a medical treatment such as the one comprising the labelled compound of the invention.

The antibody fragment of the invention may be used in such a diagnostic method. The antibody fragment of the invention does not per se need to be coupled to a label in order to be used in such a method. Such a method may be an ELISA.

Optionally if the method defined above is carried out using a radioactive labelled compound, the radioactive labelled compound or a version thereof suited for therapy is administrated to the subject as a treatment. The subject is preferably a human being. Each and every radioactive labelled compound as defined earlier herein is suitable in this method. Detailed information is disclosed in the definition part at the end of the description in order to produce/provide and in order to administer a labelled compound as identified herein. The administration of a labelled compound for diagnostic purpose and for therapeutic purpose is similar. A method according to this aspect may be an in vitro, ex vivo method.

In an embodiment, a screening dose or a biomarker dose is administered to a subject or to an isolated sample of said subject. Detailed definitions are provided later on especially by comparison to the definition of a therapeutic dose.

In an embodiment of the diagnostic method, the labeled compound is
the antibody fragment as defined earlier herein which is linked via SGMIB to Iodine-131,
the antibody fragment as defined earlier herein which is linked via DTPA or DOTA to Lutetium-177 or
the antibody fragment as defined earlier herein which is linked to Technetium-99m.

Each of these labelled compounds had been synthesized and tested in the experimental part.

The assessment of the expression of human FAP in the subject is preferably carried out using imaging as disclosed in the part dedicated to definition at the end of the description. Alternatively, the assessment of the expression of human FAP in the subject is preferably carried out using an isolated sample of the subject. Within the context of the invention, an isolated sample of a subject may be a tissue or a liquid sample from said subject. A liquid may be serum. An isolated sample from a patient may be called a biopsy or a tumor biopsy.

Therapeutic Use of the (Labelled) Compound

In a further aspect there is provided an antibody fragment, preferably a VHH or a fragment thereof or a compound or a labelled compound or a composition (all as defined herein) for use as a medicament.

In an embodiment, the compound comprises an entity such as a moiety linked to the antibody fragment (preferably a VHH or a fragment thereof) and said moiety is a molecule to be delivered to a cell, a tissue, organ expressing or over-expressing FAP. The molecule may be a peptide or a small molecule, a nucleic acid. A peptide may be a cytokine. A small molecule may be a chemotherapeutic. An entity may be a cell such as a CAR-T cell, a CAR-NK cell, a BITE or a LITE. This compound or a composition comprising it may be a medicament for treating a disease or condition associated with the expression or the over-expression of FAP.

Any moiety, molecule or medicament known to act on a cell, tissue, organ expressing FAP is potentially encompassed by the present invention and could be linked to the antibody fragment of the invention.

In another embodiment, the compound is a labelled compound and said labelled compound or a composition comprising the same is a medicament for treating a cancer. In an embodiment, said cancer is associated with an expression of human FAP on a cancer or a tumour cell or a metastasized lesion. The cancer treated may be metastatic, preferably wherein a metastatic cell is found in the brain, bones, liver, lung. A cancer associated with expression of FAP may be any of a leukemia, bone, uterus, pancreas, GEP-NET (gastroenteropancreatic neuroendocrine tumor), skin, muscle, brain, breast, colorectal, esophageal, gastric, liver, lung, NSCLC (non small cell lung cancer) ovarian, parathyroid, renal cancer cells, CUP (cancer of unknown primary), prostate, small intestine, CCC (Cholangiocellular Carcinoma), sarcoma, (Puré et al 2018, Oncogene August; 37(32):4343-4357 and Frederik Giesel et al J Nucl Med May 1, 2019 vol. 60 no. supplement 1 Abstract 289) However, the invention is not limited to these types of cancer. As soon as a subject is suspected to have a cancer cell expressing or overexpressing human FAP, the labelled compound or a composition comprising the same may be used.

In an embodiment, the subject has been first diagnosed using a labelled compound of the invention before being treated with the same or with a distinct label compound. The identity of the nuclide may not be the same in diagnostic and therapy applications.

In an embodiment of this therapeutic method or use, the labelled compound is:
the antibody fragment as defined earlier herein which is linked via SGMIB to Iodine-131,
the antibody fragment as defined earlier herein which is linked via DTPA or DOTA to Lutetium-177 or
the antibody fragment as defined earlier herein which is linked via DOTA to Actinium-225.

Each of these labelled compounds had been synthesized and tested in the experimental part.

Within the context of the invention, a disease or condition or disorder has been prevented or treated when the administration of a compound respectively a labelled compound has been carried out and has resulted:
in the improvement of at least one symptom associated with said disease or condition or disorder and/or
in the improvement of at least one parameter associated with said disease or condition or disorder.

The improvement may be observed at least one day, two days, three days, four days, five days, six days, one week after the compound, respective labelled compound has been administrated. Alternatively, the improvement may be observed at least one month, six months after the administration of the compound, respectively the labelled compound. Envisaged doses and administration modes are further disclosed in the definition part at the end of the description.

A labelled compound or a composition comprising the same exhibits an anti-cancer activity when at least one of the following is fulfilled:
it can kill a tumour cell, a cancer cell and/or a CAF that expresses human FAP,
It can reduce or slow the growth and/or proliferation of such a tumour cell or cancer cell.
It can reduce the size of a primary tumour or of a metastatic lesion,
It can delay the occurrence of metastases and/or of tumour cell migration,
It can delay the increase of a tumour weight or growth, and
It can extend patient survival of at least one month, several months or more (compared to those not treated or treated with a control or compared with the subject at the onset of the treatment).

An anti-cancer activity may have been identified or determined when the number of viable cancer cells, and/or viable tumor cells after the administration of the labelled compound is less than 90%, less than 80%, less than 70%, less than 60%, less than 50%, less than 40%, less than 30%, less than 20%, less than 10% of the number of initial viable cancer cells and/or initial viable tumor cells.

An anti-cancer activity may have been identified or determined when the size of a primary tumor and/or the size of a metastatic lesion after the administration of the labelled compound is less than 90%, less than 80%, less than 70%, less than 60%, less than 50%, less than 40%, less than 30%, less than 20%, less than 10% of the size of said primary tumor and/or of the size of said metastatic lesion.

Tumor cell death may be assessed by measurement of radiolabeled Annexin A5, a molecular imaging agent to measure cell death in vitro, and non-invasively in patients with cancer such as ICH (Schutters K. et al., Apoptosis 2010; de Saint-Hubert M. et al., Methods 48: 178, 2009). ICH has been defined in the definition part at the end of the description.

Tumor growth may be inhibited at least 5%, 10%, 20%, 30%, 40%, 50%, 55%, 60%, 65%, 70% or 75%, or more. Tumor growth may be assessed using techniques known to the skilled person. Tumor growth may be assessed using MRI (Magnetic Resonance Imaging) or CT (Computer Tomography). In certain embodiments, tumor weight increase or tumor growth may be inhibited at least 20%, 30%, 40%, 50%, 55%, 60%, 65%, 70% or 75%, or more. Tumor weight or tumor growth may be assessed using techniques known to the skilled person. The detection of tumor growth or the detection of the proliferation of tumor cells may be assessed in vivo by measuring changes in glucose utilization by positron emission tomography with the glucose analogue 2-[18F]-fluor-2-deoxy-D-glucose (FDG-PET) or [18F]-′3-fluoro-′3-deoxy-L-thymidine (FLT-PET). An ex vivo alternative may be staining of a tumor biopsy with Ki67.

A delay in occurrence of metastases and/or of tumor cell migration may be a delay of at least one week, one month, several months, one year or longer. The presence of metastases may be assessed using MRI, CT or Echography or techniques allowing the detection of circulating tumour cells (CTC). Examples of the latter tests are CellSearch CTC test (Veridex), an EpCam-based magnetic sorting of CTCs from peripheral blood.

In certain embodiments, tumor growth may be delayed or inhibited at least one day, two days, three days, four days, five days, six days or one week, two weeks, three weeks, one month, two months or more. In a certain embodiment, an occurrence of metastases is delayed at least one week, two weeks, three weeks, four weeks, one months, two months, three months, four months, five months, six months or more.

The labelled compound of the invention exerts its anti-cancer activity through the mechanism of radiotoxicity once it is bound to a cancer or tumour cell or metastatic lesion or CAF expressing human FAP.

Tumour cell, cancer cell, lesion, metastatic lesion and dose of the labelled compound have been defined in the section entitled definition.

In a further aspect, there is provided a method for the prevention and/or treatment of a disease and/or disorder and/or condition comprising administering to a subject in need thereof, an antibody fragment, preferably a VHH or a fragment thereof or a compound or a labelled compound or a composition as envisaged herein. All features of this method have been defined earlier herein.

Non-Human Mammal

In a further aspect, there is provided a non-human animal comprising a nucleic acid construct allowing the expression of human FAP. A non-human animal may be a mammal. Preferred mammals include mouse, rat, rabbit. Such animal may be obtained using common knowledge techniques known to the skilled person. In an embodiment, such non-human animal may have been modified to no longer express its endogenous FAP. In an embodiment, the endogenous FAP of the non-human animal has been replaced by the human FAP gene. The human FAP coding nucleic acid is represented by SEQ ID NO: 25. This gene replacement may be carried out by homologous recombination as known to the skilled person. In an embodiment, the targeting vector used comprises SEQ ID NO: 27. In a preferred embodiment, the non-human animal is a mouse and the targeting vector comprising SEQ ID NO: 27 has been introduced into it using techniques known to the skilled person. The resulting mouse does no longer express murine FAP and instead thereof expresses human FAP. In an embodiment, the mouse has been obtained as described in example 2. In an embodiment, the expression of human FAP is assessed in said non-human animal by the labeled compound of the invention. Alternatively, it can be assessed using other antibodies or antibody fragments known to be specific for human FAP, such as commercial mouse anti-human Fibroblast Activation Protein alpha APC-conjugated Antibody (R&D Systems, FAB3715A). Once the expression of human FAP has been validated in this non-human animal, it can be used to assess the functionality of an antibody fragment, a compound or of a labeled compound of the invention. This non-human animal may therefore be used in a method for screening a molecule specifically binding human FAP, preferably a new antibody fragment or a compound of the invention.

### Definitions

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2^{nd} ed., Cold Spring Harbor Press, Plainsview, New York (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms ‘a’, ‘an’, and ‘the’ include both singular and plural referents unless the context clearly dictates otherwise.

The terms ‘comprising’, ‘comprises’ and ‘comprised of’ as used herein are synonymous with ‘including’, ‘includes’ or ‘containing’, ‘contains’, and are inclusive or open-ended and do not exclude additional, non-recited members, elements or method steps. The expression “essentially consists of” used in the context of a product or a composition (“a product essentially consisting of” or “a composition essentially consisting of”) means that additional molecules may be present but that such molecule does not change/alter the characteristic/activity/functionality of said product or composition. For example, a composition may essentially consist of an antibody fragment if the composition as such would exhibit similar characteristic/activity/functionality as one of the antibody fragments.

The recitation of numerical ranges by endpoints includes all numbers and fractions subsumed within the respective ranges, as well as the recited endpoints.

The term ‘about’ as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

Polypeptide/Nucleic Acid Molecule and Identity/Similarity

As used herein, amino acid residues will be indicated either by their full name or according to the standard three-letter or one-letter amino acid code.

As used herein, the terms ‘polypeptide’ or ‘protein’ are used interchangeably, and refer to a polymeric form of amino acids of any length, which can include coded and non-coded amino acids, chemically or biochemically modified or derivatized amino acids, and polypeptides having modified peptide backbones. A “peptide” is also a polymer of amino acids with a length which is usually of up to 50 amino acids. A polypeptide or peptide is represented by an amino acid sequence.

As used herein, the terms ‘nucleic acid molecule’, ‘polynucleotide’, ‘polynucleic acid’, ‘nucleic acid’ are used interchangeably and refer to a polymeric form of nucleotides of any length, either deoxyribonucleotides or ribonucleotides, or analogs thereof. A nucleic acid molecule is represented by a nucleic acid sequence, which is primarily characterized by its base sequence. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term ‘homology’ denotes at least secondary structural identity or similarity between two macromolecules, particularly between two polypeptides or polynucleotides, from same or different taxons, wherein said similarity is due to shared ancestry. Hence, the term ‘homologues’ denotes so-related macromolecules having said secondary and optionally tertiary structural similarity. For comparing two or more nucleotide sequences, the ‘(percentage of) sequence identity’ between a first nucleotide sequence and a second nucleotide sequence may be calculated using methods known by the person skilled in the art, e.g. by dividing the number of nucleotides in the first nucleotide sequence that are identical to the nucleotides at the corresponding positions in the second nucleotide sequence by the total number of nucleotides in the first nucleotide sequence and multiplying by 100% or by using a known computer algorithm for sequence alignment such as NCBI Blast. In determining the degree of sequence similarity between two amino acid sequences, the skilled person may take into account so-called ‘conservative’ amino acid substitutions, which can generally be described as amino acid substitutions in which an amino acid residue is replaced with another amino acid residue of similar chemical structure and which has little or essentially no influence on the function, activity or other biological properties of the polypeptide. Possible conservative amino acid substitutions will be clear to the person skilled in the art. Amino acid sequences and nucleic acid sequences are said to be ‘exactly the same’ if they have 100% sequence identity over their entire length.

Throughout this application, each time one refers to a specific amino acid sequence SEQ ID NO (take SEQ ID NO: Y as example), one may replace it by: a polypeptide comprising an amino acid sequence that has at least 80% sequence identity or similarity with amino acid sequence SEQ ID NO: Y.

Each amino acid sequence described herein by virtue of its identity percentage (at least 80%) with a given amino acid sequence respectively has in a further preferred embodiment an identity of at least 80%, 85%, 90%, 95%, 97%, 98%, 99% or more identity with the given amino acid sequence respectively. In a preferred embodiment, sequence identity is determined by comparing the whole length of the sequences as identified herein. Each amino acid sequence described herein by virtue of its similarity percentage (at least 81%) with a given amino acid sequence respectively has in a further preferred embodiment a similarity of at least 81%, 85%, 90%, 95%, 97%, 98%, 99% or more similarity with the given amino acid sequence respectively. In a preferred embodiment, sequence similarity is determined by comparing the whole length of the sequences as identified herein. Unless otherwise indicated herein, identity or similarity with a given SEQ ID NO means identity or similarity based on the full length of said sequence (i.e. over its whole length or as a whole).

“Sequence identity” is herein defined as a relationship between two or more amino acid (polypeptide or protein) sequences or two or more nucleic acid (polynucleotide) sequences, as determined by comparing the sequences. The identity between two amino acid sequences is preferably defined by assessing their identity within a whole SEQ ID NO as identified herein or part thereof. Part thereof may mean at least 50% of the length of the SEQ ID NO, or at least 60%, or at least 70%, or at least 80%, or at least 90%.

In the art, “identity” also means the degree of sequence relatedness between amino acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., NucleicAcids Research 12 (1): 387 (1984)), BestFit, FASTA, BLASTN, and BLASTP (Altschul, S. F. et al., J. Mol. Biol. 215:403-410 (1990)), EMBOSS Needle (Madeira, F., et al., Nucleic Acids Research 47(W1): W636-W641 (2019)). The BLAST program is publicly available from NCBI and other sources (BLAST Manual, Altschul, S., et al., NCBI NLM NIH Bethesda, MD 20894; Altschul, S., et al., J. Mol. Biol. 215:403-410 (1990)). The EMBOSS program is publicly available from EMBL-EBI. The well-known Smith Waterman algorithm may also be used to determine identity. The EMBOSS Needle program is the preferred program used.

Preferred parameters for polypeptide sequence comparison include the following: Algorithm: Needleman and Wunsch, J. Mol. Biol. 48 (3):443-453 (1970); Comparison matrix: BLOSUM62 from Henikoff and Henikoff, Proc. Natl. Acad. Sci. USA. 89:10915-10919 (1992); Gap Open Penalty: 10; and Gap Extend Penalty: 0.5. A program useful with these parameters is publicly available as the EMBOSS Needle program from EMBL-EBI. The aforementioned parameters are the default parameters for a Global Pairwise Sequence alignment of proteins (along with no penalty for end gaps).

Preferred parameters for nucleic acid comparison include the following: Algorithm: Needleman and Wunsch, J. Mol. Biol. 48:443-453 (1970); Comparison matrix: DNAfull; Gap Open Penalty: 10; Gap Extend Penalty: 0.5. A program useful with these parameters is publicly available as the EMBOSS Needle program from EMBL-EBI. The aforementioned parameters are the default parameters for a Global Pairwise Sequence alignment of nucleotide sequences (along with no penalty for end gaps).

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; a group of amino acids having acidic side chains is aspartate and glutamate; and a group of amino acids having sulphur-containing side chains is cysteine and methionine. Preferred conservative substitutions for each of the naturally occurring amino acids are as follows: Ala to Ser; Arg to Lys or Gln; Asn to Asp, His or Ser; Asp to Glu or Asn; Gln to Glu, Lys or Arg; Glu to Lys, Asp, Gln; His to Tyr or Asn; lie to Leu, Val, or Met; Leu to lie, Met or Val; Lys to Arg, Gln or Glu; Met to Val, Leu or lie; Phe to Trp or Tyr; Ser to Thr, Ala or Asn; Thr to Ser; Trp to Tyr or Phe; Tyr to His, Trp or Phe; and Val to lie, Leu or Met. Substitutional variants of the amino acid sequence disclosed herein are those in which at least one residue in the disclosed sequences has been removed and a different residue inserted in its place. Preferably, the amino acid change is conservative.

Method for Isolating Suitable Antibody Fragment Against Human Fap

In particular embodiments, antibody fragment, preferably VHH or fragment thereof disclosed herein are obtained by affinity selection against human and/or murine FAP present on and/or specific for a solid tumor and/or a cancer cell. Obtaining suitable polypeptides by affinity selection against a particular solid tumor antigen or cancer cell may for example be performed by screening a set, collection or library of cells that express antibody fragment, preferably VHH's on their surface (e.g. bacteriophages) for binding against a tumor-specific antigen and/or a cancer cell-specific antigen; all of which may be performed in a manner known per se, essentially comprising the following non-limiting steps: a) obtaining an isolated solution or suspension of a tumor-specific or cancer cell-specific protein target molecule, which molecule is known to be a target for a potential cancer drug; b) bio-panning phages or other cells from a VHH library against said protein target molecule; c) isolating the phages or other cells binding to the tumor-specific or cancer cell-specific protein target molecule; d) determining the nucleotide sequence encoding the VHH insert from individual binding phages or other cells; e) producing an amount of VHH according to this sequence using recombinant protein expression; D determining the affinity of said VHH domain for said tumor-specific or cancer cell-specific protein target molecule; and optionally g) testing the tumoricidal or anti-cancer activity of said VHH domain in a bio-assay. Various methods may be used to determine the affinity between the VHH domain and the tumor-specific or cancer cell-specific protein target molecule, including for example, enzyme linked immunosorbent assays (ELISA) or Surface Plasmon Resonance (SPR) assays, which are common practice in the art, for example, as described in Sambrook et al. (2001), Molecular Cloning, A Laboratory Manual. Third Edition. Cold Spring Harbor Laboratory Press, Cold Spring Harbor, NY. The equilibrium dissociation constant is commonly used to describe the affinity between a polypeptide and its target molecule. Typically, the equilibrium dissociation constant is lower than 10⁻⁷ M. Preferably, the equilibrium dissociation constant is lower than 10⁻⁸ M, or lower than 10⁻⁹ M, or more preferably, ranged from 10⁻⁹ M to 10⁻¹² M.

Antibody

As used herein, the term ‘antibody’ refers to polyclonal antibodies, monoclonal antibodies, humanized antibodies, single-chain antibodies, and fragments thereof such as Fab F(ab′)₂, scFv, VHH and other fragments that retain the antigen binding function of the parent antibody. As such, an antibody may refer to an immunoglobulin or glycoprotein, or fragment or portion thereof, or to a construct comprising an antigen-binding portion comprised within a modified immunoglobulin-like framework, or to an antigen-binding portion comprised within a construct comprising a non-immunoglobulin-like framework or scaffold.

As used herein, the term ‘monoclonal antibody’ refers to an antibody composition having a homogeneous antibody population. The term is not limited regarding the species or source of the antibody, nor is it intended to be limited by the manner in which it is made. The term encompasses whole immunoglobulins as well as fragments and others that retain the antigen binding function of the antibody. Monoclonal antibodies of any mammalian species can be used in this invention. In practice, however, the antibodies will typically be of rat or murine origin because of the availability of rat or murine cell lines for use in making the required hybrid cell lines or hybridomas to produce monoclonal antibodies.

As used herein, the term ‘polyclonal antibody’ refers to an antibody composition having a heterogeneous antibody population. Polyclonal antibodies are often derived from the pooled serum from immunized animals or from selected humans.

‘Heavy chain variable domain of an antibody or a fragment thereof’, as used herein, means (i) the variable domain of the heavy chain of a heavy chain antibody, which is naturally devoid of light chains (also indicated hereafter as VHH), including but not limited to the variable domain of the heavy chain of heavy-chain antibodies of camelids or sharks or (ii) the variable domain of the heavy chain of a conventional four-chain antibody (also indicated hereafter as V_{H}), including but not limited to a camelized (as further defined herein) variable domain of the heavy chain of a conventional four-chain antibody (also indicated hereafter as camelized V_{H}) or any fragments thereof, such as but not limited to one or more stretches of amino acid residues (i.e. small peptides) that are particularly suited for binding to a tumor antigen or an antigen present on cancer cells and which are present in, and/or may be incorporated into, the VHH's as disclosed herein (or may be based on and/or derived from CDR sequences of the VHH's as disclosed herein). In an embodiment, the fragment of a VHH is a functional fragment.

As further described herein below, the amino acid sequence and structure of a heavy chain variable domain of an antibody can be considered, without however being limited thereto, to be comprised of four framework regions or ‘FR's’, which are referred to in the art and herein below as ‘framework region 1’ or ‘FR1’; as ‘framework region 2’ or ‘FR2’; as ‘framework region 3’ or ‘FR3’; and as ‘framework region 4’ or ‘FR4’, respectively, which framework regions are interrupted by three complementary determining regions or ‘CDR's’, which are referred to in the art as ‘complementarity determining region 1’ or ‘CDR1’; as ‘complementarity determining region 2’ or ‘CDR2’; and as ‘complementarity determining region 3’ or ‘CDR3’, respectively.

As used herein, the terms ‘complementarity determining region’ or ‘CDR’ within the context of antibodies refer to variable regions of either the H (heavy) or the L (light) chains (also abbreviated as VH and VL, respectively) and contain the amino acid sequences capable of specifically binding to antigenic targets. These CDR regions account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have 3 CDR regions, each non-contiguous with the others (termed L1, L2, L3, H1, H2, H3) for the respective light (L) and heavy (H) chains.

As also further described herein below, the total number of amino acid residues in a heavy chain variable domain of an antibody (including a VHH or a V_{H}) can be in the region of 110-130. It should however be noted that parts, fragments or analogs of a heavy chain variable domain of an antibody are not particularly limited as to their length and/or size, as long as such parts, fragments or analogs retain (at least part of) the functional activity, and/or retain (at least part of) the binding specificity of the original heavy chain variable domain of an antibody from which these parts, fragments or analogs are derived from. Parts, fragments or analogs retaining (at least part of) the functional activity, and/or retaining (at least part of) the binding specificity of the original heavy chain variable domain of an antibody from which these parts, fragments or analogs are derived from are also further referred to herein as ‘functional fragments’ of a heavy chain variable domain.

The amino acid residues of a variable domain of an antibody (including a VHH or a V_{H}) are preferably numbered according to the IMGT unique numbering for V-domain (immunoglobulins and T cell receptors) given by the IMGT nomenclature as described (Lefranc M. P. et al 1997 Immunology today, 18: 509, PMID: 9386342; Lefranc, M.-P., 1999 The Immunologist, 7: 132-136 and Lefranc M. P. et al 2003, Dev. Comp. Immunol., 27: 55-77 PMID: 12477501). According to this numbering (see for example table 1 of Lefranc 2003), the conserved amino acids always have the same position, for instance cysteine 23 (1st-CYS), tryptophan 41 (CONSERVED-TRP), hydrophobic amino acid 89, cysteine 104 (2nd-CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. Gaps represent unoccupied positions. Gaps in the CDR1-IMGT and CDR2-IMGT (less than 12 and 10 amino acid long, respectively) are put at the top of the CDR-IMGT loops. The basic length of a rearranged CDR3-IMGT is 13 amino acids (positions 105 to 117), which corresponds to a JUNCTION of 15 amino acids (2nd-CYS 104 to J-TRP or J-PHE 118). If the CDR3-IMGT length is less than 13 amino acids, gaps are created from the top of the loop, in the following order 111, 112, 110, 113, 109, 114, etc. If the CDR3-IMGT length is more than 13 amino acids, additional positions are created between positions 111 and 112 at the top of the CDR3-IMGT loop in the following order 112.1, 111.1, 112.2, 111.2, 112.3, 111.3, etc.

In this respect, it should be noted that—as is well known in the art for VHH domains—the total number of amino acid residues in each of the CDR's may vary and may not correspond to the total number of amino acid residues indicated by the IMGT numbering (that is, one or more positions according to the IMGT numbering may not be occupied in the actual sequence, or the actual sequence may contain more amino acid residues than the number allowed for by the IMGT numbering). This means that, generally, the numbering according to IMGT may or may not correspond to the actual numbering of the amino acid residues in the actual sequence.

Alternatively, the amino acid residues of a variable domain of an antibody (including a VHH or a VH) can be numbered according to Kabat numbering (Kabat et al 1987, National Institute of Health; 1987. 804 pp., Publication no. 165-462.). Correspondence between the IMGT and Kabat numbering for the immunoglobulin V-regions can be found for example in Table 2 of Lefranc et al., 2003.

For a general description of heavy chain antibodies and the variable domains thereof, reference is inter alia made to Muyldermans S., et al 2013 Annual Review of Biochemistry, 82: 775-797 as general background art.

Generally, it should be noted that the term ‘heavy chain variable domain’ as used herein in its broadest sense is not limited to a specific biological source or to a specific method of preparation. For example, as will be discussed in more detail below, the heavy chain variable domains derived from heavy chain antibodies (i.e. VHH's) as disclosed herein can be obtained (1) by isolating the VHH domain of a naturally occurring heavy chain antibody; (2) by expression of a nucleotide sequence encoding a naturally occurring VHH domain; (3) by ‘camelization’ (as described below) of a naturally occurring V_{H} domain from any animal species, in particular a species of mammal, such as from a human being, or by expression of a nucleic acid encoding such a camelized V_{H} domain; (4) by ‘camelization’ of a ‘domain antibody’ or ‘dAb’ as described by Weizao C., et al Methods Mol Biol 2009, 525:81-99)), or by expression of a nucleic acid encoding such a camelized V_{H} domain (5) using synthetic or semi-synthetic techniques for preparing proteins, polypeptides or other amino acid sequences; (6) by preparing a nucleic acid encoding a VHH using techniques for nucleic acid synthesis, followed by expression of the nucleic acid thus obtained; and/or (7) by any combination of the foregoing. Suitable methods and techniques for performing the foregoing will be clear to the skilled person based on the disclosure herein and for example include the methods and techniques described in more detail herein below.

An antibody fragment such as a single-domain antibody fragment as disclosed herein is considered to be ‘(in) essentially isolated (form)’ as used herein, when it has been extracted or purified from the host cell and/or medium in which it is produced.

Variant of Antibody Fragments

It should be noted that the invention is not limited as to the origin of the antibody fragment, preferably VHH sequences or fragments thereof of the invention (or of the nucleotide sequences of the invention used to express them). Furthermore, the present invention is also not limited as to the way that the antibody fragment, preferably VHH sequences or nucleotide sequences as disclosed herein have been generated or obtained. Thus, the amino acid sequences as disclosed herein may be synthetic or semi-synthetic amino acid sequences, polypeptides or proteins.

The present invention also encompasses parts, fragments, analogs, mutants, variants, and/or derivatives of the antibody fragment, preferably VHH specifically binding to human and/or murine FAP as disclosed herein and/or polypeptides comprising or essentially consisting of one or more of such parts, fragments, analogs, mutants, variants, and/or derivatives, as long as these parts, fragments, analogs, mutants, variants, and/or derivatives are suitable for the purposes envisaged herein: deliver to a cell, a tissue or an organ expressing FAP a molecule linked to it and suitable to be used in diagnostic and therapeutic applications when linked to a radionuclide. Such parts, fragments, analogs, mutants, variants, and/or derivatives according to the invention:
specifically bind to human and/or murine FAP, preferably to both FAP,
are preferably not modulators of FAP, preferably not inhibitors of FAP.

For example, the invention provides a number of stretches of amino acid residues (i.e. small peptides), also referred to herein as CDR sequences or part of the antibody fragment and identified as SEQ ID NO: 1, 2, 3, such as sequences having at least 80% identity with SEQ ID NO: 4 representing the sequence of the antibody fragment or a portion thereof, preferably VHH's as disclosed herein, that are particularly suited for binding to human and/or murine FAP.

For example, the invention provides a number of stretches of amino acid residues (i.e. small peptides), also referred to herein as CDR sequences or part of the antibody fragment and identified as SEQ ID NO: 5, 6, 7, such as sequences having at least 80% identity with SEQ ID NO: 8 representing the sequence of the antibody fragment or a portion thereof, preferably VHH's as disclosed herein, that are particularly suited for binding to human and/or murine FAP.

For example, the invention provides a number of stretches of amino acid residues (i.e. small peptides), also referred to herein as CDR sequences or part of the antibody fragment and identified as SEQ ID NO:9, 10, 11, such as sequences having at least 80% identity with SEQ ID NO: 12 representing the sequence of the antibody fragment or a portion thereof, preferably VHH's as disclosed herein, that are particularly suited for binding to human and/or murine FAP.

For example, the invention provides a number of stretches of amino acid residues (i.e. small peptides), also referred to herein as CDR sequences or part of the antibody fragment and identified as SEQ ID NO:13, 14, 15, such as sequences having at least 80% identity with SEQ ID NO: 16 representing the sequence of the antibody fragment or a portion thereof, preferably VHH's as disclosed herein, that are particularly suited for binding to human and/or murine FAP.

These stretches may be regarded as being functional fragments of the antibody fragment preferably VHH's as disclosed herein and may be present in, and/or may be incorporated into any suitable scaffold (protein), such as but not limited to the VHH's or compound or labelled compounds as disclosed herein, in particular in such a way that they form (part of) the antigen binding site of that suitable scaffold or VHH. It should however be noted that the invention in its broadest sense is not limited to a specific structural role or function that these stretches of amino acid residues may have in the scaffolds or antibody fragment, preferably VHH's as disclosed herein, as long as these stretches of amino acid residues allow these scaffolds or antibody fragment (preferably VHH's) as disclosed herein to specifically bind to human and/or murine FAP.

Further Posttranslational Structural Characterization of the Antibody Fragment

In certain aspects, the antibody fragment, preferably VHH domains or fragments thereof specifically binding to human and/or murine FAP as disclosed herein may be optionally linked to one or more further groups, moieties, or residues via one or more linkers. These one or more further groups, moieties or residues can serve for binding to other targets of interest. It should be clear that such further groups, residues, moieties and/or binding sites may or may not provide further functionality to the antibody fragment as disclosed herein and may or may not modify its properties as disclosed herein. Such groups, residues, moieties or binding units may also for example be chemical groups which can be biologically active.

These groups, moieties or residues are, in particular embodiments, linked N- or C-terminally to the heavy chain variable domain, in particularly C-terminally linked.

In particular embodiments, the antibody fragment, preferably VHH domains or fragments thereof specifically binding to human and/or murine FAP antigen as disclosed herein may also have been chemically modified. For example, such a modification may involve the introduction or linkage of one or more functional groups, residues or moieties into or onto the antibody fragment, preferably VHH domain. These groups, residues or moieties may confer one or more desired properties or functionalities to the antibody fragment, preferably VHH domain. Examples of such functional groups will be clear to the skilled person.

For example, the introduction or linkage of such functional groups to antibody fragment, preferably VHH domains or fragments thereof can result in an increase in their solubility and/or their stability, in a reduction of their toxicity, or in the elimination or attenuation of any undesirable side effects, and/or in other advantageous properties.

In particular embodiments, one or more groups, residues or moieties are linked to the antibody fragment, preferably VHH domains or fragments thereof via one or more suitable linkers or spacers.

In cases where all of the two or more binding sites of an antibody fragment such as a VHH or fragments thereof, as disclosed herein are directed against or specifically bind to the same site, determinant, part, epitope, domain or stretch of amino acid residues of the human and/or murine FAP, the antibody fragment as disclosed herein is said to be ‘bivalent’ (in the case of two binding sites on the single-domain antibody fragment) or multivalent (in the case of more than two binding sites on the single-domain antibody fragment), such as for example trivalent.

In an embodiment, the antibody fragment, preferably VHH or fragment thereof is present in a monovalent format.

As used herein, the term ‘monovalent’ when referring to an antibody fragment, such as a VHH or fragments thereof, denotes an antibody fragment in monomeric form. A monovalent antibody fragment contains only one binding site. In this context, the binding site of an antibody fragment, such as a VHH or fragments thereof, encompasses one or more ‘complementarity determining regions’ or ‘CDRs’ represented by SEQ ID NO:1, 2 and/or 3 and/or one or more regions identified herein as having at least 80% identity with SEQ ID NO: 4 of an antibody fragment that are directed against or specifically bind to a particular site, determinant, part, epitope, domain or stretch of amino acid residues of human and/or murine FAP.

In particularly preferred embodiments, the present invention provides an antibody fragment, preferably a VHH or a fragment thereof in its monomeric form, i.e. comprising only one VHH domain. The small size of such molecule is attractive for therapeutic/diagnostic applications. For specific applications, such a small size may also be attractive if a high tissue penetration is needed in order to reach an optimal therapeutic effect.

In alternative embodiments, however the present invention also provides an antibody fragment, preferably a VHH or a fragment comprising two or more identical or different VHH domains resulting in a bivalent (or multivalent) or a bispecific or (multispecific) polypeptide.

While the antibody fragment, preferably a VHH or a fragment thereof may be present in its monomeric form, in particular alternative embodiments, two or more of the antibody fragments, preferably VHHs or fragments thereof may be linked to each other or may be interconnected. In particular embodiments, two or more antibody fragments, preferably two or more VHHs or fragments thereof are linked to each other via one or more suitable linkers or spacers. Suitable spacers or linkers for use in the coupling of such antibody fragment, as disclosed herein will be clear to the skilled person and may generally be any linker or spacer used in the art to link peptides and/or proteins.

Some particularly suitable linkers or spacers include for example, but are not limited to, polypeptide linkers such as glycine linkers, serine linkers, mixed glycine/serine linkers, glycine- and serine-rich linkers or linkers composed of largely polar polypeptide fragments, or homo- or heterobifunctional chemical crosslinking compounds such as glutaraldehyde or, optionally PEG-spaced, maleimides or NHS esters.

For example, a polypeptide linker or spacer may be a suitable amino acid sequence having a length between 1 and 50 amino acids, such as between 1 and 30, and in particular between 1 and 10 amino acid residues. It should be clear that the length, the degree of flexibility and/or other properties of the linker(s) may have some influence on the properties of the antibody fragments, preferably VHHs or fragments thereof, including but not limited to the affinity, specificity or avidity for the tumor target or the target on a cancer cell or pharmacological behavior. It should be clear that when two or more linkers are used, these linkers may be the same or different. In the context and disclosure of the present invention, the person skilled in the art will be able to determine the optimal linkers for the purpose of coupling antibody fragments, preferably VHHs or fragments thereof as disclosed herein without any undue experimental burden.

As used herein, the term ‘untagged’ when referring to an antibody fragment, such as a VHH or functional fragments thereof, denotes an antibody fragment that contains no extraneous polypeptide sequences (e.g., contains only an antibody fragment, preferably a VHH sequence, or a fragment thereof, preferably linked to a medicament and/or labeled with a radioisotope as described herein). Exemplary extraneous polypeptide sequences include carboxy-terminal polypeptide tags, e.g., a His-tag, a cysteine-containing tag (e.g., a GGC-tag as described in Pruszynski et al 2013 Nucl Med Biol 40: 52-59), and/or a Myc-tag. A His-tag may contain 4, 5, 6, 7, 8, 9, 10 Histidines. In an embodiment, 6 Histidines are present.

Also in one embodiment, the one or more groups, residues or moieties that may be present do not induce multimerization such as dimerization of the antibody fragment, preferably VHH or functional fragments thereof as disclosed herein.

Therefore in an embodiment, an antibody fragment such as a VHH or a fragment thereof is devoid of a tag that induces multimerization such as dimerization, preferably devoid of a cysteine-containing tag, preferably a GGC-tag.

Therefore in an embodiment, an antibody fragment such as a VHH or a fragment thereof is devoid of a carboxy-terminal polypeptide tag, preferably it is untagged.

Advantageously, kidney retention was shown to be significantly reduced when using an antibody fragment without a carboxy-terminal polypeptide tag compared to a polypeptide tagged, such as His-tagged and Myc-His-tagged antibody fragment (D'Huyvetter et al. (2014), Theranostics. 4(7):708-20).

The term ‘bi-specific’ when referring to an antibody fragment, such as a VHH, as disclosed herein implies that either a) two or more of the binding sites of an antibody fragment as disclosed herein are directed against or specifically bind human and/or murine FAP but not to the same (i.e. to a different) site, determinant, part, epitope, domain or stretch of amino acid residues of human and/or murine FAP, the antibody fragment as disclosed herein is said to be ‘bi-specific’ (in the case of two binding sites on the antibody fragment or multispecific (in the case of more than two binding sites on the antibody fragment) or b) two or more binding sites of an antibody fragment as disclosed herein are directed against or specifically bind to different target molecules of interest. The term ‘multispecific’ is used in the case that more than two binding sites are present on the antibody fragment as disclosed herein.

Accordingly, a ‘bispecific’ antibody fragment, such as a ‘bispecific’ VHH or a ‘multi-specific’ antibody fragment, such as a ‘multispecific’ VHH as used herein, shall have the meaning of an antibody fragment, such as a VHH, as disclosed herein comprising respectively two or at least two binding sites, wherein these two or more binding sites have a different binding specificity. Thus, an antibody fragment, such as a VHH, as disclosed herein is considered ‘bispecific’ or ‘multispecific’ if respectively two or more than two different binding regions exist in the same, monomeric antibody fragment.

The ‘half-life’ of an antibody fragment, in particular such as a VHH or fragments thereof, as disclosed herein can generally be defined as the time that is needed for the in vivo serum concentration of the antibody fragment, as disclosed herein to be reduced by 50%. The in vivo half-life of an antibody fragment, as disclosed herein can be determined in any manner known to the person skilled in the art, such as by pharmacokinetic analysis. As will be clear to the skilled person, the half-life can be expressed using parameters such as the t1/2-alpha, t1/2-beta and the area under the curve (AUC). An increased half-life in vivo is generally characterized by an increase in one or more and preferably in all three of the parameters t1/2-alpha, t1/2-beta and the area under the curve (AUC).

The term “lifetime extended” when referring to an antibody fragment, such as a VHH or fragments thereof as disclosed herein, is used to denote that the antibody fragment has been modified to extend the half-life of the antibody fragment. Strategies for extending the half-life of antibodies and antibody fragments are well-known in the art and include for example, but without limitation, linkage (chemically or otherwise) to one or more groups or moieties that extend the half-life, such as polyethylene glycol (PEG) or bovine serum albumin (BSA) or human serum albumin (HSA), antibody Fc fragments, or antigen-binding antibody fragments targeting serum proteins such as serum albumin.

Therefore, in an embodiment, the antibody fragment such as a VHH or a functional fragment thereof is non-lifetime extended.

Nucleic Acid Molecule Encoding the Antibody Fragment

In a further aspect, the present invention provides nucleic acid molecules represented by nucleic acid sequences encoding the antibody fragment, preferably the VHH or suitable fragments thereof as defined herein.

In an embodiment, this nucleic acid molecule is represented by a nucleic acid sequence that comprises, consists of or essentially consists of a nucleic acid sequence having at least 80% identity with any of SEQ ID NO: 33-36. Preferably, the identity is at least 80, 81, 82, 83, 84, 85, 86, 87, 88, 89, 90, 91, 92, 93, 94, 95, 96, 97, 98, 99 or 100%. The identity is usually assessed over the full length of said SEQ ID NO. However, it is not excluded the identity is assessed over a portion of said SEQ ID NO as defined herein.

These nucleic acid sequences can also be in the form of a vector or a genetic construct or polynucleotide. The nucleic acid sequences as disclosed herein may be synthetic or semi-synthetic sequences, nucleotide sequences that have been isolated from a library (and in particular, an expression library), nucleotide sequences that have been prepared by PCR using overlapping primers, or nucleotide sequences that have been prepared using techniques for DNA synthesis known per se.

TABLE 4
nucleic acid molecule encoding the antibody fragment

| | | | Translates to |
|---|---|---|---|
| VHH | SEQ ID NO: | DNA sequence | SEQ ID NO: |
| B1 | 33 | GATGTGCAGCTGGTGGAGTCTGGGGGAGGCTTGGTGCA | 4 |
| | | GCCTGGGGGGTCTCTGAGACTCTCCTGTGTAGCCTCTG | |
| | | GAAGGCTCTCCAGTAGCAATTCCATGGCCTGGTATCGC | |
| | | CAGGTTCCAGGGAAGCGTCGCGAGTTGGTCGCGGGAAT | |
| | | TACTGGTGGTGGTGAGACAAACTATGCAGACTTCGTGG | |
| | | GTGGCCGATTCACCATCTCCAGAGACAACGCCAAGAAC | |
| | | GGGCTGTATCTGCAATTGAACGGCCTGAAACCTGAGGA | |
| | | CACGGCCGCCTATTATTGTAATTTCTGGCCCCCACTTAT | |
| | | CAACTACTGGGGCCAAGGGACCCAGGTCACCGTCTCCT | |
| | | CA | |
| B2 | 34 | GATGTGCAGCTGGTGGAGTCTGGGGGAGGCTTGGTGCA | 8 |
| | | GGCTGGGGGGTCTCTGAGACTCTCCTGTGCAGTTTCTG | |
| | | GAAGCATCTCCAGTGCCAATAGCATGGGCTGGTACCGC | |
| | | CAGGCTCCAGGGAAGCAGCGCGACGTGGTCGCAGGTC | |
| | | TTACTACTGGTGGTAGGAGTCACTATGCAGACTCCGTGA | |
| | | AGGGCCGATTCACCATCTCCAGAGACAATGCCAAGAAC | |
| | | ACGGTGTATCTGCAAATGAACAGCCTGAAAGCAGAGGA | |
| | | CACGGCCGTCTATTACTGTAATTTGTGGCCGCCGGTTCA | |
| | | GGGCTACTGGGGCCAGGGGACCCAGGTCACCGTCTCC | |
| | | TCA | |
| B3 | 35 | GATGTGCAGCTGGTGGAGTCTGGGGGAGGCTTGGTGCA | 12 |
| | | GGCTGGGGGGTCTCTGAGACTCTCCTGTGCAGTCTCTG | |
| | | GAAGACTCTTCAGTACCAATGCCATGGGCTGGTACCGC | |
| | | CAGGCTCCAGGGAAGCAGCGCGAGTTGGTCGCAGGCA | |
| | | TTACTGGTGGTGATAGATCAAACTATGCAGACTCCGTGA | |
| | | AGGGCCGATTCACCATCTCCAGAGACAATGGCAAGAAC | |
| | | ACGCTGTATCTGCAAATGAACAGCCTGAAACCTGAGGAC | |
| | | ACGGCCGTCTATTACTGTAATTTCTACCCGCCTATTGTG | |
| | | GGTGACTACTGGGGCCAGGGGACCCAGGTCACCGTCTC | |
| | | CTCA | |
| B4 | 36 | GATGTGCAGCTGGTGGAGTCTGGGGGAGGATTGGTGCA | 16 |
| | | GGTTGGGGGCTCTCTGAGACTCTCCTGTGTAGCCTCTG | |
| | | GATTCACCTTCAGTAGCTACTACATGAGCTGGGTCCGCC | |
| | | AGGCTCCAGGGAAGGGGCTGGAGTGGGTGGCCAGTAT | |
| | | TTATGCTGACGGTGATATGACATACTATGCAGACTCCGT | |
| | | GAGGGGCCGATTCACCATCTCCAGAGACAACGCCAAGA | |
| | | ACACGCTGTATCTGCAAATGAACAGTCTGAAATCTGAGG | |
| | | ACACGGCCGTGTATTACTGTGCAAAAGATCCCCTCCCCC | |
| | | CCTATCATGTTAACCAGGGGACCCAGGTCACCGTCTCCT | |
| | | CA | |

Constructs, Vectors, Host Cells

The genetic constructs as disclosed herein may be DNA or RNA, and are preferably double-stranded DNA. The genetic constructs of the invention may also be in a form suitable for transformation of the intended host cell or host organism in a form suitable for integration into the genomic DNA of the intended host cell or in a form suitable for independent replication, maintenance and/or inheritance in the intended host organism. For instance, the genetic constructs of the invention may be in the form of a vector, such as for example a plasmid, cosmid, YAC, a viral vector or transposon. In particular, the vector may be an expression vector, i.e., a vector that can provide for expression in vitro and/or in vivo (e.g. in a suitable host cell, host organism and/or expression system).

Accordingly, in another further aspect, the present invention also provides vectors comprising one or more nucleic acid sequences as disclosed herein.

In still a further aspect, the present invention provides hosts or host cells or cells that comprise and preferably express or are capable of expressing one or more nucleic acid sequences and therefore one or more amino acid sequences as disclosed herein. Suitable examples of hosts or host cells will be clear to the skilled person.

Methods of Producing and Manufacturing Antibody Fragments

The invention further provides methods for preparing or generating the antibody fragment, in particular such as a VHH or fragments thereof, as well as methods for producing nucleic acids encoding these and host cells, products and compositions comprising these antibody fragments, in particular such as a VHH or fragments thereof. Some preferred but non-limiting examples of such methods will become clear from the further description herein.

As will be clear to the skilled person, one particularly useful method for preparing an antibody fragment, in particular such as a VHH or fragments thereof as disclosed herein generally comprises the steps of:
(a) expressing a nucleotide sequence encoding an antibody fragment, in particular such as a VHH or fragments thereof as disclosed herein and
(b) optionally isolating and/or purifying said antibody fragment.

The nucleic acid encoding the antibody fragment may be comprised in a vector or genetic construct.

In particular embodiments envisaged herein, the antibody fragment, in particular such as a VHH or fragments thereof can be obtained by methods which involve generating a random library of VHH sequences and screening this library for a VHH sequence capable of specifically binding to human and/or murine FAP.

Accordingly, in particular embodiments, methods for preparing an antibody fragment, in particular such as a VHH or fragments thereof as disclosed herein comprise the steps of
a) providing a set, collection or library of amino acid sequences of VHH domains; and
b) screening said set, collection or library of amino acid sequences for amino acid sequences that can bind to and/or have affinity for human and/or murine FAP.
and
c) isolating the amino acid sequence(s) that can bind to and/or have affinity for human and/or murine FAP.

In such a method, the set, collection or library of VHH sequences may be any suitable set, collection or library of amino acid sequences. For example, the set, collection or library of amino acid sequences may be a set, collection or library of immunoglobulin fragment sequences (as described herein), such as a naïve set, collection or library of immunoglobulin fragment sequences; a synthetic or semi-synthetic set, collection or library of immunoglobulin fragment sequences; and/or a set, collection or library of immunoglobulin fragment sequences that have been subjected to affinity maturation.

In particular embodiments of this method, the set, collection or library of VHH sequences may be an immune set, collection or library of immunoglobulin fragment sequences, for example derived from a mammal that has been suitably immunized with human and/or murine FAP or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of VHH sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art, for example on the basis of the further disclosure herein. Reference is also made to the review by Hoogenboom in Nature Biotechnology, 23, 9, 1105-1116 (2005).

In other embodiments, the methods for generating the antibody fragment, in particular such as a VHH or fragments thereof as disclosed herein comprise at least the steps of:
a) providing a collection or sample of cells expressing VHH domain amino acid sequences;
b) screening said collection or sample of cells for cells that express an amino acid sequence that can bind to and/or have affinity for human and/or murine FAP;
and
c) either (i) isolating said amino acid sequence; or (ii) isolating from said cell a nucleic acid sequence that encodes said amino acid sequence, followed by expressing said amino acid sequence.

The collection or sample of cells may for example be a collection or sample of B-cells. Also, in this method, the sample of cells may be derived from a mammal that has been suitably immunized with human and/or murine FAP or with a suitable antigenic determinant based thereon or derived therefrom, such as an antigenic part, fragment, region, domain, loop or other epitope thereof. In one particular embodiment, the antigenic determinant may be an extracellular part, region, domain, loop or other extracellular epitope(s).

In other embodiments, the method for generating an antibody fragment, in particular such as a VHH or fragments thereof as disclosed herein directed against human and/or murine FAP may comprise at least the steps of:
a) providing a set, collection or library of nucleic acid sequences encoding a VHH domain amino acid sequence;
b) screening said set, collection or library of nucleic acid sequences for nucleic acid sequences that encode an amino acid sequence that can bind to and/or has affinity for human and/or murine FAP;
and
c) isolating said nucleic acid sequence, followed by expressing said amino acid sequence.

In the above methods, the set, collection or library of nucleic acid sequences encoding amino acid sequences may for example be a set, collection or library of nucleic acid sequences encoding a naïve set, collection or library of immunoglobulin fragment sequences; a set, collection or library of nucleic acid sequences encoding a synthetic or semi-synthetic set, collection or library of immunoglobulin fragment sequences; and/or a set, collection or library of nucleic acid sequences encoding a set, collection or library of immunoglobulin fragment sequences that have been subjected to affinity maturation.

In particular, in such a method, the set, collection or library of nucleic acid sequences encodes a set, collection or library of an antibody fragment, in particular such as a VHH or fragments thereof as disclosed herein directed against human and/or murine FAP (as defined herein).

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example on the basis of the further disclosure herein. Reference is also made to the review by Hoogenboom in Nature Biotechnology, 23, 9, 1105-1116 (2005).

The invention also relates to an antibody fragment, in particular such as a VHH or fragments thereof as disclosed herein that are obtainable or obtained by the above methods, or alternatively by a method that comprises one of the above methods and in addition at least the steps of determining the nucleotide sequence or amino acid sequence of said antibody fragment, in particular such as a VHH or fragments thereof as disclosed herein; and of expressing or synthesizing said antibody fragment, in particular such as a VHH or fragments thereof as disclosed herein in a manner known per se, such as by expression in a suitable host cell or host organism or by chemical synthesis.

In some cases, the methods for producing the antibody fragment, in particular such as a VHH or fragments thereof as disclosed herein binding specifically to human and/or murine FAP as envisaged herein may further comprise the step of isolating from the amino acid sequence library at least one antibody fragment, in particular such as a VHH or fragments thereof as disclosed herein having detectable binding affinity for, or detectable in vitro effect on human and/or murine FAP.

These methods may further comprise the step of amplifying a sequence encoding at least one antibody fragment, in particular such as a VHH or fragments thereof as disclosed herein having detectable binding affinity for, or detectable in vitro effect on the activity of human and/or murine FAP. For example, a phage clone displaying a particular amino acid sequence, obtained from a selection step of a method described herein, may be amplified by reinfection of a host bacteria and incubation in a growth medium.

In particular embodiments, these methods may encompass determining the sequence of the one or more amino acid sequences capable of binding to human and/or murine FAP.

Where an antibody fragment, in particular such as a VHH or fragments thereof as disclosed herein, comprised in a set, collection or library of amino acid sequences, is displayed on a suitable cell or phage or particle, it is possible to isolate from said cell or phage or particle, the nucleotide sequence that encodes that amino acid sequence. In this way, the nucleotide sequence of the selected amino acid sequence library member(s) can be determined by a routine sequencing method.

In further particular embodiments, the methods for producing an antibody fragment, in particular such as a VHH or fragments thereof as envisaged herein comprise the step of expressing said nucleotide sequence(s) in a host organism under suitable conditions, so as to obtain the actual desired amino acid sequence. This step can be performed by methods known to the person skilled in the art.

In addition, the obtained antibody fragment, in particular such as a VHH or fragments thereof as disclosed herein having detectable binding affinity for, and/or no detectable in vitro effect on an activity of human and/or murine FAP, may be synthesized as soluble protein construct, optionally after their sequence has been identified.

For instance, the antibody fragment, in particular such as a VHH or fragments thereof as disclosed herein obtained, obtainable or selected by the above methods can be synthesized using recombinant or chemical synthesis methods known in the art. Also, the amino acid sequences obtained, obtainable or selected by the above methods can be produced by genetic engineering techniques. Thus, methods for synthesizing the antibody fragment, in particular such as a VHH or fragments thereof as disclosed herein obtained, obtainable or selected by the above methods may comprise transforming or infecting a host cell with a nucleic acid or a vector encoding an amino acid sequence having detectable binding affinity for, and/or no detectable in vitro effect on an activity of human and/or murine FAP. Accordingly, the antibody fragment, in particular such as a VHH or fragments thereof as disclosed herein having detectable binding affinity for, and/or no detectable in vitro effect on an activity of human and/or murine FAP can be made by recombinant DNA methods. DNA encoding the amino acid sequences can be readily synthesized using conventional procedures. Once prepared, the DNA can be introduced into expression vectors, which can then be transformed or transfected into host cells such as E. coli or any suitable expression system, in order to obtain the expression of amino acid sequences in the recombinant host cells and/or in the medium in which these recombinant host cells reside.

It should be understood, as known by someone skilled in the art of protein expression and purification, that the antibody fragment, in particular such as a VHH or fragments thereof as disclosed herein produced from an expression vector using a suitable expression system may be tagged (typically at the N-terminal or C-terminal end of the amino acid sequence) with e.g. a His-tag or other sequence tag for easy purification.

Transformation or transfection of nucleic acids or vectors into host cells may be accomplished by a variety of means known to the person skilled in the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

Suitable host cells for the expression of the desired heavy chain variable domain sequences may be any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic plant.

Thus, the application also provides methods for the production of an antibody fragment, in particular such as a VHH or fragments thereof as disclosed herein having detectable binding affinity for, or detectable in vitro effect on the activity of human and/or murine FAP comprising transforming, transfecting or infecting a host cell with nucleic acid sequences or vectors encoding such antibody fragment, in particular such as a VHH or fragments thereof as disclosed herein and expressing their amino acid sequences under suitable conditions.

In yet another embodiment, the invention further provides methods for the manufacture (‘or the production of’ which is equivalent wording) a pharmaceutical composition as disclosed herein.

In particular embodiments, the invention provides methods for producing a pharmaceutical composition as disclosed herein, at least comprising the steps of:
obtaining at least one antibody fragment, in particular such as a VHH or fragments thereof as disclosed herein, which specifically binds to human and/or murine FAP, and
formulating said antibody fragment, in particular such as a VHH or fragments thereof in a pharmaceutical composition.

In particular embodiments of these methods, the step of obtaining at least one antibody fragment, in particular such as a VHH or fragments thereof as disclosed herein, which specifically binds to human and/or murine FAP comprises:
(a) expressing a nucleotide sequence encoding an antibody fragment, in particular such as a VHH or fragments thereof as disclosed herein, which specifically binds to human and/or murine FAP, and optionally
(b) isolating and/or purifying the antibody fragment, in particular such as a VHH or fragments thereof as disclosed herein.

In other particular embodiments of these methods, the step of obtaining at least one antibody fragment, in particular such as a VHH or fragments thereof as disclosed herein, which specifically binds to human and/or murine FAP comprises:
a) providing a set, collection or library of VHH domain sequences or fragments of VHH sequences;
b) screening said set, collection or library of VHH domain sequences or sequences of fragments thereof for sequences that specifically bind to and/or have affinity for human and/or murine FAP, and optionally
c) isolating the VHH sequences or sequences of fragments thereof that specifically bind to and/or have affinity for human and/or murine FAP.

Process for Labeling the Antibody Fragment

There are various radiolabeling strategies available to incorporate a radionuclide into a protein. The choice of technique for a radiochemist depends primarily on the radionuclide used. The radioactive isotopes of iodine possess the ability to be directly integrated into a molecule by electrophilic substitution or indirectly via conjugation. Radioactive metals on the other hand are labeled via complexation with a chelating agent. Many metallic radionuclides possess the ability to form stable complexes with chelating agents, thus allowing for conjugation with a protein. Radiolabeling molecules with iodine nuclides is of great importance in pharmaceutical radiochemistry. There are over thirty different identified iodine isotopes, but only four are commonly used in radioiodine chemistry: ¹²³I, ¹²⁴I, ¹²⁵I and ¹³¹I.

The direct radioiodination of a protein is a key method for the synthesis of tumor-targeting or cancer cell-targeting radiopharmaceuticals. Generally, there are two basic approaches of protein radioiodination. The most straightforward approach is direct protein labeling using electrophilic substitution at tyrosine and histidine residues. The radioiodide is oxidized in situ creating the electrophile *I⁺. This is done using oxidizing agents like chloramine T, Iodogen® and N-halosuccinimides. The generated electrophile attacks the electron-rich aromatic ring of the amino acid tyrosine, forming a a-complex. This substitution is performed at the tyrosine residue due to the electron donating hydroxyl group which stabilizes the a-complex. As the labeling of proteins must take place under mild conditions, the attachment of iodine to the tyrosine is highly suitable.

This method is performed under mild conditions, which is optimal for the labeling of proteins. This is however only possible when the protein contains accessible tyrosine or histidine residues.

Indirect iodination of proteins via conjugation is a frequently used alternative method. In this approach iodine is incorporated by the application of prosthetic groups containing two functional groups to enable both radioiodination and incorporation to the protein. There are a variety of prosthetic groups used for radioiodination, but the most frequently used are N-succinimidyl-5-[*I]iodo-3-pyridinecarboxyl ([¹³¹I]SIPC) and N-succinimidyl-3-[*I]-iodobenzoate ([*I]SIB). Both active esters are conjugated to amino groups of the protein and exhibit a high in vivo stability.

Another prosthetic group for the acylation of aromatic groups is N-succinimidyl-4-guanidinomethyl-3-[I-131]iodobenzoate ([I-131]SGMIB).

In particular embodiments of the present invention, the labelled compounds as disclosed herein are labelled with Iodine-131 using N-succinimidyl-4-guanidinomethyl-3-[I-131]iodobenzoate ([I-131]SGMIB) or suitable derivatives or variants thereof.

Detailed protocols for radiotherapy are readily available to the expert (Cancer Radiotherapy: Methods and Protocols (Methods in Molecular Medicine), Huddart RA Ed., Human Press 2002). The skilled person knows how to determine an appropriate dosing and application schedule, depending on the nature of the disease and the constitution of the patient. In particular, the skilled person knows how to assess dose-limiting toxicity (DLT) and how to determine the maximum tolerated dose (MTD) accordingly.

In particular embodiments, the labelled compounds thereof as disclosed herein are administered at a radioactive dosage of lower than about 800 mCi, such as for instance lower than about 150 mCi, such as for instance lower than about 30 mCi, such as lower than about 15 mCi.

In particular embodiments, the radioimmunoconjugate has a specific activity from about 0.5 mCi/mg to about 8000 mCi/mg, such as for instance from 1 mCi/mg to about 1500 mCi/mg, such as for instance from 1 mCi/mg to about 300 mCi/mg, such as for instance from 1 mCi/mg to about 150 mCi/mg, depending on the radionuclide, and may be administered via an intravenous, intraperitoneal or other route such as intrathecal route. Depending on the desired duration and effectiveness of the treatment, the labelled compounds as disclosed herein may be administered once or several times, in combination with other therapeutic drugs or radio-sensitizing agents. The amount of the labelled compounds applied depends on the precise nature of the carcinoma. The dose of radioactivity per administration must be high enough to be effective, but must be below the dose limiting toxicity (DLT).

Formulation/Uses in Therapy/Diagnostic

In yet a further aspect, compositions are provided comprising one or more antibody fragment, preferably VHH or fragments thereof disclosed herein and/or nucleic acid sequences as envisaged herein and optionally at least one acceptable carrier.

According to certain particular embodiments, the compositions as envisaged herein may further optionally comprise at least one other compound.

As used herein, a “screening dose” or a “biomarker dose” is a dose of an agent, such as a labelled compound as described herein, that is sufficient for selecting a subject for treatment, such as a dose that can bind to a cancer cell or solid tumor in the subject and subsequently be detected at the location of the cancer cell or solid tumor, e.g., by imaging the subject using gamma camera imaging such as planar gamma camera imaging, single photon emission computed tomography or positron emission tomography, optionally combined with a non-nuclear imaging technique such as X-ray imaging, computed tomography and/or magnetic resonance imaging. In some embodiments, a screening dose is a dose that is not therapeutically effective. In some embodiments, the screening dose is different than (e.g., lower than) a therapeutic dose as described herein.

As used herein, a “therapeutic dose” is a dose of an agent, such as a labelled compound as described herein, that is therapeutically effective in at least 1%, at least 5%, at least 10%, at least 20%, at least 30%, at least 40%, or at least 50% of subjects in need of such treatment (e.g., in subjects having cancer). In some embodiments, the therapeutic dose is higher than a screening dose as described herein.

As used herein, “imaging a subject” refers to capturing one or more images of a subject using a device that is capable of detecting a labelled compound as described herein. The one or more images may be further altered by a computer program and/or a person skilled in the art in order to enhance the images (e.g. by adjusting contrast or brightness of the one or more images). Any device capable of detecting a labelled compound as described herein is contemplated for use, such as a device for gamma camera imaging such as planar gamma camera imaging, for single photon emission computed tomography or for positron emission tomography, or a device able to combine a nuclear imaging technique with an anatomical imaging technique such as X-ray imaging, computed tomography and/or magnetic resonance imaging. For example, such device can be a device for single photon emission computed tomography/computed tomography (SPECT/CT) or positron emission computed tomography/computed tomography (PET/CT) imaging. Such devices are known in the art and commercially available.

In some embodiments, the administration of the screening dose and the detection by imaging are separated by at least 1 about minute, at least 5 about minutes, at least about 10 minutes, at least about 20 minutes, at least about 30 minutes, at least about 40 minutes, at least about 50 minutes, at least about 1 hour, at least about 1.5 hours, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 24 hours, at least about 2 days, or at least about 7 days. In some embodiments, the administration of the screening dose and the detection are separated by between about 1 hour and about 24 hours.

In some embodiments, the screening dose and the therapeutic dose are administered at least about 1 day, at least about 2 days, at least about 3 days, at least about 4 days, at least about 5 days, at least about 6 days, at least about 1 week, at least about 2 weeks, at least about 3 weeks, at least one month, at least about 2 months, or at least about 6 months apart. In some embodiments, the screening dose and the therapeutic dose are administered between about 1 day and about 6 months apart (e.g., between about 1 day and about 2 months, between about 1 day and about 1 month, or between about 1 day and about 1 week apart).

The screening dose and therapeutic dose may each independently be administered by any suitable route, such as systemically, locally or topically. Exemplary routes include intravenous, intraperitoneal, and intrathecal administration. The particular route utilized may, in some embodiments, depend on the nature of the disease (e.g., type, grade, location and stage of the tumor or cancer cell etc.) and the type of subject (e.g., species, constitution, age, gender, weight, etc.).

As used herein for all diagnostic and therapeutic applications, the term “subject” generally refers to a mammal, such as a human, a non-human primate, a rat, a mouse, a rabbit, a dog, a cat, a pig, a horse, a goat, or a sheep. In some embodiments, the subject is a human subject. In some embodiments, the subject is a subject having cancer (e.g., a human subject having cancer). Methods for identifying subjects having cancer include detection of tumor antigens or other tumor biomarkers, genetic testing, MRI, X-ray, PET or SPECT scan, biopsies, and combinations thereof.

As used herein, the terms ‘diagnosis’, ‘prediction’ and/or ‘prognosis’ as used herein comprise diagnosing, predicting and/or prognosing a certain disease and/or disorder and/or condition, thereby predicting the onset and/or presence of a certain disease and/or disorder and/or condition, and/or predicting the progress and/or duration of a certain disease and/or disorder and/or condition, and/or predicting the response of a patient suffering from of a certain disease and/or disorder and/or condition to therapy.

In some embodiments of any one of the labelled compounds, composition comprising the same or diagnostic or therapeutic applications provided, a screening dose (i.e. used in a diagnostic method) is a dose that is not therapeutically effective. In some embodiments, the screening dose is lower than a therapeutic dose as described herein (e.g., at least about 10 times, at least about 20 times, at least about 30 times, at least about 40 times, at least about 50 times, at least about 100 times, at least about 200 times, at least about 300 times, at least about 400 times, at least about 500 times or at least about 1000 times lower than a therapeutic dose as described herein, or at least about 10, at least about 20, at least about 30, at least about 40, at least about 50, at least about 60, at least about 70, at least about 80, at least about 90, at least about 100, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 320, at least about 340, at least about 360, at least about 380, at least about 400, at least about 450, at least about 500, at least about 1000, at least about 5000, at least about 10000, at least about 13000, at least about 15000, at least about 18000, or at least about 20000 MBq lower than a therapeutic dose as described herein). In some embodiments, the screening dose is between 10 about MBq and about 400 MBq, between about 20 MBq and about 400 MBq, between about 30 MBq and about 400 MBq, about 40 MBq and about 400 MBq, between about 50 MBq and about 400 MBq, between about 100 MBq and about 400 MBq, between about 200 MBq and about 400 MBq, between about 300 MBq and about 400 MBq, between about 10 MBq and about 300 MBq, between about 20 MBq and about 300 MBq, between about 30 MBq and about 300 MBq, about 40 MBq and about 300 MBq, between about 50 MBq and about 300 MBq, between about 100 MBq and about 300 MBq, or between about 200 MBq and about 300 MBq. In some embodiments, the screening dose is between 3 about 7 MBq and about 370 MBq. It is to be understood that any screening dose described herein may be combined with any therapeutic dose as described herein.

In some embodiments, the therapeutic dose is higher than a screening dose as described herein (e.g., at least about 10 times, at least about 20 times, at least about 30 times, at least about 40 times, at least about 50 times, at least about 100 times, at least about 200 times, at least about 300 times, at least about 400 times, at least about 500 times or at least about 1000 times higher than a screening dose as described herein, or at least about 10, at least about 20, at least about 30, at least about 40, at least about 50, at least about 60, at least about 70, at least about 80, at least about 90, at least about 100, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 320, at least about 340, at least about 360, at least about 380, at least about 400, at least about 450, at least about 500, at least about 1000, at least about 5000, at least about 10000, at least about 13000, at least about 15000, at least about 18000, or at least about 20000 MBq higher than a screening dose as described herein). In some embodiments, the therapeutic dose is between about 300 MBq and about 20000 MBq, between about 400 MBq and about 20000 MBq, between about 500 MBq and about 20000 MBq, between about 1000 MBq and about 20000 MBq, between about 2000 MBq and about 20000 MBq, between about 3000 MBq and about 20000 MBq, between about 4000 MBq and about 20000 MBq, between about 5000 MBq and about 20000 MBq, between about 10000 MBq and about 20000 MBq, between about 5000 MBq and about 20000 MBq, between about 10000 MBq and about 20000 MBq, between about 300 MBq and about 10000 MBq, between about 400 MBq and about 10000 MBq, between about 500 MBq and about 10000 MBq, between about 1000 MBq and about 10000 MBq, between about 2000 MBq and about 10000 MBq, between about 3000 MBq and about 10000 MBq, between about 4000 MBq and about 10000 MBq, or between about 5000 MBq and about 10000 MBq. In some embodiments of any one of the methods provided, the therapeutic dose is between about 370 MBq and about 18500 MBq.

The screening and/or therapeutic dose may conveniently be presented in a single dose or as divided doses (which can again be sub-dosed) administered at appropriate intervals. An administration regimen of the therapeutic dose could include long-term (e.g., at least two weeks, and for example several months or years) or daily treatment. In some embodiments, an administration regimen of the therapeutic dose can vary between once a day to once a month, such as between once a day and once every two weeks, such as but not limited to once a week. Thus, in some embodiments, pharmaceutical compositions as disclosed herein may be administered once or several times, also intermittently, for instance on a daily basis for several days, weeks or months.

The particular screening dose and therapeutic dose utilized may, in some embodiments, depend on the nature of the disease (such as cancer but also the type, grade, and stage of the tumor or cancer cell etc., or such as any of the other diseases identified herein) and the type of subject (e.g., species, constitution, age, gender, weight, etc.).

In some aspects, the invention provides kits, such as a kit for diagnostic and therapeutic applications as described herein. In some embodiments, the kit comprises a screening dose of a labelled compound as described herein and a therapeutic dose of the same compound. Screening doses and therapeutic doses are described herein.

In some embodiments of any one of the kits, the kit further comprises one or more means for injection of the screening dose and the therapeutic dose. In some embodiments, the screening dose and therapeutic dose are each individually housed in a means for injection. In some embodiments, the means for injection is a syringe. In some embodiments of any one of the kits, the kit further comprises instructions for carrying out a method as described herein (e.g., a method of stratifying and treating a subject as described herein). The instructions may be in any suitable form, e.g., in printed form (e.g., as a paper or laminated insert or label) or in electronic form (e.g., on a disc or USB stick).

Dose, route of administration, application scheme, repetition and duration of treatment will in general depend on the nature of the disease (type, grade, and stage of the tumor or cancer cell or type, grade and stage of the disease or condition further defined herein) and the patient (constitution, age, gender etc.), and will be determined by the skilled medical expert responsible for the treatment. With respect to the possible doses for the components of the disclosed combination which are described above, it is clear that the medical expert responsible for the treatment will carefully monitor whether any dose-limiting toxicity or other severe side effects occur and undertake the necessary steps to manage those.

Generally, for pharmaceutical (diagnostic and therapeutic) use, the (labelled) compound comprising an antibody fragment, preferably VHH or fragments thereof as envisaged herein may be formulated as a pharmaceutical preparation or compositions comprising the (labelled) compound as envisaged herein and at least one pharmaceutically acceptable carrier, diluent or excipient and/or adjuvant, and optionally one or more further pharmaceutically active polypeptides and/or compounds. Such a labelled compound or composition comprising the same may be suitable for intraperitoneal, intravenous or other administration such as intrathecal administration. Thus, the (labelled) compounds and/or the compositions comprising the same can for example be administered systemically, locally or topically to the tissue or organ of interest, depending on the location, type and origin of the tumor or cancer cell, and preferably intraperitoneally, intravenously or intrathecally, depending on the specific pharmaceutical formulation or composition to be used. The clinician will be able to select a suitable route of administration and a suitable pharmaceutical formulation or composition to be used in such administration. The same holds for the compound which is to deliver a medicament to a cell, a tissue or an organ expressing or over-expressing FAP.

The pharmaceutical dosage forms suitable for injection or infusion can include sterile aqueous solutions or dispersions or sterile powders comprising the active ingredients which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof.

The amount of the (labelled) compound as envisaged herein required for use in prophylaxis and/or treatment may vary not only with the particular antibody fragment, preferably a VHH or functional fragments thereof but also with the route of administration, the nature of the condition being treated and the age and condition of the patient and will be ultimately at the discretion of the attendant physician or clinician. Also, the dosage of the (labelled) compound envisaged herein may vary depending on the target cell, tumor, tissue, graft, or organ.

In particular, the (labelled) compound as envisaged herein will be administered in an amount which will be determined by the medical practitioner based inter alia on the severity of the condition and the patient to be treated. Typically, for each disease indication an optimal dosage will be determined specifying the amount to be administered per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment.

Useful dosages of the (labelled) compound thereof as envisaged herein can be determined by determining their in vitro activity, and/or in vivo activity in animal models. The non-human animal of the invention may be used to this end (see example 2).

In certain embodiments, the present invention provides a labelled compound as disclosed herein for use in the prevention and/or treatment of cancer (preferably cancer which is associated with the expression of human FAP on cancer cells and/or on CAF) by administering to a subject in need thereof the labelled compound at a dose ranging from 10 μg and 10 mg or from 10 μg and 7 mg or from 10 μg and 5 mg or from 10 μg and 2 mg or from 10 μg and 1.5 mg or from 10 μg and 1 mg of VHH. In further particular embodiments, the present invention provides a labelled compound as disclosed herein for use in the prevention and/or treatment of cancer by administering to a subject in need thereof the labelled compound at a dose ranging from 10 μg and 2 mg of labelled compound, such as in particular ranging from 10 μg and 1.5 mg or ranging from 100 μg and 1 mg of labelled compound.

Accordingly, the dose of radioactivity applied to the patient per administration has to be high enough to be effective but must be below the dose limiting toxicity (DLT). For pharmaceutical compositions comprising radiolabeled antibodies, e.g. with 131-Iodine, the maximally tolerated dose (MTD) has to be determined which must not be exceeded in therapeutic settings.

The compound and labeled compound as envisaged herein and/or the compositions comprising the same are administered according to a regimen of treatment that is suitable for preventing and/or treating the disease or disorder to be prevented or treated. The clinician will generally be able to determine a suitable treatment regimen. Generally, the treatment regimen will comprise the administration of a labelled compound, or of one or more compositions comprising the same, in one or more pharmaceutically effective amounts or doses.

The desired dose may conveniently be presented in a single dose or as divided doses (which can again be sub-dosed) administered at appropriate intervals. An administration regimen could include long-term (i.e., at least two weeks, and for example several months or years) or daily treatment. In particular, an administration regimen can vary between once a day to once a month, such as between once a day and once every two weeks, such as but not limited to once a week. Thus, depending on the desired duration and effectiveness of the treatment, labelled compound or composition comprising the same as disclosed herein may be administered once or several times, also intermittently, for instance on a daily basis for several days, weeks or months and in different dosages. The amount applied of the labelled compound or composition disclosed herein depends on the nature of the particular cancer disease. Multiple administrations are preferred. However, radiolabelled materials are typically administered at intervals of 1 to 20 weeks apart or 2 to 10 weeks apart or 2 to 8 weeks apart or 3 to 6 weeks apart or 3 to 5 weeks apart or each 4 weeks. The skilled artisan knows however how to choose dividing the administration into two or more applications, which may be applied shortly after each other, or at some other predetermined interval ranging e.g. from 1 day to 4 weeks.

In particular, the labelled compounds as envisaged herein may be used in combination with other pharmaceutically active compounds or principles that are or can be used for the prevention and/or treatment of the diseases and disorders cited herein, as a result of which a synergistic effect may or may not be obtained. Examples of such compounds and principles, as well as routes, methods and pharmaceutical formulations or compositions for administering them will be clear to the clinician.

In the context of this invention, “in combination with”, “in combination therapy” or “in combination treatment” shall mean that the labelled compound as disclosed herein or composition comprising these labelled compounds as disclosed herein are applied together with one or more other pharmaceutically active compounds or principles to the patient in a regimen wherein the patient may profit from the beneficial effect of such a combination. In particular, both treatments are applied to the patient in temporal proximity. In a preferred embodiment, both treatments are applied to the patient within four weeks (28 days). More preferably, both treatments are applied within two weeks (14 days), more preferred within one week (7 days). In a preferred embodiment, the two treatments are applied within two or three days. In another preferred embodiment, the two treatments are applied at the same day, i.e. within 24 hours. In another embodiment, the two treatments are applied within four hours, or two hours, or within one hour. In another embodiment, the two treatments are applied in parallel, i.e. at the same time, or the two administrations are overlapping in time.

In particular non-limiting embodiments, the labelled compounds or composition comprising these labelled compounds as disclosed herein are applied together with a molecule or a composition comprising it, wherein said molecule or composition comprising it is able to optimize and therefore reduce kidney retention of the labelled compound. In the context of the invention, “applied together with” is to be construed broadly. It means it encompasses applied simultaneously on one or in two distinct compositions. It also encompasses applied sequentially in two distinct compositions.

Such a molecule may be a plasma or blood substitute such as modified gelatin. An example of modified gelatin that may be used in this context is Gelofusine™. The use of such plasma or blood substitute is expected to optimize and therefore reduce the retention of the labeled compound in the kidney and therefore to optimize unwanted side effects. The advantage of using such a plasma or blood substitute with the compound of the invention has been demonstrated in examples 8 and 9.

Another example of such a molecule may be a positively charged amino acid or a composition comprising at least one positively charged amino acid. Examples of suitable positively charged amino acids are arginine, lysine and/or histidine. An example of such a composition is Aminomedix™. The use of positively charged amino acids has been extensively described in WO 2014/204854 which is explicitly incorporated by reference.

In particular non-limiting embodiments, the labelled compounds or composition comprising these labelled compounds as disclosed herein are applied together with immunotherapy. In an embodiment, with one or more therapeutic antibodies or therapeutic antibody fragments. Thus, in these particular non-limiting embodiments, the radioimmunotherapy with the labelled compounds as disclosed herein or composition comprising these labeled compounds is combined with regular immunotherapy with one or more therapeutic antibodies or therapeutic antibody fragments. In further particular embodiments, the labelled compounds as disclosed herein or composition comprising these labeled compounds as disclosed herein are used in a combination therapy or a combination treatment method with one or more therapeutic antibodies or therapeutic antibody fragments.

In an embodiment, there is provided a combination therapy comprising a labelled compound as defined herein and an additional antibody or antibody fragment.

For example, the labelled compounds as disclosed herein or composition comprising these labeled compounds and the one or more therapeutic antibodies or therapeutic antibody fragments may be infused at the same time, or the infusions may be overlapping in time. If the two drugs are administered at the same time, they may be formulated together in one single pharmaceutical preparation, or they may be mixed together immediately before administration from two different pharmaceutical preparations, for example by dissolving or diluting into one single infusion solution. In another embodiment, the two drugs are administered separately, i.e. as two independent pharmaceutical compositions. In one preferred embodiment, administration of the two treatments is in a way that tumour cells within the body of the patient are exposed to effective amounts of the cytotoxic drug and the radiation at the same time. In another preferred embodiment, effective amounts of both the labelled compounds as disclosed herein or composition comprising these labeled compounds as disclosed herein and the one or more therapeutic antibodies or therapeutic antibody fragments are present at the site of the tumour at the same time. The present invention also embraces the use of further agents, which are administered in addition to the combination as defined. This could be, for example, one or more further chemotherapeutic agent(s). It could also be one or more agent(s) applied to prevent, suppress, or ameliorate unwanted side effects of any of the other drugs given. For example, a cytokine stimulating proliferation of leukocytes may be applied to ameliorate the effects of leukopenia or neutropenia.

The efficacy of the compound or labelled compounds described herein, and of compositions comprising the same, can be tested using any suitable in vitro assay, cell-based assay, in vivo assay and/or animal model known per se, or any combination thereof, depending on the specific disease or disorder involved. Suitable assays and animal models will be clear to the skilled person. A suitable animal model is the non-human animal disclosed in example 2 as an aspect of the invention and expressing human FAP. The skilled person will generally be able to select a suitable in vitro or in vivo assay, cellular assay or animal model to test the antibody fragment, preferably VHH or fragments thereof or compound or labelled compound or composition comprising the same (all defined herein) for binding to human and/or murine FAP, as well as for their therapeutic and/or prophylactic effect in respect of one or more cancer-related diseases and fibrotic disorders. Such assay may be an imaging assay as disclosed herein.

The term ‘effective amount’, as used herein, means the amount needed to achieve the desired result or results.

As used herein, the terms ‘determining’, ‘measuring’, ‘assessing’, ‘monitoring’ and ‘assaying’ are used interchangeably and include both quantitative and qualitative determinations.

As used herein, the term ‘prevention and/or treatment’ comprises preventing and/or treating a certain disease and/or disorder and/or condition, preventing the onset of a certain disease and/or disorder and/or condition, slowing down or reversing the progress of a certain disease and/or disorder and/or condition, preventing or slowing down the onset of one or more symptoms associated with a certain disease and/or disorder and/or condition, reducing and/or alleviating one or more symptoms associated with a certain disease and/or disorder and/or condition, reducing the severity and/or the duration of a certain disease and/or disorder and/or condition, and generally any prophylactic or therapeutic effect of the antibody fragment as disclosed herein that is beneficial to the subject or patient being treated.

Cancer/Tumour/Metastatic Cell

As used herein, the term ‘tumor cell’ refers to a cell that is present in a primary or metastatic tumour lesion. In this context, tumours consist not only of cancer cells, but should be considered as organ-like structures in which a complex bidirectional interplay exists between transformed and non-transformed cells. The malignant potential of transformed cells requires an apt support structure from the stroma, which can consist of fibroblasts, adipocytes, blood and lymph vessels, but may also be considerably infiltrated by a wide range of immune cells. Within the context of the invention, a tumour cell may also be a fibroblast, preferably a CAF.

By “solid tumor(s)” or “tumor(s)” are meant primary tumors and/or metastases (wherever located).

As used herein, the term ‘cancer cell’ refers to a cell that divides and reproduces abnormally and limitlessly with uncontrolled growth and which can break away and travel to other parts of the body and set up another site, referred to as metastasis.

A ‘lesion’ as used herein can refer to any abnormal change in a body tissue or organ resulting from injury or disease. In cancer terminology, lesion typically refers to a tumour.

The term ‘primary tumour(s)’ as used herein is a tumor growing at the anatomical site where tumor progression began and proceeded to yield a cancerous mass.

The term ‘metastatic lesion(s)’ as used herein refers to malignant, or cancerous, tumours that have spread from their original location to other parts of the body. Related medical terms that might be used interchangeably include late-stage cancer, advanced cancer, or metastatic disease. In general, metastatic lesions are considered to be incurable, although treatment is often available to control the spread of cancerous cells and potentially increase the individual's life expectancy.

Metastasis is the term for the spread of cancer beyond its originating site in the body. Thus, metastatic lesions are cancerous tumours that are found in locations apart from the original starting point of the primary tumour. Metastatic tumours occur when cells from the primary tumour break off and travel to distant parts of the body via the lymph system and blood stream. Alternately, cells from the original tumour could seed into new tumours at adjacent organs or tissues. ‘Metastatic disease’ as used herein refers to late-stage cancer and to the medical classification of cancer as being in stage III, when cancer cells are found in lymph nodes near the original tumour, or in stage IV, when cancer cells have travelled far beyond the primary tumour site to distant parts of the body. Metastatic lesions are most commonly found in the brain, lungs, liver, or bones. An individual with metastatic cancer might or might not experience any symptoms, and the symptoms could be related to the area where metastasized cells have relocated. Once metastatic lesions are present in the body, the individual's cancer will be considered incurable for most cancer types. This means it is excessively difficult to eradicate every existing cancer cell with available treatments. In this case, the goal of treatment becomes slowing the growth of tumours to maintain the highest possible quality of life and potentially extend the individual's life expectancy. In some cases, people with metastatic lesions can live for a number of years with appropriate treatment for symptom management.

Radiolabel/Label/Radionuclide/Dose

As used herein, the term “labelled” as in “labelled compound” refers to the radioisotopic labeling of that antibody fragment or VHH or fragment thereof, wherein the antibody fragment or VHH or fragment thereof is labelled by including, coupling, or chemically linking a radionuclide to its amino acid sequence structure.

As used herein, the terms ‘radionuclide’, ‘radioactive nuclide’, ‘radioisotope’ or ‘radioactive isotope’, are used interchangeably herein and refer to atoms with an unstable nucleus, characterized by excess energy available to be imparted either to a newly created radiation particle within the nucleus or via internal conversion. During this process, the radionuclide is said to undergo radioactive decay, resulting in the emission of gamma ray(s) and/or subatomic particles such as alpha or beta particles. These emissions constitute ionizing radiation. Radionuclides occur naturally or can be produced artificially.

IHC Techniques

The term ‘immunohistochemistry (IHC)’ as used herein refers to the process of detecting antigens (e.g., proteins) in cells of a tissue section by exploiting the principle of antibodies binding specifically to antigens in sections of biological tissues. Immunohistochemical staining is widely used in the diagnosis of abnormal cells such as those found in cancerous tumors. IHC is also widely used in basic research to understand the distribution and localization of biomarkers and differentially expressed proteins in different parts of a biological tissue.

TABLE 5
Sequence listing

| SEQ | Amino acid/ | Characterization |
|---|---|---|
| ID NO | nucleic acid | of the sequence |
| 1 | Amino acid | CDR1 of B1 |
| 2 | Amino acid | CDR2 of B1 |
| 3 | Amino acid | CDR3 of B1 |
| 4 | Amino acid | B1 |
| 5 | Amino acid | CDR1 of B2 |
| 6 | Amino acid | CDR2 of B2 |
| 7 | Amino acid | CDR3 of B2 |
| 8 | Amino acid | B2 |
| 9 | Amino acid | CDR1 of B3 |
| 10 | Amino acid | CDR2 of B3 |
| 11 | Amino acid | CDR3 of B3 |
| 12 | Amino acid | B3 |
| 13 | Amino acid | CDR1 of B4 |
| 14 | Amino acid | CDR2 of B4 |
| 15 | Amino acid | CDR3 of B4 |
| 16 | Amino acid | B4 |
| 17 | Amino acid | B1 with HA-His6-tag |
| 18 | Amino acid | B2 with HA-His6-tag |
| 19 | Amino acid | B3 with HA-His6-tag |
| 20 | Amino acid | B4 with HA-His6-tag |
| 21 | Amino acid | B1 with His6-tag |
| 22 | Amino acid | B2 with His6-tag |
| 23 | Amino acid | B3 with His6-tag |
| 24 | Amino acid | B4 with His6-tag |
| 25 | Nucleic acid | nucleic acid encoding human |
| | | fibroblast activation protein |
| | | alpha (FAP), transcript variant 1 |
| 26 | Amino acid | human fibroblast activation protein |
| | | alpha (FAP), transcript variant 1 |
| 27 | Nucleic acid | targeting vector |
| 28 | Amino acid | human FAP recombinant protein |
| 29 | Amino acid | murine FAP recombinant protein |
| 30 | Amino acid | murine fibroblast activation |
| | | protein alpha (FAP) |
| 31 | Amino acid | human Cystatin-S signal peptide |
| 32 | Amino acid | nontargeting control VHH R3B23 |
| 33 | Nucleic acid | nucleic acid encoding B1 |
| 34 | Nucleic acid | nucleic acid encoding B2 |
| 35 | Nucleic acid | nucleic acid encoding B3 |
| 36 | Nucleic acid | nucleic acid encoding B4 |
| 37 | Amino acid | FR1 of B1 |
| 38 | Amino acid | FR2 of B1 |
| 39 | Amino acid | FR3 of B1 |
| 40 | Amino acid | FR4 of B1 |
| 41 | Amino acid | FR1 of B2 |
| 42 | Amino acid | FR2 of B2 |
| 43 | Amino acid | FR3 of B2 |
| 44 | Amino acid | FR4 of B2 |
| 45 | Amino acid | FR1 of B3 |
| 46 | Amino acid | FR2 of B3 |
| 47 | Amino acid | FR3 of B3 |
| 48 | Amino acid | FR4 of B3 |
| 49 | Amino acid | FR1 of B4 |
| 50 | Amino acid | FR2 of B4 |
| 51 | Amino acid | FR3 of B4 |
| 52 | Amino acid | FR4 of B4 |
| 53 | Amino acid | Exemplary HA tag |
| 54 | Amino acid | Exemplary HA tag |
| 55 | Amino acid | Exemplary cysteine tag |

All documents cited in the present specification are hereby incorporated by reference in their entirety. Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. Each embodiment described herein may be combined together with any other embodiment described herein, unless otherwise indicated.

The present invention is further described by the following examples which should not be construed as limiting the scope of the invention.

### EXAMPLES

### Example 1: Production of VHH B1, B2, B3 and B4, and Variants Thereof

The hemagglutinin- and hexahistidine-tagged (HA-His₆-tagged) variants of VHHs B1, B2, B3 and B4 (SEQ ID NO: 17-20) were produced in E. coli WK6 cells transduced with the VHH phagemid vector and extracted from the periplasm by either freeze-thaw or osmotic shock methods, according to standard protocols such as those in Vincke, C., et al. (2012). Methods in Molecular Biology 907: 145-176. In short, bacteria were grown in Terrific Broth medium and VHH expression was induced in the exponential growth phase. During overnight growth VHHs were translocated to the periplasm, from which they were extracted by a freeze-thaw cycle. Alternatively, the VHHs were collected from the periplasm by an osmotic shock, and purified to >80% purity via immobilized-metal affinity chromatography (IMAC) by batch incubation with HIS-select suspension (Sigma-Aldrich) and a stepwise elution with 0.5 M imidazole. The eluate was desalted via gel-filtration chromatography using Zeba Spin desalting columns (Thermo Fisher Scientific), equilibrated in PBS.

The hexahistidine-tagged (His₆-tagged) variants of VHHs B1, B2, B3 and B4 (SEQ ID NO: 21-24) were produced in E. coli WK6 cells transformed with a VHH recombinant expression plasmid and purified from the periplasm by IMAC and size-exclusion chromatography (SEC), according to standard protocols such as those in Vincke, C., et al. (2012). Methods in Molecular Biology 907: 145-176. In short, bacteria were grown in Terrific Broth medium and VHH expression was induced in the exponential growth phase. During overnight growth VHHs were translocated to the periplasm, from which they were collected after an osmotic shock. VHHs were purified from the periplasmic extract via IMAC by batch incubation with HIS-select suspension (Sigma-Aldrich) followed by a stepwise elution with 0.5 M imidazole. Subsequent SEC was performed on a HiLoad 16/600 Superdex 75 PG column (GE Healthcare) or Superdex 75 10/300 GL (GE Healthcare), equilibrated in PBS.

The untagged variant of VHH B1 (SEQ ID NO: 4) was produced in HEK293-E cells transiently transfected with endotoxin-free plasmid DNA according to standard protocols such as those in Baldi, et al. (2005) Biotechnol Prog. 21(1):148-53. Six days post transfection, conditioned medium containing the VHH was harvested by centrifugation. VHHs were purified from the harvested medium via cation-exchange chromatography using a Capto SP ImpRes column (GE Healthcare) equilibrated in 20 mM NaAc, pH 4.0 and employing a salt gradient. Eluate fractions were neutralized to pH 7.0 with 1 M Tris-HCl, pH 8.0. Subsequent SEC was performed on a Superdex 75 26/600 column (GE Healthcare), equilibrated in PBS.

TABLE 6
Variants of VHHs B1, B2, B3 and B4.

| Clone | Variant | SEQ ID NO | |
|---|---|---|---|
| B1 | HA-His6-tag | 17 | QVQLQESGGGLVQPGGSLRLSCVASGRLSSSNSMA |
| | | | WYRQVPGKRRELVAGITGGGETNYADFVGGRFTISR |
| | | | DNAKNGLYLQLNGLKPEDTAAYYCNFWPPLINYWG |
| | | | QGTQVTVSSAAAYPYDVPDYGSHHHHHH |
| B1 | His6-tag | 21 | DVQLVESGGGLVQPGGSLRLSCVASGRLSSSNSMA |
| | | | WYRQVPGKRRELVAGITGGGETNYADFVGGRFTISR |
| | | | DNAKNGLYLQLNGLKPEDTAAYYCNFWPPLINYWG |
| | | | QGTQVTVSSHHHHHH |
| B1 | untagged | 4 | DVQLVESGGGLVQPGGSLRLSCVASGRLSSSNSMA |
| | | | WYRQVPGKRRELVAGITGGGETNYADFVGGRFTISR |
| | | | DNAKNGLYLQLNGLKPEDTAAYYCNFWPPLINYWG |
| | | | QGTQVTVSS |
| B2 | HA-His6-tag | 18 | QVQLQESGGGLVQAGGSLRLSCAVSGSISSANSMG |
| | | | WYRQAPGKQRDVVAGLTTGGRSHYADSVKGRFTIS |
| | | | RDNAKNTVYLQMNSLKAEDTAVYYCNLWPPVQGYW |
| | | | GQGTQVTVSSAAAYPYDVPDYGSHHHHHH |
| B2 | His6-tag | 22 | DVQLVESGGGLVQAGGSLRLSCAVSGSISSANSMG |
| | | | WYRQAPGKQRDVVAGLTTGGRSHYADSVKGRFTIS |
| | | | RDNAKNTVYLQMNSLKAEDTAVYYCNLWPPVQGYW |
| | | | GQGTQVTVSSHHHHHH |
| B3 | HA-His6-tag | 19 | QVQLQESGGGLVQAGGSLRLSCAVSGRLFSTNAMG |
| | | | WYRQAPGKQRELVAGITGGDRSNYADSVKGRFTIS |
| | | | RDNGKNTLYLQMNSLKPEDTAVYYCNFYPPIVGDYW |
| | | | GQGTQVTVSSAAAYPYDVPDYGSHHHHHH |
| B3 | His6-tag | 23 | DVQLVESGGGLVQAGGSLRLSCAVSGRLFSTNAMG |
| | | | WYRQAPGKQRELVAGITGGDRSNYADSVKGRFTIS |
| | | | RDNGKNTLYLQMNSLKPEDTAVYYCNFYPPIVGDYW |
| | | | GQGTQVTVSSHHHHHH |
| B4 | HA-His6-tag | 20 | QVQLQESGGGLVQVGGSLRLSCVASGFTFSSYYMS |
| | | | WVRQAPGKGLEWVASIYADGDMTYYADSVRGRFTI |
| | | | SRDNAKNTLYLQMNSLKSEDTAVYYCAKDPLPPYHV |
| | | | NQGTQVTVSSAAAYPYDVPDYGSHHHHHH |
| B4 | His6-tag | 24 | DVQLVESGGGLVQVGGSLRLSCVASGFTFSSYYMS |
| | | | WVRQAPGKGLEWVASIYADGDMTYYADSVRGRFTI |
| | | | SRDNAKNTLYLQMNSLKSEDTAVYYCAKDPLPPYHV |
| | | | NQGTQVTVSSHHHHHH |

### Example 2: Knock in Mice

Human FAP knockin (KI) mice were generated by the introduction, via homologous recombination, of a human FAP cDNA (derived from NM_004460.5; SEQ ID NO: 25, coding for SEQ ID NO: 26) after the start codon in exon 1 of the murine FAP gene on chromosome 2 (Gene ID: 14089, NCBI accession number NM_007986.3) in C57BL/6 mice. This disrupts the functional expression of normal murine FAP gene, and instead drives the expression of human FAP under the murine FAP promotor.

In the KI mice, the region from ATG start codon in exon 1 to part of intron 2 of murine FAP is replaced with a “human FAP CDS-polyA” cassette.

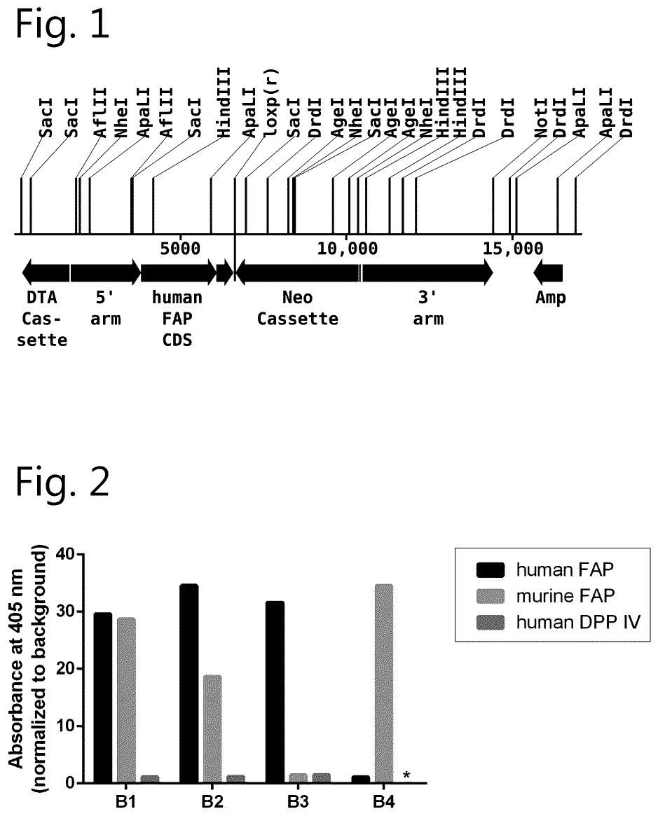

The different elements of the targeting vector are schematically depicted in FIG. 1. The “human FAP CDS-polyA” cassette stretches from “5′arm” to “3′arm”. The 17086 bp sequence of the targeting vector is SEQ ID NO: 27.

The “human FAP CDS-polyA” cassette contains the following relevant elements:
1. The first homology arm (“5′arm” in FIG. 1), encompassing murine FAP 5′ untranslated region and part of exon 1 (until the start codon), is from nucleotides 1704 to 3821. The homology arm is generated by PCR using BAC clone RP23-161B24 or RP24-308110 from a C57BL/6 BAC library as template.
2. The murine FAP exon 1 starts at nucleotide 3652
3. The murine FAP start codon is from nucleotide 3822 to 3824
4. The human FAP coding sequence (derived from NCBI reference sequence NM_004460.5; SEQ ID NO: 25), denoted “human FAP CDS” in FIG. 1, is from nucleotides 3822 to 6104
5. The rabbit beta-globin polyadenylation sequence (“rBGpA” in FIG. 1) is from nucleotides 6105 to 6626
6. The Neomycin-resistance gene (“Neo Cassette” in FIG. 1), used for positive selection of ES cells, is between the rBGpA signal sequence and the second homology arm and is flanked by two LoxP sites (“loxP(r)” in FIG. 1, nucleotides 6640-6673 and 10406-10439)
7. The second homology arm (“3′arm” in FIG. 1), encompassing part of murine FAP intron 2, is from nucleotides 10506 to 14426. The homology arm is generated by PCR using BAC clone RP23-161B24 or RP24-308110 from a C57BL/6 BAC library as template.

The targeting vector additionally contains:
1. The diphtheria toxin A gene (“DTA Cassette” in FIG. 1), used for negative selection of ES cells not undergoing homologous recombination, located upstream of the first homology arm
2. An ampicillin-resistance gene (“Amp” in FIG. 1), used for positive selection of the targeting vector in E. coli

The targeting vector is validated by RFLP analysis and sequencing. The targeting vector is linearized with a NotI DNA restriction enzymes and electroporated in C57BL/6N embryonic stem (ES) cells. Individual clones were selected after positive selection on Neomycin. The genotype of selected ES clones is validated by karyotype analysis, long-range genomic PCR and southern blot analysis.

Selected ES cell clones were micro-injected into C57BL/6 albino blastocysts, which were then re-implanted into CD-1 pseudo-pregnant females. Male F0 founder mice were identified by their coat color and genomic PCR. F0 founder mice were mated with C57BL/6 females. Upon germline transmission, the neomycin cassette self-deletes from the genome. F1 offspring was genotyped by genomic PCR analysis. F1 heterozygous KI mice were generated from 3 different ES clones and were cross-bred. Heterozygous KI mice bred in the expected mendelian genetic ratios of offspring, indicating the sufficiency and lack of toxicity of the human FAP knock in construct. Both heterozygous and homozygous genotypes were viable for at least 1 year without any macroscopic abnormalities.

From published preclinical data, it is indicated that murine FAP is present in several organs and tissues such as blood, lymph nodes, bone, uterus, pancreas, skin and muscle (Puré et al 2018, Oncogene August; 37(32):4343-4357; Keane et al 2014, FEBS Open Bio 4, 43-54). Several reports relate at least part of this elevated uptake to an active shedding of murine FAP (but not human FAP) into the blood stream (Keane et al 2014, FEBS Open Bio 4, 43-54). The results described in Example 5 (Example 5a) using the human FAP knock in mice reveal that this elevated uptake is absent when human/murine cross-reactive FAP-targeting VHH B1 is intravenously administered, indicating the human FAP is not shed to that extend. This means that the human FAP knock in mice described herein accurately represent the human situation.

Additional results described in Example 5 (Example 5b) indicate that human/murine FAP-cross-reactive VHH B1 specifically binds murine FAP expressing fibroblasts in healing wounds in wild type C57BL/6 mice and human FAP expressing fibroblasts in homozygous human FAP knock in mice, while murine FAP-targeting VHH B4 was only able to target murine FAP expressing fibroblasts in healing wounds in wild type mice but not in homozygous human FAP knock in mice. This observation serves as a true validation of the human FAP knock in animal model used in Example 5.

### Example 3: Synthesis of Labelled Compound

His₆-tagged VHHs are radiolabeled with Technetium-99m (^{99m}Tc) for diagnostic purposes, because it emits detectable gamma rays with a photon energy of 140 keV. In short, VHHs are labeled with [^{99m}Tc(H₂O)₃(CO)₃] at their His₆-tag, as described previously in Xavier et al. Methods Mol Biol. 2012; 911:485-90. [⁹⁹mTc(H₂O)₃(CO)₃] was added to 1 mg/ml VHH solution and incubated for 90 min at 37-50° C. After labeling, the ^{99m}Tc-VHH solution was purified on a disposable size-exclusion column pre-equilibrated with PBS to remove unbound [^{99m}Tc(H₂O)₃(CO)₃] and passed through a 0.22 μm filter prior to further use. Quality control (QC) was performed by instant thin layer chromatography.

In the examples below, FAP-targeting His₆-tagged VHHs B1, B2, B3 and B4 (SEQ ID NO: 21-24) have been radiolabeled with the diagnostic radioisotope ^{99m}Tc, and subsequently characterized in vitro and in vivo for diagnostic applications.

VHHs are radiolabeled with ¹³¹I for theranostic purposes, which means that the same radiolabeled VHH can be used for both diagnostic and therapeutic purposes. This can be achieved by applying radioisotopes that emit different types of radiation, that can be used for diagnostic (for example gamma radiation) and therapeutic (for example beta-radiation) purposes. One such a radioisotope is Iodine-131 (¹³¹I), which emits both gamma-rays of about 364 keV and beta-minus particles with a maximum energy of 606 keV. In short, [¹³¹I]SGMIB was synthesized and purified following a procedure adapted from D'Huyvetter M et al. Clin Cancer Res. 2017 Nov. 1; 23(21):6616-6628. Sodium [¹³¹I] iodide was reacted with its trimethylstannyl precursor in acetonitrile for 20 min at RT, after which bisBoc-[¹³¹I]SGMIB was deprotected by the addition of trifluoroacetic acid and subsequently purified using reversed-phase HPLC. Purified [¹³¹I]SGMIB was incubated with 150 μg VHH in 0.1 M borate buffer pH 8.5 for 20 min at RT, conjugating it to lysine side-chain amine reactive groups via nucleophilic substitution, after which [¹³¹I]SGMIB-VHH was purified using a disposable size exclusion column pre-equilibrated with PBS to remove unreacted [¹³¹I]SGMIB, and passed through a 0.22 μm filter prior to further use. Quality control (QC) was performed by instant thin layer chromatography.

In the examples described below, following FAP-targeting VHHs have been radiolabeled with the theranostic radioisotope ¹³¹I, and subsequently characterized in vitro and in vivo for theranostic applications: His₆-tagged VHHs B1, B2, B3 and B4 (SEQ ID NO: 21-24) and untagged VHH B1 (SEQ ID NO: 4) VHHs are also radiolabeled with ¹¹¹In for diagnostic purposes. Indium-111 emits gamma-rays of about 172 and 246 keV. In short, the bifunctional chelator p-SCN-Bn-DOTA was conjugated to lysine side-chain amine reactive groups of the VHH in a 0.05 M sodium carbonate buffer (pH 8.5). After size exclusion purification, resulting VHH-DOTA was reconstituted in 0.1 M ammonium acetate buffer pH 7.0. The necessary amount of ¹¹¹In was added to a test vial containing metal-free 0.1 M ammonium acetate buffer pH 5.0. Then, 25-100 μg of VHH-DOTA was added and incubated for 30 min at 55° C. ¹¹¹1n-DOTA-VHH was purified via size exclusion purification, and passed through a 0.22 μm filter prior to further use. Quality control (QC) was performed by instant thin layer chromatography. In the examples below, FAP-targeting untagged VHH B1 (SEQ ID NO: 4) has been radiolabeled with diagnostic radioisotope ¹¹¹In, and subsequently characterized in vitro and in vivo for diagnostic applications.

VHHs are also radiolabeled with ¹⁷⁷Lu for theranostic purposes (D'Huyvetter et al. (2012) Contrast Media Mol Imaging 7(2):254-264). Lutetium-177 emits both gamma-rays of about 113 and 210 keV and beta-minus particles with a maximum energy of 497 keV. In short, the bifunctional chelator p-SCN-Bn-CHX-A″-DTPA was conjugated to lysine side-chain amine reactive groups of the VHH in a 0.05 M sodium carbonate buffer (pH 8.5). After size exclusion purification, resulting VHH-DTPA was reconstituted in 0.1 M ammonium acetate buffer pH 7.0. The necessary amount of ¹⁷⁷Lu was added to a test vial containing metal-free 0.1 M ammonium acetate buffer pH 5.0. Then, 25-100 μg of VHH-DTPA was added and incubated for 30 min at 55° C. ¹⁷⁷Lu-DTPA-VHH was purified via size exclusion purification, and passed through a 0.22 μm filter prior to further use. Quality control (QC) was performed by instant thin layer chromatography.

In the examples below, FAP-targeting untagged VHH B1 (SEQ ID NO: 4) was radiolabeled with theranostic radioisotope ¹⁷⁷Lu, and subsequently characterized in vitro for theranostic applications.

Finally, VHHs are also radiolabeled with ²²⁵Ac for therapeutic purposes (Pruszynski et al. (2018) Mol Pharm 15(4):1457-1466). Actinium-225 emits alpha particles of 5.8 MeV. In short, the bifunctional chelator p-SCN-Bn-DOTA was conjugated to lysine side-chain amine reactive groups of the VHH in a 0.05 M sodium carbonate buffer (pH 8.5). After size exclusion purification, resulting VHH-DOTA was reconstituted in 0.1 M ammonium acetate buffer pH 7.0. The desired activity of ²²⁵Ac was added to a test vial containing 0.8 M ammonium acetate (pH 5.0) followed by the incubation with VHH-DOTA (25-100 μg) for 90 min at 55° C. The mixture was cooled to RT and quenched with 50 mM DTPA (in 0.8 M ammonium acetate) and Chelex 100 in order to complex any free ²²⁵Ac. ²²⁵Ac-DOTA-VHH was purified via size exclusion purification, and passed through a 0.22 μm filter prior to further use. Quality control (QC) was performed by instant thin layer chromatography.

In the examples below, FAP-targeting untagged VHH B1 (SEQ ID NO: 4) was radiolabeled with the therapeutic radioisotope ²²⁵Ac, and subsequently characterized in vitro for therapeutic applications.

### Example 4: Binding Activities of the VHH or of the Labelled Compound Comprising the VHH

### Example 4a: Recombinant Protein Production

For VHH binding activity testing, human and murine FAP recombinant proteins were produced in HEK293-E cells transiently transfected with endotoxin-free plasmid DNA according to standard protocols such as those in Baldi, et al. (2005) Biotechnol Prog. 21(1):148-53. The extracellular domain of human FAP (amino acid L26 to D760, Uniprot entry Q12884, NCBI reference sequence NP_004451.2) and the extracellular domain of murine FAP (amino acid L26 to D761, Uniprot entry P97321, NCBI reference sequence NP_032012.1) were produced with a N-terminal His₆-tag (SEQ ID NO: 28-29, respectively), using the human Cystatin-S signal peptide (SEQ ID NO: 31) for secretion in the medium. Upon secretion, the signal peptide is proteolytically removed from the recombinant FAP protein. Six days post transfection, conditioned medium containing the recombinant protein was harvested by centrifugation. FAP recombinant protein was purified from the harvested medium via IMAC by batch incubation with Ni Sepharose Excel affinity media (Sigma-Aldrich). After washing with 25 mM Tris, 500 mM NaCl, pH 8.2 and 25 mM Tris, 500 mM NaCl, 20 mM imidazole, pH 8.2, the protein was eluted with 25 mM Tris, 500 mM NaCl, 500 mM imidazole, pH 8.2. Subsequent SEC was performed on a Superdex 200 16/600 column (GE Healthcare), equilibrated in PBS.

The recombinant FAP proteins formed a homodimer in solution, as this is a prerequisite for its enzymatic activity (Aertgeerts K et al. (2005) J Biol Chem 280(20):19441-4), as measured in Example 4d.

### Example 4b: ELISA: Specific Binding: Human/Murine FAP, DPP IV

This part of the example, describes the specific binding of VHHs B1, B2, B3 and B4 to the extracellular domain of human and/or murine FAP (NCBI reference sequence NP_004451.2 and NP_032012.1, resp.) and absence of binding to the extracellular domain of human DPP IV (NCBI reference sequence NP_001926.2), as tested in ELISA. While DPPIV and FAP both belong to the family of dipeptidyl peptidases, DPP IV is the closest homologue of FAP, sharing about 50% homology in its amino acid sequence (Juillerat-Jeanneret L et al. (2017). Expert Opin Ther Targets 21(10):977-991).

The day before measurement, 0.1 μg recombinant protein at a concentration of 1 μg/mL in 100 mM NaHCO₃, pH 8.2 was coated in a 96-well ELISA plate (Nunc MaxiSorp). Per VHH clone also a blank coated well was foreseen (only buffer). The wells were overcoated with protein-free T20 (PBS) blocking buffer (Pierce). After washing with PBS, pH7.4, 0.05% Tween, VHH-containing bacterial freeze-thaw extract was added to every well. Binding of HA-His₆-tagged VHHs was detected by using mouse anti-HA.11 epitope tag (clone 16B12, Biolegend) as the primary Ab and goat anti-mouse IgG (whole molecule) alkaline phosphatase conjugate (Sigma-Aldrich) as the secondary Ab, with thorough washing with PBS, pH 7.4, 0.05% Tween in between. Signals were developed using phosphatase substrate (Sigma-Aldrich) in AP blot buffer (100 mM NaCl, 50 mM MgCl₂, 100 mM Tris, pH 9.5). The absorbance was determined at 405 nm using an absorbance microplate reader (Molecular Devices). Per clone the ratio was determined between the absorbance in the antigen-coated well versus the well without antigen.

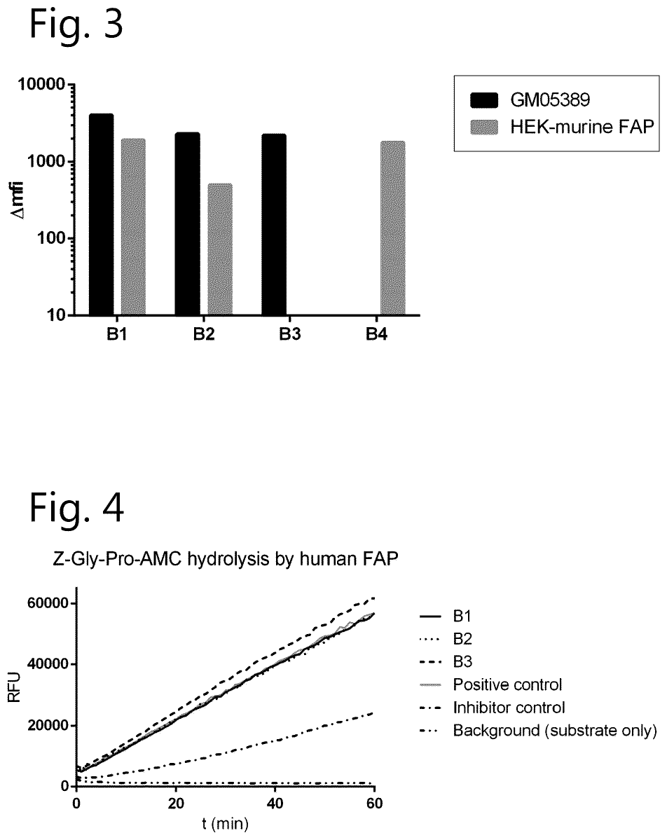

As show in FIG. 2, VHHs B1, B2 and B3 (SEQ ID NO: 17-19) showed signal-to-background ratios greater than 25 for wells coated with human FAP recombinant protein (SEQ ID NO: 28), confirming specific binding to human FAP. VHHs B1 and B2 also showed to be able to specifically bind murine FAP recombinant protein (SEQ ID NO: 29), while this was not the case for VHH B3. In its turn, VHH B4 (SEQ ID NO: 20) showed a signal-to-background ratio greater than 30 for wells coated with murine FAP recombinant protein, confirming specific binding to murine FAP, while it was not binding human FAP. These experiments demonstrate that VHHs B1 and B2 are murine/human FAP-crossreactive, VHH B3 is human FAP-specific, and VHH B4 is murine FAP-specific.

All human FAP-binding VHHs showed absence of binding to human DPPIV (Sino Biological), with signal-to-background ratios around 1.

### Example 4c: Flow Cytometry: GM05389 and HEK-Murine FAP

This part of the example describes the ability of the VHHs to target the naturally expressed receptor on the human FAP-expressing fibroblast cell line GM05389 (obtained from the NIGMS Human Genetic Cell Repository at the Coriell Institute for Medical Research) and the murine FAP transfected cell line HEK293 (obtained from Jonathan D. Cheng, Fox Chase Cancer Center, Philadelphia, PA; described in Cheng, J. D., et al. (2002) Cancer Research 62(16): 4767-4772).

The cell binding was tested by flow cytometry experiments. Per test condition 1×10⁵ to 2×10⁵ cells, washed in PBS, 0.5% BSA, were pelleted in the well of a 96-well U-bottom plate. The cell pellets were resuspended and incubated with the VHH-containing bacterial freeze-thaw extracts (diluted in PBS, 0.5% BSA). Binding of HA-His₆-tagged VHHs was detected by using mouse anti-HA.11 epitope tag (clone 16B12, Biolegend) as the primary detection antibody and PE-conjugated rat anti-mouse IgG1 (clone A85-1, BD Pharmingen) as the secondary detection antibody, with washing with PBS, 0.5% BSA in between, and finally resuspended in PBS, 0.5% BSA.

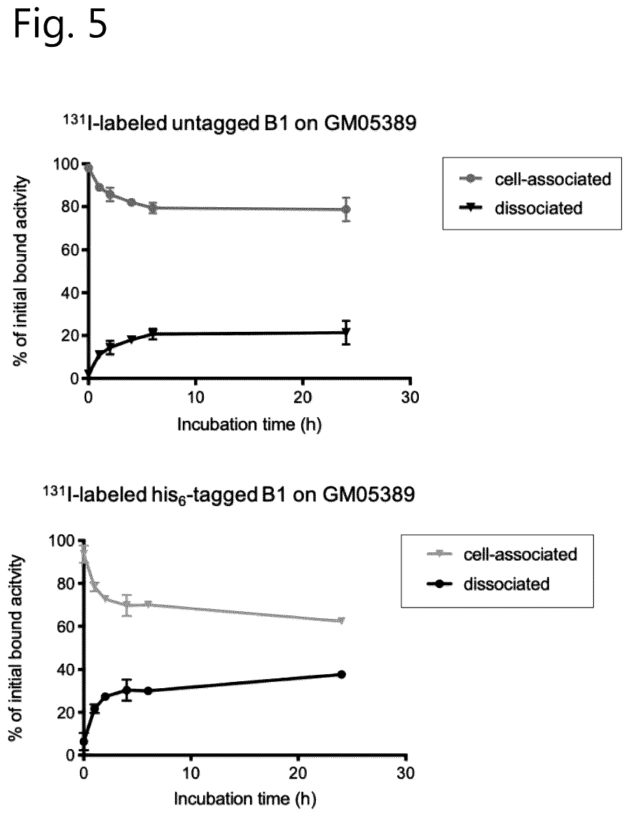

Flow cytometry was performed on a FACS Canto II (BD Biosciences). Data were analyzed using FlowJo software. Based on the forward scatter-side scatter plot a single cell gate was drawn. The median fluorescence intensity was determined on a histogram for the phycoerythrin signal of the single cells. The difference in median fluorescence intensity (A mfi) was calculated relative to a test condition without incubation of VHHs (cells+primary Ab+secondary Ab), and are shown in FIG. 3.

In accordance with the ELISA results in Example 4b using recombinant proteins, VHHs B1, B2 and B3 (SEQ ID NO: 17-19) were able to bind the human FAP-expressing cell line GM05389, while VHHs B1, B2 and B4 (SEQ ID NO: 20) were able to bind the murine FAP transfected cell line HEK293.

### Example 4d: Enzymatic Activity

This part of the example describes the non-inhibitory effect on human FAP dipeptidyl peptidase enzymatic activity upon VHH binding.

The human FAP enzymatic activity was measured using the fluorogenic substrate benzyloxycarbonyl-Gly-Pro-7-amido-4-methylcoumarin (Z-Gly-Pro-AMC; Bachem).

Human FAP recombinant protein (SEQ ID NO: 28) was diluted to 200 ng/ml in assay buffer (50 mM Tris-HCl, 1 M NaCl, 0.1% BSA, pH 7.5) in a black 96-well flat bottom plate, in absence or presence of 1 μM HA-His₆-tagged VHH. As an inhibitor control, 1 μM Talabostat mesylate (ApexBio) was added instead of VHH. After 1 h incubation to reach binding equilibrium, Z-Gly-Pro-AMC substrate was added at a final concentration of 50 μM. Enzymatic conversion of the substrate into Z-Gly-Pro and 7-amino-4-methylcoumarin (AMC) was followed using a fluorescence microplate reader (BioTek) with excitation at 380 nm and emission detection at 460 nm. Fluorescence was measured every minute during 1 h. The slope of the curves depicted in FIG. 4 corresponds to the rate of enzymatic activity.

Human FAP binding VHHs B1, B2 and B3 (SEQ ID NO: 17-19) showed no effect on the human FAP dipeptidyl peptidase enzymatic activity, converting Z-Gly-Pro-AMC into Z-Gly-Pro and AMC. The rate of enzymatic activity in presence of VHH corresponded to the condition without VHH (positive control), while co-incubation with Talabostat mesylate (inhibitor control) showed a reduced substrate hydrolysis rate resulting in lower fluorescence levels.

### Example 4e: Antigen Binding Kinetics

This part of the example describes the determination of antigen binding kinetics.

To this extent, human and murine FAP recombinant proteins (SEQ ID NO: 28-29) were biotinylated in PBS buffer using a 5× molar excess of sulfo-NHS-SS-biotin (Thermo Fisher Scientific). Excess of biotin was eliminated using a Zebaspin desalting column (Thermo Fisher Scientific) equilibrated in PBS.

The kinetic parameters of antigen binding by purified VHHs were determined via bio-layer interferometry with an Octet RED96 instrument (ForteBio). The assay was performed at 25° C. with shaking at 1000 rpm. Streptavidin coated SA sensors were equilibrated in assay buffer (HBS supplemented with 0.5% BSA, 0.1% Tween), before loading for 5 min with the biotinylated FAP recombinant protein at a concentration of 5 μg/ml. His₆-tagged VHHs were analyzed in a threefold serial dilution from 30 nM to 0.4 nM in assay buffer (except for VHH B3 binding murine FAP recombinant protein, for which a fivefold serial dilution from 1000 nM to 1.6 nM was employed). First the baseline was measured for 180 s in assay buffer, followed by 5 subsequent association phases of 180 s with the VHH preparations in increasing order of concentration, with 75 s intermediate dissociation phases in assay buffer in between, and a final dissociation phase in assay buffer of 30 min. In every assay a reference sensor loaded with FAP recombinant protein was included that was incubated in assay buffer during the association phases.

Binding curves were exported and analyzed using the BIAevaluation analysis software (GE Healthcare). First the y-axes of the different curves were aligned to the median of the last 5 s of the baseline measurement. Subsequently, the sensorgram of the reference sensor was subtracted from all other sensorgrams. The resulting binding curve was fitted using a general fit with the ‘kinetic titration with drift’ binding model (1:1 (antigen:analyte) model; Karlsson, R. et al (2006), Analyzing a kinetic titration series using affinity biosensors. Analytical Biochemistry 349: 136-147), with Rmax fitted global, RI local and drift set to 0.

Table 7 gives an overview of the kinetic parameters of antigen binding for His₆-tagged VHHs B1, B2, B3 and B4 (SEQ ID NO: 21-24), as determined by bio-layer interferometry with biotinylated human or murine FAP recombinant protein that was loaded on the sensor.

VHHs B1, B2 and B3 showed at least subnanomolar affinities for binding human FAP with equilibrium dissociation constants (K_{D}) that were smaller than 1 nM. In particular, best binding characteristics were observed for VHH B1 with K_{D} value <50 μM, and dissociation reaction rate constant (k_{d})<10⁻⁵ s⁻¹. VHHs B1 and B4 were able to bind murine FAP with K_{D} values <1 nM. VHH B2 had a moderate affinity (K_{D}) of 24 nM for murine FAP.

TABLE 7
Kinetic binding parameters kₐ, k_{d} and K_{D}
of purified, His₆-tagged VHHs B1, B2, B3 and B4.

| | kₐ (M⁻¹ s⁻¹) | k_{d} (s⁻¹) | K_{D} (M) |
|---|---|---|---|
| Human FAP | | | |
| B1 | 7.0E+05 | 2.7E−05 | 37.8E−12 |
| B2 | 4.2E+05 | 3.1E−05 | 74.4E−12 |
| B3 | 3.4E+05 | 5.4E−05 | 160.0E−12 |
| B4 | Not determined | | |
| Murine FAP | | | |
| B1 | 1.9E+06 | 1.4E−03 | 742.0E−12 |
| B2 | 3.4E+05 | 8.2E−03 | 24.3E−9 |
| B3 | Not binding | | |
| B4 | 1.1E+06 | 5.6E−04 | 512.0E−12 |

### Example 4f. In Vitro and In Vivo Binding Activity of Radiolabelled VHHs to FAP

Next, we describe the in vitro and in vivo human and murine FAP binding potential of different VHHs after radiolabelling with the diagnostic radioisotope ^{99m}Tc. EC50 values of the different ^{99m}Tc-labeled VHHs were determined on human FAP-expressing HEK-293 and GM05389 cells as well as on murine FAP-expressing HEK-293 cells (GM05389 was obtained from the NIGMS Human Genetic Cell Repository at the Coriell Institute for Medical Research; and the FAP transfected HEK293 cell lines were obtained from Jonathan D. Cheng, Fox Chase Cancer Center, Philadelphia, PA; described in Cheng, J. D., et al. (2002) Cancer Research 62(16): 4767-4772). His₆-tagged human/murine FAP-targeting VHHs were radiolabelled with the diagnostic radioisotope ^{99m}Tc, according to the radiochemical procedure described in Example 3.

Next, the different cell lines were incubated with serial dilutions with concentrations ranging from 0 to 300 nM of the different ^{99m}Tc-labelled His₆-tagged VHHs. A 100-fold excess of the corresponding unlabeled VHH was added in parallel to saturate the human or murine FAP proteins on the cancer cell surface, to assess non-specific binding. The resulting EC50 values for each of the VHHs are depicted in Table 8. From these data it is indicated that both VHHs B1 and B2 maintain their ability to bind both human and murine FAP cell surface proteins after radiolabeling with ^{99m}Tc. Non targeting control VHH R3B23 (SEQ ID NO: 32; Lemaire et al. (2014) Leukemia 28(2):444-447) did not reveal any relevant binding towards any of the three cell lines evaluated. We confirmed that ^{99m}Tc-labeled VHH B4 only binds murine FAP cell surface protein, while VHH B3 only binds human FAP cell surface molecules in vitro.

TABLE 8
The resulting EC50 of VHHs on three different cell
lines: human FAP-expressing HEK-293 and GM05389
cells as well as on murine FAP-expressing HEK-293
cells. Data are expressed as mean ± SD (n = 3).

| | EC50 (mean ± SD, nM) | | |
|---|---|---|---|
| | GM05389 | HEK-293 | HEK-293 |
| VHHs | human FAP | human FAP | murine FAP |
| B1 | 0.5 ± 0.1 | 0.6 ± 0.3 | 5.8 ± 0.4 |
| B2 | 3.2 ± 0.5 | 13.0 ± 3.5 | 139.1 ± 30.0 |
| B3 | 2.7 ± 0.2 | 2.4 ± 0.4 | Not binding |
| B4 | Not binding | Not binding | 7.6 ± 0.5 |
| R3B23 | Not binding | Not binding | Not binding |

The different ^{99m}Tc-labeled human-only, murine-only or human/murine cross-reactive FAP-targeting VHHs were evaluated in vivo in normal female C57BL/6 mice via dissection studies. Mice (n=3 per VHH) were intravenously injected with about 1 mCi (±4 μg) ^{99m}Tc-labeled VHHs. Next, blood was taken by hearth puncture after which the mice were euthanized by cervical dislocation at 1 h post injection. The mice were dissected and different organs and tissues were collected. Organs and tissues of interest were weighed and measured for radioactivity using a gamma counter, along with injection standards. Results were expressed as % of injected activity (IA)/g tissue.

From these data it is indicated that VHHs B1, B2 and B4 maintained their ability to bind murine FAP after radiolabeling with ^{99m}Tc, with elevated retention of radioactivity in several organs and tissues such as blood, lymph nodes, bone, uterus, pancreas, skin and muscle that express and/or contain circulating FAP (Keane et al 2014, FEBS Open Bio 4, 43-54). The extent of retention (as a measure for binding murine FAP) is in line with their corresponding binding affinity for murine FAP (B4>B1>B2). Non-targeting control VHH R3B23 did not reveal any relevant targeting of murine FAP. ^{99m}Tc-labeled, His₆-tagged, human FAP-targeting VHH B3 revealed a biodistribution in mice that is typical for a non-targeting radiolabeled His₆-tagged VHH (Table 9). Low radioactive signal was measured in all organs and tissues except for kidneys.

TABLE 9
Uptake in different organs and tissues obtained from dissections 1 h after injection
of ^{99m}Tc-labeled His₆-tagged VHHs B2, B1, B3, B4 and non-targeting control R3B23 in
normal female C57BL/6 mice. Data expressed as % IA/g and presented as mean ± SD (n = 3).

| | ^{99m}Tc-B2 | | ^{99m}Tc-B1 | | ^{99m}Tc-B3 | | ^{99m}Tc-B4 | | ^{99m}Tc-R3B23 | |
|---|---|---|---|---|---|---|---|---|---|---|
| Organ/tissue | Mean | SD | Mean | SD | Mean | SD | Mean | SD | Mean | SD |
| Blood | 0.97 | 0.09 | 2.52 | 0.03 | 0.59 | 0.11 | 2.74 | 0.18 | 0.89 | 0.30 |
| Lymph nodes | 1.27 | 0.19 | 4.54 | 0.23 | 0.56 | 0.09 | 3.30 | 0.35 | 0.60 | 0.27 |
| Heart | 0.38 | 0.04 | 0.78 | 0.01 | 0.29 | 0.08 | 0.91 | 0.07 | 0.36 | 0.09 |
| Lung | 0.84 | 0.07 | 1.35 | 0.26 | 0.73 | 0.12 | 1.30 | 0.22 | 0.80 | 0.13 |
| Liver | 1.18 | 0.14 | 1.59 | 0.14 | 0.77 | 0.04 | 0.90 | 0.08 | 0.54 | 0.06 |
| Pancreas | 0.39 | 0.04 | 1.27 | 0.13 | 0.21 | 0.04 | 1.49 | 0.13 | 0.23 | 0.01 |
| Spleen | 0.50 | 0.04 | 0.82 | 0.15 | 0.30 | 0.03 | 0.67 | 0.05 | 0.29 | 0.01 |
| Adrenals | 1.05 | 0.23 | 2.75 | 0.40 | 0.55 | 0.12 | 2.13 | 0.29 | 0.51 | 0.43 |
| Kidney | 242.15 | 15.68 | 244.68 | 29.15 | 408.44 | 95.18 | 50.83 | 5.97 | 334.43 | 114.92 |
| Stomach | 1.50 | 0.06 | 1.62 | 0.17 | 0.88 | 0.08 | 1.05 | 0.19 | 1.09 | 0.16 |
| Small intestine | 0.55 | 0.06 | 1.67 | 0.07 | 0.31 | 0.05 | 0.89 | 0.18 | 0.35 | 0.08 |
| Large intestine | 0.53 | 0.05 | 1.01 | 0.09 | 0.49 | 0.12 | 0.89 | 0.05 | 0.43 | 0.08 |
| Uterus | 1.51 | 0.31 | 2.69 | 0.72 | 0.72 | 0.23 | 2.86 | 0.55 | 0.62 | 0.05 |
| Skin | 0.66 | 0.13 | 1.27 | 0.03 | 0.58 | 0.07 | 1.89 | 0.20 | 0.46 | 0.16 |
| Muscle | 0.46 | 0.21 | 0.94 | 0.16 | 0.22 | 0.12 | 1.38 | 0.17 | 0.26 | 0.12 |
| Bone | 1.26 | 0.13 | 3.41 | 0.45 | 0.22 | 0.04 | 3.81 | 0.87 | 0.45 | 0.39 |
| Brain | 0.05 | 0.01 | 0.08 | 0.02 | 0.03 | 0.02 | 0.09 | 0.02 | 0.02 | 0.01 |

To assess whether the elevated uptake observed in several organs and tissues was due to specific targeting, ^{99m}Tc-labeled VHH B1 was administered alone and in combination with a 100 fold molar excess of unlabeled VHH B1 to healthy female C57BL/6 mice, after which its biodistribution was assessed via dissection studies. Mice (n=3 per condition) were intravenously injected with about 1 mCi (±4 μg) ^{99m}Tc-labeled VHHs. Unlabelled VHH, or an equal amount of vehicle solution, was administered 30 minutes prior to tracer administration. Next, blood was taken by heart puncture after which the mice were euthanized by cervical dislocation at 1 h post injection. The mice were dissected, isolated organs and tissues of interest were weighed and measured for radioactivity using a gamma counter, along with injection standards. Results were expressed as % of injected activity (IA)/g tissue. Results show that elevated uptake in blood, lymph nodes, bone, uterus, pancreas, skin and muscle is completely reduced by co-administration with unlabeled VHH B1 (Table 10), indicating that the elevated uptake is due to the specific targeting capacity of VHH B1, rather than non-specific retention.

TABLE 10
Uptake in different organs and tissues obtained from dissections
1 h after injection of ^{99m}Tc-labeled His-tagged VHH B1
alone and together with a 100 fold molar excess of unlabeled
VHH in normal female C57BL/6 mice. Data expressed as %
IA/g and presented as mean ± SD (n = 3).

| | |
|---|---|
| | ^{99m}Tc-B1 + 100 fold |
| ^{99m}Tc-B1 | excess unlabeled B1 |

| Organ/tissue | Mean | SD | Mean | SD |
|---|---|---|---|---|
| Blood | 1.90 | 0.27 | 0.78 | 0.10 |
| Lymph nodes | 3.19 | 0.28 | 0.64 | 0.23 |
| Heart | 0.75 | 0.05 | 0.37 | 0.15 |
| Lung | 1.01 | 0.13 | 0.72 | 0.09 |
| Galbladder | 3.01 | 0.52 | 1.32 | 0.52 |
| Liver | 1.05 | 0.20 | 0.81 | 0.01 |
| Pancreas | 1.39 | 0.13 | 0.24 | 0.01 |
| Spleen | 0.59 | 0.09 | 0.35 | 0.03 |
| Adrenals | 2.39 | 0.42 | 0.75 | 0.02 |
| Kidney | 198.25 | 31.22 | 233.90 | 14.29 |
| Stomach | 1.19 | 0.11 | 0.87 | 0.03 |
| Small intestine | 0.92 | 0.40 | 0.61 | 0.09 |
| Large intestine | 0.94 | 0.11 | 0.45 | 0.07 |
| Uterus | 2.83 | 0.45 | 0.93 | 0.08 |
| Skin | 1.92 | 0.26 | 0.75 | 0.06 |
| Muscle | 1.15 | 0.12 | 0.29 | 0.02 |
| Bone | 2.54 | 0.23 | 0.35 | 0.06 |
| Brain | 0.07 | 0.00 | 0.03 | 0.00 |

### Example 5

In this example we describe the potential of human-only or human/murine cross-reactive FAP-targeting VHHs to measure relevant human or murine FAP-expression after radiolabelling with the diagnostic radioisotope ^{99m}Tc (as described in example 3) in both normal C57BL/6 mice and homozygous human FAP knock in mice, and in mice that undergo wound healing.

### Example 5a

In a first part of this example, the biodistribution of human/murine cross-reactive FAP-targeting VHH B1, murine FAP-targeting VHH B4 and non-targeting control VHH R3B23 was assessed in both normal female C57BL/6 mice as well as in homozygous human FAP knock in mice. All mice were intravenously injected in the tail vein with about 1 mCi (±4 μg) of ^{99m}Tc-labelled VHHs. Next, mice were euthanized by cervical dislocation after 1.5 h, dissected after which different organs and tissues were collected. Organs and tissues of interest were weighed and measured for radioactivity using an automatic gamma counter, along with injection standards. Results were expressed as % of injected activity (IA)/g tissue.

Results indicate elevated uptake in blood, lymph nodes, bone, uterus, pancreas, skin and muscle of normal mice after i.v. administration of ^{99m}Tc-labeled B1 and B4, both targeting murine FAP (Table 11). The elevated uptake is absent in the case of ^{99m}Tc-R3B23. This observation can be explained by the fact that in normal mice, murine FAP is actively shed into the blood stream, which leads to elevated tracer uptake in blood and highly perfused organs and tissues (Keane et al 2014, FEBS Open Bio 4, 43-54). This elevated uptake is absent when human/murine cross-reactive FAP-targeting VHH B1 is i.v. administered to homozygote human FAP knock in mice, indicating that the human FAP is not shed to that extent, which accurately represents the human situation.

TABLE 11
Uptake in different organs and tissues obtained from dissections 1.5 h after injection
of ^{99m}Tc-labeled His₆-tagged VHHs in both wild type C57BL/6 mice (WT) and homozygous human
FAP knock in mice (KI). Data expressed as % IA/g and presented as mean ± SD (n = 3).

| | ^{99m}Tc-B1 | | ^{99m}Tc-B1 | | ^{99m}Tc-B4 | | ^{99m}Tc-B4 | | ^{99m}Tc-R3B23 | | ^{99m}Tc-R3B23 | |
|---|---|---|---|---|---|---|---|---|---|---|---|---|
| | in WT mice | | in KI mice | | in WT mice | | in KI mice | | in WT mice | | in KI mice | |
| Organ | Mean | SD | Mean | SD | Mean | SD | Mean | SD | Mean | SD | Mean | SD |
| Blood | 1.81 | 0.07 | 0.77 | 0.07 | 2.74 | 0.18 | 0.54 | 0.03 | 0.89 | 0.3 | 0.5 | 0.07 |
| Lymph nodes | 3.26 | 1.63 | 0.23 | 0.03 | 3.3 | 0.35 | 0.23 | 0.07 | 0.6 | 0.27 | 0.14 | 0.09 |
| Pancreas | 1.38 | 0.11 | 0.2 | 0.02 | 1.49 | 0.13 | 0.14 | 0.01 | 0.23 | 0.01 | 0.12 | 0.01 |
| Uterus | 2.07 | 1.02 | 0.87 | 0.06 | 2.86 | 0.55 | 0.76 | 0.14 | 0.62 | 0.05 | 0.61 | 0.14 |
| Skin | 1.68 | 0.74 | 0.89 | 0.17 | 189 | 0.2 | 0.63 | 0.12 | 0.46 | 0.16 | 0.64 | 0.21 |
| Muscle | 0.65 | 0.24 | 0.2 | 0.03 | 1.38 | 0.17 | 0.15 | 0.04 | 0.26 | 0.12 | 0.11 | 0.01 |
| Bone | 2.2 | 0.51 | 0.42 | 0.02 | 3.81 | 0.87 | 0.25 | 0.07 | 0.45 | 0.39 | 0.13 | 0.01 |

### Example 5b

Secondly, it is known that fibroblasts are involved in the process of wound healing (Giesel et al 2019 J Nucl Med 60(3):386-392; Dienus K et al. Arch Dermatol Res. 2010 December; 302(10):725-31; Gao Y et al. Fa Yi Xue Za Zhi. 2009 December; 25(6):405-8). We took advantage from this phenomenon to further support the diagnostic potential of ^{99m}Tc-labeled VHHs to visualise FAP-expressing fibroblasts. To this, both wild type C57BL/6 mice and homozygous human FAP knock in mice received a small incision on the lower back, which was sutured immediately. One day later, all mice were intravenously injected in the tail vein with about 1 mCi (±4 μg) of ^{99m}Tc-labelled VHHs. Next, SPECT/CT was performed 1 h after tracer administration using a Vector⁺/CT MILabs system followed by necropsy study after 1.5 h. SPECT/CT imaging was performed using a SPECT collimator and a spiral scan mode of six bed positions (2.5 min per position). For CT, a normal scan mode of only one position was used. The obtained SPECT data are reconstructed with a 0.4 voxel size, 2 subsets and 4 iterations, after which images are fused and corrected for attenuation based on the CT scan. Images are analyzed using a medical image data analysis tool (AMIDE). Uptake values in a selection of organs and tissues were analyzed and expressed as % injected activity per cm³ (% IA/cc).

Next, mice were euthanized by cervical dislocation after 1.5 h, dissected after which different organs and tissues were collected. Organs and tissues of interest were weighed and measured for radioactivity using an automatic gamma counter, along with injection standards. Results were expressed as % of injected activity (IA)/g tissue.

The results described in Tables 7 and 8 below indicate that human/murine FAP-crossreactive VHH B1 specifically binds in healing wounds in vivo murine FAP expressing fibroblasts in wild type C57BL/6 mice and human FAP expressing fibroblasts in homozygous human FAP knock in mice. The uptake value obtained via dissections was significantly higher compared to what was measured for murine FAP-specific VHH B4 (p=0.0044) and non-targeting control VHH R3B23 (p=0.0055) in the healing wound of the homozygous human FAP knock in mice. The uptake of human/murine FAP-crossreactive B in healing wounds in knock-in and wild-type mice respectively, did not significantly differ (p=0.1756), as determined via the unpaired t-test.

TABLE 12
Uptake in different organs and tissues obtained from
SPECT/CT imaging 1 h after injection of ^{99m}Tc-labeled
His₆-tagged VHHs in both wild type C57BL/6 mice (WT)
and homozygote human FAP knock in mice (KI). Data expressed
as % IA/cc and presented as mean ± SD (n = 3).

| | ^{99m}Tc-B1 | | ^{99m}Tc-B1 | | ^{99m}Tc-B4 | | ^{99m}Tc-R3B23 | |
|---|---|---|---|---|---|---|---|---|
| | in WT mice | | in KI mice | | in KI mice | | in KI mice | |
| Organ | Mean | SD | Mean | SD | Mean | SD | Mean | SD |
| Heart | 0.80 | 0.03 | 0.22 | 0.04 | 0.23 | 0.03 | 0.27 | 0.07 |
| Lung | 0.65 | 0.04 | 0.15 | 0.05 | 0.17 | 0.01 | 0.15 | 0.04 |
| Kidney | 78.53 | 2.43 | 101.66 | 8.94 | 22.54 | 2.72 | 84.70 | 29.32 |
| Joint | 1.72 | 0.12 | 0.27 | 0.06 | 0.22 | 0.03 | 0.19 | 0.06 |
| Bone | 0.82 | 0.09 | 0.13 | 0.07 | 0.10 | 0.08 | 0.15 | 0.06 |
| Wound | 1.21 | 0.01 | 0.45 | 0.24 | 0.13 | 0.03 | 0.15 | 0.03 |
| Muscle | 0.50 | 0.02 | 0.10 | 0.01 | 0.10 | 0.06 | 0.15 | 0.06 |
| Liver | 0.76 | 0.04 | 0.33 | 0.05 | 0.26 | 0.01 | 0.28 | 0.02 |

TABLE 13
Uptake in different organs and tissues obtained from dissections 1.5
h after injection of ^{99m}Tc-labeled His₆-tagged VHHs in both wild
type C57BL/6 mice (WT) and homozygote human FAP knock in mice (KI).
Data expressed as % IA/g and presented as mean ± SD (n = 3).

| | ^{99m}Tc-B1 | | ^{99m}Tc-B1 | | ^{99m}Tc-B4 | | ^{99m}Tc-R3B23 | |
|---|---|---|---|---|---|---|---|---|
| | in WT mice | | in KI mice | | in KI mice | | in KI mice | |
| Organ | Mean | SD | Mean | SD | Mean | SD | Mean | SD |
| Blood | 1.81 | 0.07 | 0.77 | 0.07 | 0.54 | 0.03 | 0.50 | 0.07 |
| Lymph nodes | 3.26 | 1.63 | 0.23 | 0.03 | 0.23 | 0.07 | 0.14 | 0.09 |
| Thymus | 0.61 | 0.25 | 0.22 | 0.05 | 0.12 | 0.03 | 0.13 | 0.02 |
| Heart | 0.75 | 0.05 | 0.34 | 0.03 | 0.24 | 0.03 | 0.22 | 0.01 |
| Lung | 1.06 | 0.17 | 0.85 | 0.05 | 0.73 | 0.05 | 0.53 | 0.02 |
| Galbladder | 0.92 | 0.20 | 0.86 | 0.40 | 0.30 | 0.04 | 1.27 | 0.74 |
| Liver | 1.27 | 0.36 | 0.82 | 0.21 | 0.57 | 0.03 | 0.40 | 0.02 |
| Pancreas | 1.38 | 0.11 | 0.20 | 0.02 | 0.14 | 0.01 | 0.12 | 0.01 |
| Spleen | 0.61 | 0.11 | 0.39 | 0.03 | 0.29 | 0.01 | 0.23 | 0.02 |
| Adrenals | 1.91 | 0.39 | 0.48 | 0.09 | 0.39 | 0.13 | 0.28 | 0.00 |
| Kidney | 270.42 | 138.31 | 399.96 | 39.56 | 76.80 | 9.53 | 334.82 | 17.17 |
| Stomach | 0.87 | 0.16 | 0.51 | 0.06 | 0.60 | 0.24 | 0.37 | 0.03 |
| Small intestine | 0.55 | 0.03 | 0.37 | 0.04 | 0.26 | 0.01 | 0.24 | 0.02 |
| Large intestine | 0.82 | 0.07 | 0.43 | 0.04 | 0.30 | 0.02 | 0.34 | 0.02 |
| Ovaries | 1.90 | 0.42 | 0.73 | 0.13 | 0.68 | 0.18 | 0.64 | 0.23 |
| Uterus | 2.07 | 1.02 | 0.87 | 0.06 | 0.76 | 0.14 | 0.61 | 0.14 |
| Skin | 1.68 | 0.74 | 0.89 | 0.17 | 0.63 | 0.12 | 0.64 | 0.21 |
| Muscle | 0.65 | 0.24 | 0.20 | 0.03 | 0.15 | 0.04 | 0.11 | 0.01 |
| Bone | 2.20 | 0.51 | 0.42 | 0.02 | 0.25 | 0.07 | 0.13 | 0.01 |
| Joint | 3.51 | 0.81 | 0.83 | 0.04 | 0.41 | 0.05 | 0.33 | 0.03 |
| Brain | 0.07 | 0.02 | 0.03 | 0.00 | 0.02 | 0.00 | 0.02 | 0.00 |
| Wound | 2.36 | 0.82 | 1.17 | 0.10 | 0.59 | 0.05 | 0.58 | 0.10 |

### Example 6: Diagnostic Use of the Radiolabelled VHHs

In this example we describe the diagnostic potential of human-only or human/murine cross-reactive FAP-targeting VHHs by investigating their ability to visualise relevant human or murine FAP-expression after radiolabelling with the diagnostic radioisotopes ^{99m}Tc and ¹¹¹In (as described in example 3). The diagnostic potential is confirmed in athymic nude mice bearing human MDA-MB-231 tumors with naturally infiltrating cancer-associated fibroblasts expressing murine FAP and in athymic nude mice bearing HEK-293 tumors expressing human FAP receptor.

### Example 6a

In a first part of this example, the diagnostic potential of ^{99m}Tc-labeled human/murine cross-reactive FAP-targeting VHHs B1 and B2, along with murine FAP-specific VHH B4, a non-targeting control VHH R3B23, and human FAP-targeting VHH B3 was evaluated in athymic nude mice bearing MDA-MB-231 tumors with naturally infiltrating cancer-associated fibroblasts expressing murine FAP.

To this, athymic nude mice (n=3 per VHH) were inoculated with 10×10⁶ MDA-MB-231 cells in Matrigel. After validation of tumor growth (mean size of about 200 mm³), mice were intravenously injected in the tail vein with about 1 mCi (±4 μg) of ^{99m}Tc-labelled VHHs. Next, SPECT/CT was performed 1 h after tracer administration using a Vector⁺/CT MILabs system followed by necropsy study after 1.5 h, as described above.

Results indicate that both murine FAP-specific VHH B4 and human/murine FAP-crossreactive VHH B1 specifically accumulated in MDA-MB-231 tumors, while elevated uptake in this tissue was lower for the human/murine FAP-crossreactive VHH B2, and absent for ^{99m}Tc-labeled non-targeting control VHH R3B23 and for the human FAP-specific VHH B3, which is in line with the corresponding affinity of these VHHs for murine FAP. The specific accumulation of both murine FAP-specific VHH B4 and human/murine FAP-crossreactive VHH B1 was significantly higher compared to the additional evaluated VHHs, as indicated by one-way ANOVA-multiple comparisons (p<0,05) (Tables ##9 and ##10). This indicates that the VHHs are capable of visualizing cancer-associated fibroblasts in a normal mouse by means of targeting murine FAP-receptor. This is an important finding, as we know that VHH B1 also binds human FAP with high binding affinity. Therefore it is appropriate to assume that cross-reactive human/murine FAP-targeting VHH B1 will also target human FAP-expressing tumor-associated fibroblasts in a human situation.

TABLE 14
Uptake in different organs and tissues obtained from SPECT/CT imaging 1 h
after injection of ^{99m}Tc-labeled His₆-tagged VHHs in athymic nude mice
bearing MDA-MB-231 tumors. Data expressed as % IA/cc and presented as
mean ± SD (n = 3).

| | ^{99m}Tc-B2 | | ^{99m}Tc-B1 | | ^{99m}Tc-R3B23 | | ^{99m}Tc-B3 | | ^{99m}Tc-B4 | |
|---|---|---|---|---|---|---|---|---|---|---|
| Organ/tissue | Mean | SD | Mean | SD | Mean | SD | Mean | SD | Mean | SD |
| Kidney | 65.02 | 2.22 | 58.25 | 11.32 | 71.82 | 4.49 | 80.24 | 2.86 | 15.15 | 1.65 |
| Heart | 0.37 | 0.07 | 0.65 | 0.09 | 0.27 | 0.09 | 0.14 | 0.02 | 0.92 | 0.14 |
| Lung | 0.30 | 0.06 | 0.54 | 0.10 | 0.23 | 0.10 | 0.12 | 0.03 | 0.84 | 0.02 |
| Liver | 0.37 | 0.04 | 0.52 | 0.10 | 0.25 | 0.05 | 0.23 | 0.02 | 0.71 | 0.09 |
| Muscle | 0.20 | 0.06 | 0.54 | 0.05 | 0.04 | 0.02 | 0.07 | 0.01 | 0.69 | 0.14 |
| Tumor | 0.34 | 0.06 | 0.86 | 0.11 | 0.15 | 0.01 | 0.15 | 0.03 | 1.38 | 0.27 |

TABLE 15
Uptake in different organs and tissues obtained from dissections 1.5 h after
injection of ^{99m}Tc-labeled His₆-tagged VHHs in athymic nude mice bearing MDA-MB-231
tumors. Data expressed as % IA/g and presented as mean ± SD (n = 3).

| | ^{99m}Tc-B2 | | ^{99m}Tc-B1 | | ^{99m}Tc-R3B23 | | ^{99m}Tc-B3 | | ₉₉ₘTc-B4 | |
|---|---|---|---|---|---|---|---|---|---|---|
| Organ/tissue | Mean | SD | Mean | SD | Mean | SD | Mean | SD | Mean | SD |
| Blood | 0.41 | 0.06 | 0.83 | 0.19 | 0.46 | 0.22 | 0.24 | 0.03 | 1.38 | 0.13 |
| Lymph nodes | 0.78 | 0.54 | 3.98 | 1.46 | 0.32 | 0.11 | 0.26 | 0.03 | 2.76 | 1.71 |
| Heart | 0.24 | 0.03 | 0.42 | 0.10 | 0.20 | 0.07 | 0.12 | 0.01 | 0.60 | 0.05 |
| Lung | 0.49 | 0.08 | 0.58 | 0.09 | 0.43 | 0.12 | 0.29 | 0.03 | 0.79 | 0.09 |
| Liver | 0.59 | 0.04 | 0.69 | 0.05 | 0.41 | 0.08 | 0.49 | 0.05 | 0.82 | 0.11 |
| Pancreas | 0.16 | 0.02 | 0.64 | 0.23 | 0.14 | 0.05 | 0.08 | 0.00 | 0.94 | 0.10 |
| Spleen | 0.18 | 0.01 | 0.24 | 0.03 | 0.15 | 0.04 | 0.11 | 0.01 | 0.38 | 0.03 |
| Adrenals | 0.34 | 0.03 | 1.02 | 0.38 | 0.29 | 0.07 | 0.18 | 0.03 | 0.96 | 0.13 |
| Kidney | 191.74 | 2.74 | 169.55 | 25.44 | 219.80 | 3.07 | 249.34 | 26.52 | 36.96 | 0.64 |
| Stomach | 0.67 | 0.58 | 0.60 | 0.45 | 0.53 | 0.17 | 0.32 | 0.15 | 0.58 | 0.04 |
| Small intestine | 0.31 | 0.10 | 0.30 | 0.09 | 0.22 | 0.05 | 0.14 | 0.02 | 0.42 | 0.17 |
| Large intestine | 0.38 | 0.09 | 0.41 | 0.08 | 0.30 | 0.09 | 0.18 | 0.04 | 0.45 | 0.02 |
| Uterus | 0.77 | 0.26 | 1.40 | 0.49 | 0.32 | 0.05 | 0.22 | 0.16 | 1.88 | 0.50 |
| Skin | 0.63 | 0.09 | 1.91 | 0.72 | 0.36 | 0.10 | 0.26 | 0.02 | 2.49 | 0.20 |
| Muscle | 0.25 | 0.01 | 0.78 | 0.25 | 0.14 | 0.08 | 0.08 | 0.01 | 0.94 | 0.15 |
| Bone | 0.33 | 0.10 | 0.62 | 0.39 | 0.14 | 0.06 | 0.10 | 0.05 | 1.74 | 0.39 |
| Tumor | 0.57 | 0.11 | 0.99 | 0.47 | 0.31 | 0.01 | 0.36 | 0.07 | 1.71 | 0.27 |
| Brain | 0.02 | 0.01 | 0.04 | 0.01 | 0.03 | 0.01 | 0.02 | 0.01 | 0.07 | 0.03 |

### Example 6b

Secondly, untagged human/murine FAP-crossreactive VHH B1 was radiolabeled with ¹¹¹In, after which its in vitro and in vivo diagnostic potential was evaluated. The binding potential of the resulting radio-conjugate was assessed to confirm that this was not affected by the conjugation of ¹¹¹In-DOTA to the amino acid sequence of the VHH. To this, human FAP-expressing GM05389 cells were incubated with serial dilutions with concentrations ranging from 0 to 33 nM of the ¹¹¹In-labelled VHHs. A 100-fold excess of the corresponding unlabeled VHH was added in parallel to saturate the human FAP receptors expressed on cancer cells, to assess non-specific binding. Binding to human FAP receptor was not hampered, as an EC50 of 0.6±0.08 nM was obtained.

The in vivo diagnostic potential of untagged human/murine FAP-cross-reactive VHH B1, radiolabeled with ¹¹¹In, was assessed in athymic nude mice bearing HEK-293 tumors expressing human FAP receptor. To this, athymic nude mice (n=4 per time point) were inoculated subcutaneously with human FAP-expressing HEK-293 tumor cells in the neck. After validation of tumor growth (size about 100-200 mm³), all mice were intravenously injected in the tail vein with about 450 μCi (±7 μg) of ¹¹¹In-labeled VHH. Micro-SPECT/CT imaging was performed 1, 4 and 24 h after tracer injection using a MILabs VECTor⁺/CT system. In short, imaging was performed using a rat SPECT collimator and a spiral scan mode of 6 bed positions (3 min per position). For CT, a normal scan mode of only one position was used. The obtained Micro-SPECT/CT data are reconstructed with a 0.4 voxel size, 2 subsets and 4 iterations, after which images are fused and corrected for attenuation based on the CT scan. Images are analyzed using a medical image data analysis tool (AMIDE). Uptake values in organs and tissues were analyzed and expressed as % injected activity per cm³ (% IA/cc). Finally, mice were sacrificed after each scan and processed as described above. Organs and tissues of interest were weighed and measured for radioactivity using an automatic gamma counter, along with injection standards. Results were expressed as % of injected activity (IA)/g tissue.

Results indicated that ¹¹¹In-labeled VHH B1 accumulates specifically in tumors expressing human FAP. The uptake in tumor was consistent over time, with a value of about 2.52±0.16% IA/g and 1.01±0.18% IA/cc after 1 h and about 1.90±0.65% IA/g and 0.74±0.58% IA/cc after 24 h (Table 16 and 17). VHH B1 maintained its ability to bind murine FAP after radiolabeling with ¹¹¹In, with elevated retention of radioactivity in several organs and tissues such as blood, lymph nodes, bone, uterus, pancreas, skin and muscle that express and/or contain circulating murine FAP (in line with example 4f). This uptake in the abovementioned organs and tissues decreased over time and was close to background values at 24 h post injection.

TABLE 16
Uptake in different organs and tissues obtained from
SPECT/CT imaging 1, 4 and 24 h after injection of ¹¹¹In-labeled
untagged B1 in mice with human FAP-expressing HEK-
293 tumors. Data expressed as % IA/cc and presented
as mean ± SD (n = 3).

| | % IA/cc | | | | | |
|---|---|---|---|---|---|---|
| | 1 h pi | | 4 h pi | | 24 h pi | |
| Organ/tissue | Mean | SD | Mean | SD | Mean | SD |
| Tumor | 1.01 | 0.18 | 0.94 | 0.60 | 0.74 | 0.58 |
| kidney | 7.92 | 1.13 | 8.95 | 0.87 | 4.37 | 0.63 |
| Liver | 0.40 | 0.08 | 0.21 | 0.03 | 0.09 | 0.01 |
| Heart | 0.62 | 0.12 | 0.19 | 0.04 | 0.04 | 0.002 |
| Lung | 0.49 | 0.06 | 0.20 | 0.05 | 0.04 | 0.01 |
| Joint | 1.29 | 0.17 | 0.75 | 0.05 | 0.25 | 0.01 |
| Bone | 0.84 | 0.21 | 0.54 | 0.16 | 0.11 | 0.02 |
| Muscle | 0.42 | 0.16 | 0.33 | 0.07 | 0.07 | 0.01 |

TABLE 17
Uptake in different organs and tissues obtained from
dissections 1.5, 4.5 and 24.5 h after injection of ¹¹¹In-labeled
untagged B1 in mice with human FAP-expressing HEK-
293 tumors. Data expressed as % IA/g and presented
as mean ± SD (n = 3).

| | % IA/g | | | | | |
|---|---|---|---|---|---|---|
| | 1.5 h pi | | 4.5 h pi | | 24.5 h pi | |
| Organ/tissue | Mean | SD | Mean | SD | Mean | SD |
| Blood | 0.85 | 0.22 | 0.31 | 0.10 | 0.02 | 0.002 |
| Lymphnodes | 2.21 | 0.64 | 1.23 | 0.65 | 0.31 | 0.07 |
| Heart | 0.34 | 0.09 | 0.13 | 0.01 | 0.04 | 0.002 |
| Lung | 0.48 | 0.05 | 0.20 | 0.05 | 0.06 | 0.01 |
| Galbladder | 0.26 | 0.13 | 0.18 | 0.05 | 0.06 | 0.01 |
| Liver | 0.35 | 0.02 | 0.31 | 0.05 | 0.17 | 0.01 |
| Pancreas | 0.47 | 0.12 | 0.18 | 0.03 | 0.04 | 0.003 |
| Spleen | 0.20 | 0.04 | 0.12 | 0.02 | 0.07 | 0.004 |
| Adrenals | 0.64 | 0.12 | 0.33 | 0.01 | 0.09 | 0.01 |
| Kidney | 20.96 | 1.52 | 26.42 | 2.81 | 15.08 | 1.46 |
| Stomach | 0.43 | 0.25 | 0.15 | 0.02 | 0.04 | 0.003 |
| Small intestine | 0.23 | 0.04 | 0.12 | 0.02 | 0.04 | 0.004 |
| Large intestine | 0.33 | 0.06 | 0.23 | 0.11 | 0.05 | 0.004 |
| Ovary | 0.80 | 0.13 | 0.38 | 0.10 | 0.14 | 0.04 |
| Uterus | 1.57 | 0.28 | 0.74 | 0.13 | 0.53 | 0.20 |
| Skin | 1.77 | 0.15 | 1.14 | 0.12 | 0.34 | 0.02 |
| Muscle | 0.67 | 0.06 | 0.50 | 0.14 | 0.11 | 0.01 |
| Bone | 1.42 | 0.45 | 0.72 | 0.25 | 0.31 | 0.01 |
| Joint | 2.32 | 0.45 | 1.39 | 0.18 | 0.58 | 0.08 |
| Tumor | 2.52 | 0.16 | 2.04 | 0.57 | 1.90 | 0.65 |
| Brain | 0.04 | 0.01 | 0.02 | 0.01 | 0.004 | 0.001 |

### Example 7: Theranostic Use of the VHH Labelled with ¹³¹I

Theranostic use refers to the ability of one and the same labelled compound to be applicable for both diagnostic and therapeutic applications. In this example we describe the theranostic potential of human/murine FAP-cross-reactive VHH B1, both in its His₆-tagged version and its untagged version, human/murine FAP-cross-reactive VHH B2 and murine FAP-specific VHH B4 by investigating their targeting potential after radiolabelling with the theranostic radionuclide ¹³¹I (as described in example 3). Theranostic potential is evaluated in vitro by means of their ability to bind human FAP-expressing cells (saturation binding and cellular retention) and in vivo by assessing their biodistribution in a relevant mouse model.

### Example 7a

In a first part, the in vitro behaviour was assessed by means of investigating their cellular binding and retention overtime. The binding potential of the resulting radioconjugates was assessed to confirm that this was not affected by the introduction of ¹³¹I-SGMIB into the amino acid sequence of the VHH. To this, human FAP-expressing GM05389 (His₆-tagged and untagged VHH B1) and human FAP-transfected HEK-293 cells (His₆-tagged and untagged VHH B1; His₆-tagged VHH B2) were incubated with serial dilutions with concentrations ranging from 0 to 33 nM of the different ¹³¹I-labelled VHHs. A 100-fold excess of the corresponding unlabeled VHH was added in parallel to saturate the human FAP receptors expressed on cancer cells, to assess non-specific binding.

All ¹³¹I-labeled VHHs showed comparable dose-response curves in cell-binding experiments on human FAP-expressing GM05389 and HEK-293 cells, indicating that ¹³¹I-labeling did not affect binding potential. In the case of GM05389 cells, EC50 values of 0.6±0.1 and 2.7±0.1 nM were obtained for His₆-tagged B1 and untagged B1 respectively, while on HEK-293, EC50 values of 1.4-0.5, 2.8-0.7, and 3.8±3.3. nM were observed for His₆-tagged B1, untagged B1 and His₆-tagged B2 respectively.

The in vitro cellular retention of ¹³¹I-labeled His₆-tagged B1 and untagged B1 was assessed on human FAP-expressing GM05389 cells. In this particular case, cells were incubated with 10 nM ¹³¹I-labelled VHHs for 1 h at 4° C., after which the unbound fraction was collected. A 100-fold excess of the corresponding unlabeled VHH was added to assess non-specific binding. Next, the cells were incubated with fresh medium up to 24 h at 37° C., after which the dissociated fraction was collected. Afterwards, the cells were washed with 0.05 M glycine pH 2.8 to collect the membrane-bound fraction. Finally, cells were solubilized with 1 M NaOH at room temperature to collect the internalized fraction. The sum of the membrane-bound and internalized fractions corresponds to the total cell-associated fraction.

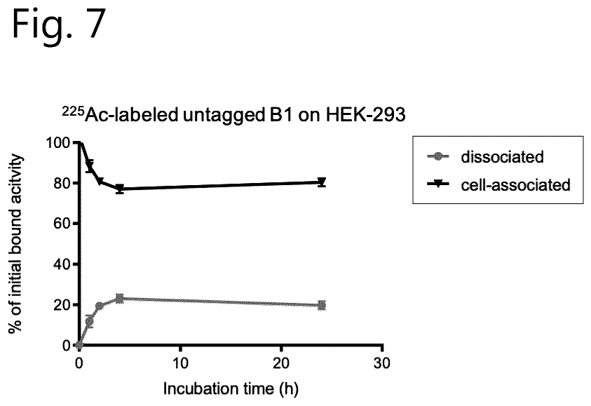

Both His₆-tagged B1 and untagged B1 reveal a very high level of cell-associated activity overtime upon human FAP-receptor binding. After 24 h incubation, about 80% and 70% of initial bound activity (for untagged and His₆-tagged B1 respectively) was still retained on tumor cells, reflecting the extensive and maintained targeting capacity of cross-reactive human/murine FAP-targeting VHH B1 (FIG. 5). This feature is very important for therapeutic applications using VHHs, as it allows for a prolonged exposure of target cells towards its cytotoxic payload.

### Example 7b

Next, the theranostic potential of ¹³¹I-labeled B1, B2 and B4 were assessed in mice with human FAP-expressing HEK-293 tumors. To this, athymic nude mice (n=3 per time point) were inoculated subcutaneously with human FAP-expressing HEK-293 tumor cells in the neck. After validation of tumor growth (size about 100-200 mm³), all mice were intravenously injected in the tail vein with about 400 μCi (±25 μg) of ¹³¹I-labeled VHHs. Micro-SPECT/CT imaging was performed 2 h after tracer injection using a MILabs VECTor⁺/CT system and according to the procedure described in D'Huyvetter et al. Clin Cancer Res. 2017 Nov. 1; 23(21):6616-6628. In short, imaging was performed using a mouse PET collimator and a spiral scan mode of 94 bed positions (19 s per position). For CT, a normal scan mode of only one position was used. The obtained Micro-SPECT/CT data are reconstructed with a 0.6 voxel size, 2 subsets and 7 iterations, after which images are fused and corrected for attenuation based on the CT scan. Images are analyzed using a medical image data analysis tool (AMIDE). Uptake values in organs and tissues were analyzed and expressed as % injected activity per cm³ (% IA/cc). Finally, mice were sacrificed after 2.5 h and processed as described above. Organs and tissues of interest were weighed and measured for radioactivity using an automatic gamma counter, along with injection standards. Results were expressed as % of injected activity (IA)/g tissue.

Results revealed specific accumulation of ¹³¹I-labeled B2 and B1 in human FAP-expressing HEK-293 tumors, while much lower uptake was observed for B4 (Table 18 and 19). Tumor uptake measured 2.96±0.47, 3.47±0.24, 1.15±0.44% IA/g for B2, B1, and B4, respectively. Uptake of ¹³¹I-labeled B1 in all additional organs and tissues such as lymph nodes, uterus and bone was in line with, however lower in absolute numbers, what has been seen for its ¹¹¹In-labeled variant (example 6b) and lower to what is observed for B4. Retention in kidneys after 2.5 h was low for all radioconjugates, with only 7.95±1.62, 4.77±1.01, 18.35±3.07% IA/g for B2, B1, and B4 respectively. The image quantification gave rise to similar results. This calculates to a therapeutic index (tumor-to-kidney ratio) of 0.45±0.08; 0.65±0.21, and 0.06±0.02 for B2, B1, and B4 respectively obtained from necropsy study, and ratios of 0.6±0.03, 0.9±0.28, and 0.13±0.04 respectively from imaging quantification. The therapeutic index for B2 and B1 was significantly higher compared to that obtained for B4, as indicated by one-way ANOVA (p<0.05). The therapeutic index of ¹³¹I-labeled B1 was higher, though not significantly, compared to that obtained for ¹³¹I-labeled B2.

TABLE 18
Uptake in different organs and tissues obtained from SPECT/CT
imaging 2 h after injection of ¹³¹I-labeled His -tagged B2,
B1, and B4 in mice with human FAP-expressing HEK-293 tumors.
Data expressed as % IA/cc and presented as mean ± SD (n = 3).

| | ¹³¹I-B2 | | ¹³¹I-B1 | | ¹³¹I-B4 | |
|---|---|---|---|---|---|---|
| Organ/tissue | Mean | SD | Mean | SD | MEAN | SD |
| Kidney | 3.41 | 0.22 | 2.34 | 0.30 | 4.66 | 1.04 |
| Heart | 0.14 | 0.02 | 0.32 | 0.02 | 0.42 | 0.02 |
| Lung | 0.18 | 0.00 | 0.32 | 0.00 | 0.42 | 0.06 |
| Liver | 0.92 | 0.10 | 0.75 | 0.26 | 0.44 | 0.08 |
| Muscle | 0.20 | 0.05 | 0.35 | 0.09 | 0.43 | 0.08 |
| Tumor | 2.05 | 0.21 | 1.98 | 0.54 | 0.61 | 0.26 |
| Tumor-to-kidney ratio | 0.6 | 0.03 | 0.9 | 0.28 | 0.13 | 0.04 |

TABLE 19
Uptake in different organs and tissues obtained from
dissections 2.5 h after injection of ¹³¹I-labeled
His6-tagged B2, B1, and B4 in mice with human FAP-
expressing HEK-293 tumors. Data expressed as % IA/g
and presented as mean ± SD (n = 3).

| | ¹³¹I-B2 | | ¹³¹I-B1 | | ¹³¹I-B4 | |
|---|---|---|---|---|---|---|
| Organ/tissue | Mean | SD | Mean | SD | Mean | SD |
| Blood | 0.13 | 0.03 | 0.28 | 0.02 | 0.75 | 0.14 |
| Lymph nodes | 0.52 | 0.21 | 1.74 | 0.29 | 1.86 | 0.81 |
| Thyroid | 0.45 | 0.15 | 1.12 | 0.36 | 2.18 | 1.13 |
| Heart | 0.16 | 0.02 | 0.20 | 0.01 | 0.41 | 0.05 |
| Lung | 0.27 | 0.03 | 0.26 | 0.04 | 0.68 | 0.14 |
| Galbladder | 0.45 | 0.15 | 0.45 | 0.32 | 0.56 | 0.19 |
| Liver | 1.68 | 0.21 | 0.97 | 0.28 | 0.66 | 0.21 |
| Pancreas | 0.40 | 0.04 | 0.45 | 0.02 | 0.81 | 0.28 |
| Spleen | 0.41 | 0.05 | 0.36 | 0.16 | 0.42 | 0.10 |
| Adrenals | 0.24 | 0.02 | 0.42 | 0.06 | 0.88 | 0.12 |
| Kidney | 7.95 | 1.62 | 4.77 | 1.01 | 18.35 | 3.07 |
| Stomach | 0.26 | 0.10 | 0.20 | 0.01 | 0.53 | 0.21 |
| Small intestine | 0.31 | 0.20 | 0.18 | 0.02 | 0.32 | 0.06 |
| Large intestine | 0.18 | 0.01 | 0.19 | 0.04 | 0.34 | 0.01 |
| Uterus | 0.31 | 0.05 | 0.67 | 0.19 | 2.47 | 1.98 |
| Skin | 0.55 | 0.24 | 0.79 | 0.02 | 2.05 | 0.35 |
| Muscle | 0.25 | 0.09 | 0.41 | 0.00 | 0.71 | 0.10 |
| Bone | 0.28 | 0.15 | 0.70 | 0.14 | 1.55 | 0.57 |
| Tumor | 3.47 | 0.24 | 2.96 | 0.47 | 1.15 | 0.44 |
| Brain | 0.03 | 0.02 | 0.03 | 0.01 | 0.04 | 0.01 |
| Tumor-to-kidney ratio | 0.45 | 0.08 | 0.65 | 0.21 | 0.06 | 0.02 |

### Example 7c

Next, the long-term biodistribution and tumor targeting potential of ¹³¹I-labeled untagged VHH B1 was evaluated in mice with human FAP-expressing HEK-293 tumors over 5 days post i.v. injection. To this, athymic nude mice (n=3 per time point) were inoculated subcutaneously with human FAP-expressing HEK-293 tumor cells in the neck. After validation of tumor growth (size of 315.98±346.80 mm³), all mice were intravenously injected in the tail vein with about 25 μCi (±5 μg) of ¹³¹I-labeled untagged VHH B1. Next, the mice were euthanized by cervical dislocation up to 120 h post injection, dissected after which different organs and tissues were collected. Organs and tissues of interest were weighed and measured for radioactivity using an automatic gamma counter, along with injection standards. Results were expressed as % of injected activity (IA)/g tissue.

Intravenous injection of ¹³¹I-labeled untagged VHH B1 reveals slightly elevated but transient uptake (about 2-3% IA/g, but decreasing over time) in organs and tissues such as lymph nodes, uterus, bone and skin (Table 20). The amount of activity in kidneys was relevant after two hours, with a value of 13.14±3.38% IA/g, however rapidly decreasing to <0.5% IA/g after 24 h. Uptake in tumor was significant, with a value of 2.79±1.37% IA/g after 2 h, surpassing the uptake in kidneys after 24 h with a value in tumor of 0.97±0.41% IA/g. Uptake in all other organs and tissues was low at all time points.

TABLE 20
uptake values in different organs and tissues for ¹³¹I-labeled untagged VHH B1 up to
120 h post i.v. injection, expressed as % injected activity per gram of tissue (% IA/g),
except for thyroid for which % IA is used. Data presented as mean ± SD (n = 3).

| | 2 h | | 4 h | | 24 h | | 48 h | | 72 h | | 96 h | | 120 h | |
|---|---|---|---|---|---|---|---|---|---|---|---|---|---|---|
| Organ/tissue | Mean | SD | Mean | SD | Mean | SD | Mean | SD | Mean | SD | Mean | SD | Mean | SD |
| Blood | 0.63 | 0.06 | 0.40 | 0.05 | 0.04 | 0.00 | 0.01 | 0.01 | 0.01 | 0.00 | 0.00 | 0.00 | 0.01 | 0.02 |
| Lymph nodes | 2.86 | 1.16 | 1.56 | 0.65 | 0.18 | 0.10 | 0.03 | 0.01 | 0.02 | 0.01 | 0.02 | 0.01 | 0.01 | 0.01 |
| Thyroid | 0.04 | 0.04 | 0.04 | 0.02 | 0.11 | 0.04 | 0.01 | 0.02 | 0.07 | 0.03 | 0.07 | 0.02 | 0.05 | 0.05 |
| Heart | 0.32 | 0.02 | 0.26 | 0.04 | 0.03 | 0.00 | 0.01 | 0.00 | 0.01 | 0.00 | 0.00 | 0.00 | 0.02 | 0.03 |
| Lung | 0.49 | 0.01 | 0.36 | 0.03 | 0.04 | 0.00 | 0.01 | 0.01 | 0.01 | 0.00 | 0.01 | 0.00 | 0.05 | 0.08 |
| Liver | 0.87 | 0.11 | 0.60 | 0.04 | 0.14 | 0.02 | 0.07 | 0.01 | 0.06 | 0.00 | 0.05 | 0.01 | 0.06 | 0.02 |
| Pancreas | 0.81 | 0.09 | 0.50 | 0.03 | 0.06 | 0.01 | 0.01 | 0.01 | 0.01 | 0.00 | 0.00 | 0.00 | 0.01 | 0.02 |
| Spleen | 0.28 | 0.03 | 0.24 | 0.03 | 0.06 | 0.02 | 0.03 | 0.01 | 0.02 | 0.00 | 0.02 | 0.01 | 0.04 | 0.03 |
| Kidney | 13.14 | 3.38 | 5.15 | 0.72 | 0.47 | 0.04 | 0.19 | 0.01 | 0.14 | 0.05 | 0.08 | 0.01 | 0.07 | 0.03 |
| Stomach | 0.55 | 0.04 | 0.33 | 0.04 | 0.05 | 0.01 | 0.03 | 0.02 | 0.02 | 0.01 | 0.01 | 0.00 | 0.03 | 0.03 |
| Small intestine | 0.50 | 0.09 | 0.23 | 0.05 | 0.03 | 0.01 | 0.05 | 0.06 | 0.01 | 0.00 | 0.01 | 0.00 | 0.03 | 0.03 |
| Large intestine | 0.38 | 0.07 | 0.26 | 0.07 | 0.03 | 0.01 | 0.01 | 0.01 | 0.01 | 0.00 | 0.01 | 0.00 | 0.14 | 0.13 |
| Uterus | 1.70 | 0.39 | 0.95 | 0.23 | 0.20 | 0.12 | 0.05 | 0.03 | 0.02 | 0.01 | 0.01 | 0.00 | 0.23 | 0.31 |
| Skin | 2.32 | 0.25 | 1.83 | 0.31 | 0.40 | 0.13 | 0.06 | 0.01 | 0.02 | 0.01 | 0.01 | 0.00 | 0.04 | 0.04 |
| Muscle | 0.68 | 0.20 | 0.67 | 0.08 | 0.07 | 0.02 | 0.02 | 0.01 | 0.01 | 0.00 | 0.00 | 0.00 | 0.07 | 0.07 |
| Bone | 1.52 | 0.24 | 1.07 | 0.11 | 0.18 | 0.02 | 0.03 | 0.01 | 0.02 | 0.01 | 0.01 | 0.00 | 0.18 | 0.20 |
| Tumor | 2.79 | 1.37 | 2.48 | 0.94 | 0.97 | 0.41 | 0.31 | 0.22 | 0.21 | 0.23 | 0.11 | 0.12 | 0.16 | 0.08 |
| Brain | 0.07 | 0.02 | 0.03 | 0.01 | 0.01 | 0.00 | 0.00 | 0.00 | 0.00 | 0.00 | 0.00 | 0.00 | 0.01 | 0.02 |

Taken together, this example indicates that targeting of human FAP-expression in vitro on cells and in vivo in tumors is feasible with ¹³¹I-labeled cross-reactive human/murine FAP-targeting VHH B1, both in its His₆-tagged (SEQ ID NO: 21) and untagged format (SEQ ID NO: 4). The in vivo tumor targeting potential combined with low retention of radioactivity in additional organs and tissues supports its therapeutic application.

### Example 8: Theranostic Use of the VHH Labelled with ¹⁷⁷Lu

Theranostic use refers to the ability of one and the same labelled compound to be applicable for both diagnostic and therapeutic applications. In this example we describe the theranostic potential of cross-reactive human/murine FAP-targeting, untagged VHH B1, by investigating its targeting potential after radiolabelling with the theranostic radionuclide ¹⁷⁷Lu (as described in example 3). Theranostic potential is evaluated in vitro by means of its ability to bind human FAP-expressing cells (saturation binding and cellular retention) and in vivo by assessing its biodistribution in a relevant mouse model.

In a first part, its in vitro behaviour was assessed by means of investigating its cellular binding and retention overtime. The binding potential of the resulting radioconjugates was assessed to confirm that this was not affected by the introduction of ¹⁷⁷Lu-DTPA into the amino acid sequence of the VHH. To this, human FAP-expressing GM05389 and HEK-293 cells were incubated with serial dilutions with concentrations ranging from 0 to 33 nM of ¹⁷⁷Lu-labeled untagged VHH B1. A 100-fold excess of the corresponding unlabeled VHH was added in parallel to saturate the human FAP receptors expressed on cancer cells, to assess non-specific binding.

On both cell lines, binding of ¹⁷⁷Lu-labeled untagged VHH B1 revealed comparable dose-response curves on human FAP-expressing GM05389 and HEK-293 cells, indicating that the introduction of ¹⁷⁷Lu-DTPA did not affect the binding potential. In the case of GM05389 cells, an EC50 value of 0.5±0.1 nM was obtained, while on HEK-293, an EC50 value of 0.8±0.1 nM was observed.

The in vitro cellular retention of ¹⁷⁷Lu-labeled untagged VHH B1 was assessed on human FAP-expressing GM05389 and HEK-293 cells. In this particular case, cells were incubated with 10 nM ¹⁷⁷Lu-labeled untagged B1 for 1 h at 4° C., after which the unbound fraction was collected. A 100-fold excess of the corresponding unlabeled VHH was used to assess non-specific binding. Next, the cells were incubated with fresh medium up to 24 h at 37° C., after which the dissociated fraction was collected. Afterwards, the cells were washed with 0.05 M glycine pH 2.8 to collect the membrane-bound fraction. Finally, cells were solubilized with 1 M NaOH at room temperature to collect the internalized fraction. The sum of the membrane-bound and internalized fractions corresponds to the total cell-associated fraction.

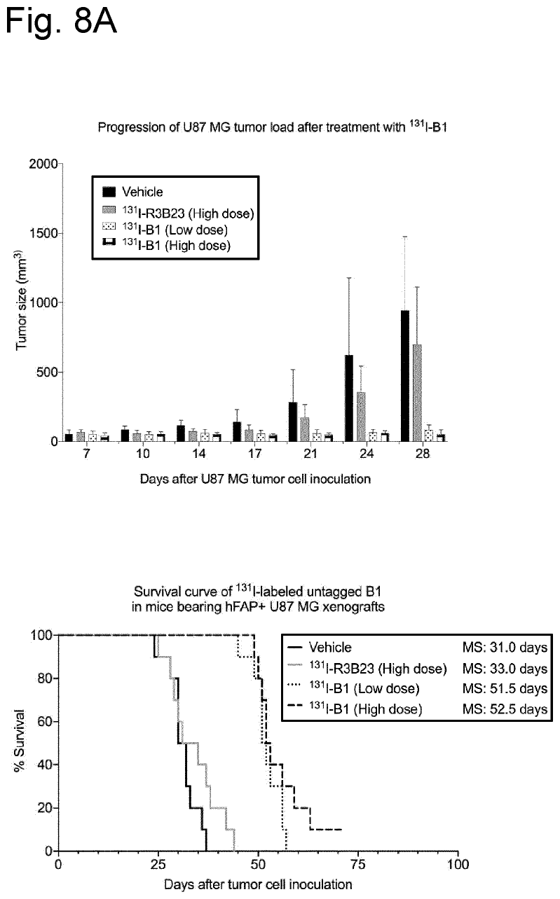

In both cases, untagged VHH B1 reveals a very high level of cell-associated activity over time upon human FAP-receptor binding. After 24 h incubation, about 80% and 65% of initial bound activity was still retained on HEK-293 and GM05389 cells, respectively, reflecting the extensive and maintained targeting capacity of cross-reactive human/murine FAP-targeting VHH B1 (FIG. 6). This feature is very important for therapeutic applications using VHHs, as it allows for a prolonged exposure of target cells towards its cytotoxic payload.

Next, the long-term biodistribution and tumor targeting potential of ¹⁷⁷Lu-labeled untagged VHH B1 was evaluated in mice with human FAP-expressing HEK-293 tumors over 5 days post i.v. injection. To this, athymic nude mice (n=3 per time point) were inoculated subcutaneously with human FAP-expressing HEK-293 tumor cells in the neck. After validation of tumor growth (size of 43.67±26.61 mm3), all mice were intravenously injected in the tail vein with about 80 μCi (±5 μg) of ¹⁷⁷Lu-labeled untagged VHH B1. Next, the mice were euthanized by cervical dislocation up to 120 h post injection, dissected after which different organs and tissues were collected. Organs and tissues of interest were weighed and measured for radioactivity using an automatic gamma counter, along with injection standards. Results were expressed as % of injected activity (IA)/g tissue.

Intravenous injection of ¹⁷⁷Lu-labeled untagged VHH B1 reveals slightly elevated but transient uptake (about 2-3% IA/g, but decreasing overtime) in organs and tissues such as adrenals, uterus, bone and skin (Tables 16 and 17). The amount of activity in kidneys was relevant after two hours, with a value of 94.05±9.58% IA/g, and in contrast to what has been observed for ¹³¹I-labeled VHH B1 not rapidly decreasing over time. Indeed the uptake in kidney was still 31.72±1.68% IA/g after 24 h. Uptake in tumor was significant, with a value of 3.90±0.35% IA/g after 2 h, and remained high over time, with still 2.15±0.98% IA/g after 24 h, which is higher compared to what is observed for ¹³¹I-labeled VHH B1. Uptake in all other organs and tissues was low at all time points.

TABLE 21
uptake values in different organs and tissues for ¹⁷⁷Lu-labeled
untagged VHH B1 up to 24 h post i.v. injection, expressed as %
injected activity per gram of tissue (% IA/g), except for lymph
nodes for which % IA is used. Data presented as mean ± SD (n = 3).

| | 1 h | | 2 h | | 6 h | | 24 h | |
|---|---|---|---|---|---|---|---|---|
| Organ/tissue | Mean | SD | Mean | SD | Mean | SD | Mean | SD |
| Blood | 0.80 | 0.14 | 0.69 | 0.03 | 0.21 | 0.03 | 0.01 | 0.01 |
| Lymph nodes | 0.02 | 0.01 | 0.02 | 0.01 | 0.01 | 0.00 | 0.00 | 0.00 |
| Heart | 0.35 | 0.03 | 0.33 | 0.03 | 0.14 | 0.02 | 0.05 | 0.01 |
| Lung | 0.62 | 0.03 | 0.71 | 0.16 | 0.30 | 0.04 | 0.09 | 0.02 |
| Galbladder | 0.59 | 0.29 | 0.77 | 0.93 | 0.31 | 0.23 | 0.18 | 0.08 |
| Liver | 0.38 | 0.03 | 0.48 | 0.03 | 0.47 | 0.04 | 0.28 | 0.01 |
| Pancreas | 0.62 | 0.06 | 0.55 | 0.02 | 0.26 | 0.07 | 0.07 | 0.01 |
| Spleen | 0.28 | 0.08 | 0.23 | 0.05 | 0.20 | 0.01 | 0.11 | 0.03 |
| Adrenals | 1.77 | 0.29 | 1.49 | 0.34 | 0.81 | 0.14 | 0.33 | 0.11 |
| Kidney | 81.65 | 4.86 | 94.05 | 9.58 | 75.68 | 6.29 | 31.72 | 1.68 |
| Stomach | 0.58 | 0.25 | 0.40 | 0.04 | 0.22 | 0.03 | 0.07 | 0.01 |
| Small intestine | 0.38 | 0.08 | 0.27 | 0.04 | 0.15 | 0.04 | 0.05 | 0.01 |
| Large intestine | 0.56 | 0.08 | 0.41 | 0.07 | 0.22 | 0.08 | 0.09 | 0.02 |
| Uterus | 1.73 | 0.18 | 2.26 | 0.32 | 1.04 | 0.51 | 0.26 | 0.07 |
| Skin | 2.25 | 0.37 | 2.19 | 0.23 | 1.39 | 0.20 | 0.39 | 0.06 |
| Muscle | 0.78 | 0.13 | 0.77 | 0.21 | 0.34 | 0.03 | 0.06 | 0.02 |
| Bone | 1.75 | 0.52 | 1.65 | 0.14 | 1.02 | 0.21 | 0.23 | 0.02 |
| Tumor | 3.68 | 0.60 | 3.90 | 0.35 | 5.31 | 0.12 | 2.15 | 0.98 |
| Brain | 0.04 | 0.01 | 0.03 | 0.01 | 0.02 | 0.01 | 0.02 | 0.01 |

TABLE 22
uptake values in different organs and tissues for ¹⁷⁷Lu-labeled
untagged VHH B1 from 48 h up to 120 h post i.v. injection,
expressed as % injected activity per gram of tissue (%
IA/g), except for lymph nodes for which % IA is used. Data
presented as mean ± SD (n = 3).

| | 48 h | | 72 h | | 96 h | | 120 h | |
|---|---|---|---|---|---|---|---|---|
| Organ/tissue | Mean | SD | Mean | SD | Mean | SD | Mean | SD |
| Blood | 0.00 | 0.00 | 0.00 | 0.00 | 0.00 | 0.00 | 0.00 | 0.00 |
| Lymph nodes | 0.00 | 0.00 | 0.00 | 0.00 | 0.00 | 0.00 | 0.00 | 0.00 |
| Heart | 0.05 | 0.01 | 0.03 | 0.01 | 0.02 | 0.01 | 0.01 | 0.01 |
| Lung | 0.07 | 0.01 | 0.05 | 0.01 | 0.03 | 0.02 | 0.01 | 0.01 |
| Galbladder | 0.04 | 0.04 | 0.21 | 0.30 | 0.03 | 0.05 | 0.03 | 0.04 |
| Liver | 0.20 | 0.02 | 0.21 | 0.04 | 0.15 | 0.01 | 0.11 | 0.03 |
| Pancreas | 0.03 | 0.01 | 0.04 | 0.01 | 0.02 | 0.00 | 0.01 | 0.01 |
| Spleen | 0.09 | 0.02 | 0.14 | 0.11 | 0.07 | 0.01 | 0.06 | 0.02 |
| Adrenals | 0.35 | 0.06 | 0.12 | 0.08 | 0.10 | 0.09 | 0.09 | 0.06 |
| Kidney | 23.12 | 4.84 | 11.86 | 2.02 | 6.68 | 0.78 | 4.56 | 1.04 |
| Stomach | 0.05 | 0.01 | 0.04 | 0.02 | 0.03 | 0.01 | 0.02 | 0.01 |
| Small intestine | 0.03 | 0.00 | 0.02 | 0.00 | 0.01 | 0.01 | 0.01 | 0.01 |
| Large intestine | 0.06 | 0.01 | 0.05 | 0.03 | 0.02 | 0.03 | 0.03 | 0.01 |
| Uterus | 0.13 | 0.01 | 0.11 | 0.04 | 0.08 | 0.04 | 0.11 | 0.05 |
| Skin | 0.25 | 0.04 | 0.16 | 0.04 | 0.14 | 0.01 | 0.12 | 0.03 |
| Muscle | 0.05 | 0.03 | 0.03 | 0.02 | 0.01 | 0.02 | 0.03 | 0.02 |
| Bone | 0.26 | 0.06 | 0.31 | 0.05 | 0.15 | 0.08 | 0.13 | 0.06 |
| Tumor | 1.54 | 0.37 | 1.16 | 0.38 | 0.48 | 0.20 | 0.44 | 0.15 |
| Brain | 0.00 | 0.00 | 0.00 | 0.00 | 0.00 | 0.00 | 0.00 | 0.00 |

A co-administration with 150 mg/kg gelofusin was able to reduce kidney retention of ¹⁷⁷Lu-labeled untagged VHH BM significantly, to an uptake value in kidney of only 15.58±1.46% IA/g, obtained already after 1 h (Table 23). Tumor accumulation remained high and specific, as the uptake of ¹⁷⁷Lu-labeled non-targeting control VHH R3B23 only measured 0.28±0.24% IA/g at the same time point.

TABLE 23
uptake values in different organs and tissues for ¹⁷⁷Lu-labeled
untagged VHH B1 and non-targeting control VHH R3B23 at 1 h
post i.v. injection, expressed as % injected activity per gram
of tissue (% IA/g). Data presented as mean ± SD (n = 3).

| | |
|---|---|
| ¹⁷⁷Lu-B1 | ¹⁷⁷Lu-R3B23 |

| Organ/tissue | Mean | SD | Mean | SD |
|---|---|---|---|---|
| Blood | 0.58 | 0.07 | 0.13 | 0.02 |
| Lymph nodes | 2.15 | 0.83 | 0.28 | 0.07 |
| Heart | 0.28 | 0.04 | 0.10 | 0.02 |
| Lung | 0.37 | 0.04 | 0.21 | 0.04 |
| Galbladder | 0.86 | 0.25 | 0.61 | 0.37 |
| Liver | 0.27 | 0.05 | 0.20 | 0.04 |
| Pancreas | 0.68 | 0.20 | 0.07 | 0.01 |
| Spleen | 0.17 | 0.04 | 0.09 | 0.01 |
| Adrenals | 0.50 | 0.05 | 0.10 | 0.00 |
| Kidney | 15.58 | 1.46 | 15.27 | 0.56 |
| Stomach | 0.37 | 0.14 | 0.16 | 0.02 |
| Small intestine | 0.26 | 0.07 | 0.11 | 0.02 |
| Large intestine | 0.24 | 0.02 | 0.17 | 0.03 |
| Uterus | 1.15 | 0.16 | 0.23 | 0.07 |
| Skin | 1.90 | 0.19 | 0.44 | 0.04 |
| Muscle | 0.61 | 0.12 | 0.10 | 0.01 |
| Bone | 1.21 | 0.33 | 0.07 | 0.01 |
| Tumor | 2.79 | 0.42 | 0.28 | 0.24 |
| Brain | 0.03 | 0.01 | 0.01 | 0.00 |

Taken together, this example indicates that targeting of human FAP-expression in vitro on cells and in vivo in tumors is feasible with ¹⁷⁷Lu-labeled cross-reactive human/murine FAP-targeting VHH B1. The in vivo tumor targeting potential combined with the fact that the uptake in kidneys can be reduced significantly (by co-injection with gelofusin) to a level comparable to what is observed for its ¹³¹I-SGMIB labelled variant supports its therapeutic application.

### Example 9: Therapeutic Use of the VHH Labelled with ²²⁵Ac

In this example we describe the therapeutic potential of cross-reactive human/murine FAP-targeting, untagged VHH B1, by investigating its targeting capacity after radiolabelling with the therapeutic radionuclide ²²⁵Ac (as described in example 3). Therapeutic potential is evaluated in vitro by means of its ability to bind human FAP-expressing cells (saturation binding and cellular retention) and in vivo by assessing its biodistribution in a relevant mouse model.

In a first part, its in vitro behaviour was assessed by means of investigating its cellular binding and retention over time. The binding potential of the resulting radioconjugates was assessed to confirm that this was not affected by the introduction of ²²⁵Ac-DOTA into the amino acid sequence of the VHH. To this, human FAP-expressing GM05389 cells were incubated with serial dilutions with concentrations ranging from 0 to 33 nM of ²²⁵Ac-labeled untagged VHH B1. A 100-fold excess of the corresponding unlabeled VHH was added in parallel to saturate the human FAP receptors expressed on cancer cells, to assess non-specific binding.

Binding of ²²⁵Ac-labeled untagged VHH B1 revealed a dose-response curve on human FAP-expressing GM05389 cells comparable to what was obtained for its ¹³¹I-SGMIB and ¹⁷⁷Lu-DTPA variant, indicating that the introduction of ²²⁵Ac-DOTA did not affect binding potential. An EC50 value of 0.4±0.1 nM was obtained.

The in vitro cellular retention of ²²⁵Ac-labeled untagged VHH B1 was assessed on human FAP-expressing GM05389 cells. In this particular case, cells were incubated with 10 nM ²²⁵Ac-labeled untagged VHH B1 for 1 h at 4° C., after which the unbound fraction was collected. A 100-fold excess of the corresponding unlabeled VHH was used to assess non-specific binding. Next, the cells were incubated with fresh medium up to 24 h at 37° C., after which the dissociated fraction was collected. Afterwards, the cells were washed with 0.05 M glycine pH 2.8 to collect the membrane-bound fraction. Finally, cells were solubilized with 1 M NaOH at room temperature to collect the internalized fraction. The sum of the membrane-bound and internalized fractions corresponds to the total cell-associated fraction.

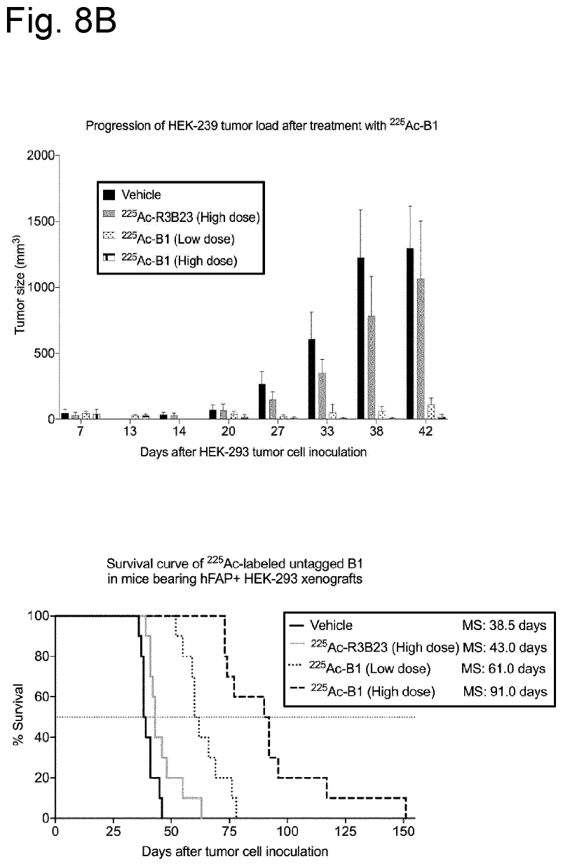

²²⁵Ac-labeled untagged VHH B1 reveals a very high level of cell-associated activity over time upon human FAP-receptor binding. After 24 h incubation, about 80% of initial bound activity was still retained on GM05389 cells reflecting the extensive and maintained targeting capacity of cross-reactive human/murine FAP-targeting VHH B1 (FIG. 7). This feature is very important for therapeutic applications using VHHs, as it allows for a prolonged exposure of target cells towards its cytotoxic payload.

Next, the long-term biodistribution and tumor targeting potential of ²²⁵Ac-labeled untagged VHH B1 was evaluated in mice with human FAP-expressing HEK-293 tumors over 4 days post i.v. injection. To this, athymic nude mice (n=3 per time point) were inoculated subcutaneously with human FAP-expressing HEK-293 tumor cells in the neck. After validation of tumor growth (size of 60.71±39.10 mm3), all mice were intravenously injected in the tail vein with about 1.6 μCi (±5 μg) of ²²⁵Ac-labeled untagged VHH B1, co-injected with 150 mg/kg gelofusin. Next, the mice were euthanized by cervical dislocation up to 96 h post injection, dissected after which different organs and tissues were collected. Organs and tissues of interest were weighed and measured for radioactivity using an automatic gamma counter, along with injection standards. Results were expressed as % of injected activity (IA)/g tissue.

Intravenous injection of ²²⁵Ac-labeled untagged VHH B1 reveals slightly elevated but transient uptake (about 2-4% IA/g, but decreasing over time) in organs and tissues such as intestines, uterus and skin. The uptake in bone ranged around 5% IA/g at all time points (Table 24). The amount of activity in kidneys was relevant after 1 h, with a value of 13.48±1.58% IA/g, and decreased to about 6.35±1.30% IA/g after 96 h. Uptake in tumor was significant, with a value of 3.83±0.53% IA/g after 1 h, and remained high overtime, with still 3.12±0.62% IA/g after 24 h, and 2.54±2.07% IA/g after 96 h, which is higher compared to what is observed for ¹³¹I-labeled and ¹⁷⁷Lu-labeled untagged VHH B1. Uptake in all other organs and tissues was low at all time points.

TABLE 24
uptake values in different organs and tissues for ²²⁵Ac-labeled
untagged VHH B1 up to 96 h post i.v. injection, expressed as %
injected activity per gram of tissue (% IA/g), except for lymph
nodes for which % IA is used. Data presented as mean ± SD (n = 3).

| | 1 h | | 4 h | | 24 h | | 72 h | | 96 h | |
|---|---|---|---|---|---|---|---|---|---|---|
| Organ | Mean | SD | Mean | SD | Mean | SD | Mean | SD | Mean | SD |
| Blood | 1.14 | 0.53 | 1.02 | 0.46 | 1.27 | 0.97 | 0.57 | 0.33 | 0.97 | 0.38 |
| Lymph nodes | 0.09 | 0.02 | 0.08 | 0.01 | 0.07 | 0.02 | 0.09 | 0.00 | 0.10 | 0.03 |
| Heart | 1.10 | 0.20 | 0.94 | 0.13 | 0.72 | 0.11 | 0.70 | 0.19 | 0.90 | 0.09 |
| Lung | 1.72 | 1.29 | 1.56 | 0.86 | 0.94 | 0.21 | 4.31 | 2.13 | 1.91 | 0.11 |
| Liver | 1.65 | 0.20 | 2.08 | 0.20 | 2.18 | 0.44 | 2.00 | 0.11 | 2.12 | 0.57 |
| Pancreas | 1.07 | 0.15 | 0.59 | 0.05 | 0.48 | 0.10 | 0.51 | 0.08 | 0.67 | 0.18 |
| Spleen | 0.93 | 0.22 | 0.68 | 0.05 | 0.74 | 0.18 | 0.70 | 0.06 | 0.79 | 0.17 |
| Kidney | 13.48 | 1.58 | 17.94 | 1.02 | 13.74 | 2.56 | 8.49 | 0.79 | 6.35 | 1.30 |
| Stomach | 0.83 | 0.17 | 0.82 | 0.18 | 0.76 | 0.22 | 0.56 | 0.07 | 0.71 | 0.14 |
| Small intestine | 1.16 | 0.37 | 0.74 | 0.02 | 0.75 | 0.26 | 0.62 | 0.22 | 0.64 | 0.16 |
| Large intestine | 2.47 | 0.68 | 1.67 | 0.36 | 2.27 | 0.89 | 1.89 | 0.23 | 3.05 | 1.31 |
| Uterus | 2.60 | 1.02 | 2.74 | 0.69 | 2.01 | 0.69 | 2.70 | 0.56 | 1.91 | 0.47 |
| Skin | 2.06 | 0.43 | 1.53 | 0.22 | 1.03 | 0.54 | 1.11 | 0.16 | 1.51 | 0.57 |
| Muscle | 1.29 | 0.28 | 1.16 | 0.17 | 0.85 | 0.29 | 1.05 | 0.33 | 1.15 | 0.16 |
| Bone | 4.10 | 0.41 | 4.10 | 0.74 | 3.12 | 0.62 | 6.71 | 3.05 | 7.05 | 3.53 |
| Tumor | 3.83 | 0.53 | 4.17 | 0.77 | 4.25 | 1.10 | 2.51 | 1.10 | 2.54 | 2.07 |
| Brain | 0.24 | 0.05 | 0.19 | 0.03 | 0.25 | 0.06 | 0.25 | 0.06 | 0.37 | 0.14 |

### Example 10: Biodistribution of Radiolabeled Variants of Untagged VHH B1

In addition to the described results in example 7, 8 and 9, the long-term biodistribution and tumor targeting potential of ¹³¹I-labeled; ²²⁵Ac-labeled and ¹⁷⁷Lu-labeled untagged VHH B1 was evaluated over 4 days post i.v. injection in mice with human glioblastoma tumors (U87 MG) that naturally express human FAP. To this, athymic nude mice (n=3 per time point) were inoculated subcutaneously with human FAP-expressing HEK-293 tumor cells in the neck. After validation of tumor growth (150-250 mm³), mice were intravenously injected in the tail vein with about 80 μCi (±5 μg) of ¹³¹I-labeled; 0.5 μCi (±5 μg) ²²⁵Ac-labeled or 100 μCi (±5 μg) ¹⁷⁷Lu-labeled untagged VHH B1. Next, the mice were euthanized by cervical dislocation up to 96 h post injection, dissected after which different organs and tissues were collected. Organs and tissues of interest were weighed and measured for radioactivity using an automatic gamma counter, along with injection standards. Results were expressed as % of injected activity (IA)/g tissue.

Intravenous injection of ¹³¹I-labeled untagged VHH B1 reveals slightly elevated but transient uptake (about 2-3% IA/g, but decreasing over time) in organs and tissues such as lymph nodes, uterus, bone and skin (Table 1). The amount of activity in kidneys was relevant after three hours, with a value of 8.64-0.56% IA/g, however rapidly decreasing to <1% IA/g after 24 h. Uptake in tumor was significant, with a value of 10.89±3.81% IA/g after 3 h, surpassing the uptake in kidneys already after 3 h. Uptake in all other organs and tissues was low at all time points.

the administration of ²²⁵Ac-labeled untagged VHH B1 reveals slightly elevated but transient uptake (about 2-3% IA/g, but decreasing over time) in organs and tissues such as lymph nodes, uterus, bone and skin (Table 2). The amount of activity in kidneys was relevant after three hours, with a value of 12.30-0.53% IA/g, slowly decreasing to 8.07±1.39% IA/g after 24 h and to 2.47±0.18% IA/g after 96 h. Uptake in tumor was significant, with a value of 5.03±1.74% IA/g after 3 h, however never surpassing the uptake in kidneys. Uptake in all other organs and tissues was low at all time points.

The administration of ¹⁷⁷Lu-labeled untagged VHH B1 reveals slightly elevated but transient uptake (about 2-3% IA/g, but decreasing over time) in organs and tissues such as lymph nodes, uterus, bone and skin (Table 3). The amount of activity in kidneys was relevant after four hours, with a value of 42.27±1.58% IA/g, slowly decreasing to 28.38±2.02% IA/g after 24 h and to 5.02±0.53% IA/g after 96 h. Uptake in tumor was significant, with a value of 10.52±2.25% IA/g after 4 h, however never surpassing the uptake in kidneys. Uptake in all other organs and tissues was low at all time points. Importantly, the uptake in kidneys for ¹⁷⁷Lu-labeled untagged VHH B1 was significant higher than the values obtained for the ²²⁵Ac-labeled variant at all investigated time points.

From the uptake values, the corresponding tumor-to-kidney (T/K) ratios over time were calculated for each of the radiolabeled variants of untagged VHH B1. For the ¹³¹I-labeled variant of untagged VHH B1, T/K>1 were obtained as of 3 h post administration onwards, with a peak value of 11.13±3.02 at 48 h post injection. For both ²²⁵Ac-labeled and ¹⁷⁷Lu-labeled untagged VHH B1 no T/K ratios >1 were obtained, however, for ²²⁵Ac-labeled untagged VHH B1, a peak T/K ratio of 0.79±0.19 at 48 h was calculated, while for the ¹⁷⁷Lu-labeled variant the peak T/K only measured 0.37±0.05 after 72 h.

TABLE 25
uptake values in different organs and tissues for ¹³¹I-labeled untagged VHH B1 up to 96 h post i.v. injection, expressed as %
injected activity per gram of tissue (% IA/g), except for thyroid for which % IA is used. Data presented as mean ± SD (n = 3).

| | 1 h | | 3 h | | 6 h | | 24 h | | 48 h | | 72 h | | 96 h | |
|---|---|---|---|---|---|---|---|---|---|---|---|---|---|---|
| Organ/tissue | MEAN | SD | MEAN | SD | MEAN | SD | MEAN | SD | MEAN | SD | MEAN | SD | MEAN | SD |
| Blood | 1.48 | 0.50 | 0.51 | 0.02 | 0.34 | 0.15 | 0.03 | 0.01 | 0.01 | 0.00 | 0.01 | 0.00 | 0.00 | 0.00 |
| Lymphnodes | 3.19 | 0.63 | 1.32 | 0.20 | 1.22 | 0.38 | 0.11 | 0.04 | 0.03 | 0.02 | 0.07 | 0.01 | 0.02 | 0.01 |
| Thyroid (% IA) | 0.07 | 0.02 | 0.05 | 0.04 | 0.03 | 0.04 | 0.11 | 0.08 | 0.11 | 0.01 | 0.24 | 0.05 | 0.10 | 0.03 |
| Thymus | 0.05 | 0.05 | 0.51 | 0.11 | 0.23 | 0.03 | 0.07 | 0.01 | 0.02 | 0.01 | 0.05 | 0.06 | 0.01 | 0.01 |
| Heart | 0.68 | 0.13 | 0.27 | 0.01 | 0.20 | 0.06 | 0.03 | 0.01 | 0.01 | 0.00 | 0.02 | 0.01 | 0.00 | 0.01 |
| Lung | 0.98 | 0.13 | 0.53 | 0.06 | 0.28 | 0.09 | 0.04 | 0.01 | 0.03 | 0.01 | 0.03 | 0.01 | 0.01 | 0.01 |
| Liver | 1.12 | 0.10 | 0.73 | 0.04 | 1.04 | 1.03 | 0.16 | 0.02 | 0.13 | 0.01 | 0.22 | 0.01 | 0.09 | 0.01 |
| Pancreas | 1.27 | 0.11 | 0.47 | 0.12 | 0.38 | 0.18 | 0.04 | 0.01 | 0.01 | 0.00 | 0.01 | 0.00 | 0.00 | 0.00 |
| Spleen | 0.62 | 0.08 | 0.85 | 0.74 | 0.34 | 0.14 | 0.09 | 0.02 | 0.05 | 0.01 | 0.16 | 0.07 | 0.04 | 0.02 |
| Adrenals | 1.85 | 0.02 | 0.58 | 0.40 | 0.67 | 0.25 | 0.07 | 0.03 | 0.03 | 0.01 | 0.03 | 0.00 | 0.03 | 0.01 |
| Kidney | 43.97 | 3.61 | 8.64 | 0.56 | 4.68 | 0.82 | 0.73 | 0.18 | 0.41 | 0.01 | 0.62 | 0.21 | 0.16 | 0.02 |
| Stomach | 0.79 | 0.01 | 0.66 | 0.35 | 0.32 | 0.17 | 0.03 | 0.01 | 0.02 | 0.01 | 0.02 | 0.01 | 0.02 | 0.01 |
| Small intestine | 0.70 | 0.10 | 0.26 | 0.07 | 0.24 | 0.15 | 0.03 | 0.02 | 0.01 | 0.00 | 0.01 | 0.01 | 0.01 | 0.01 |
| Large intestine | 1.31 | 0.19 | 0.37 | 0.04 | 0.29 | 0.16 | 0.02 | 0.01 | 0.01 | 0.00 | 0.03 | 0.02 | 0.02 | 0.02 |
| Uterus | 2.39 | 0.35 | 1.35 | 0.22 | 0.60 | 0.03 | 0.13 | 0.04 | 0.02 | 0.01 | 0.02 | 0.00 | 0.01 | 0.01 |
| Ovary | 0.98 | 0.90 | 0.26 | 0.23 | 0.64 | 0.57 | 0.05 | 0.01 | 0.01 | 0.01 | 0.02 | 0.00 | 0.00 | 0.01 |
| Skin | 3.53 | 0.29 | 1.92 | 0.03 | 1.35 | 0.30 | 0.14 | 0.08 | 0.03 | 0.01 | 0.03 | 0.01 | 0.01 | 0.01 |
| Muscle | 1.21 | 0.18 | 0.62 | 0.14 | 0.53 | 0.09 | 0.02 | 0.02 | 0.02 | 0.00 | 0.04 | 0.05 | 0.00 | 0.00 |
| Bone | 2.59 | 0.29 | 1.43 | 0.47 | 1.16 | 0.27 | 0.13 | 0.03 | 0.04 | 0.01 | 0.03 | 0.01 | 0.01 | 0.01 |
| Joint | 4.89 | 1.31 | 2.20 | 0.49 | 1.80 | 0.47 | 0.25 | 0.05 | 0.05 | 0.02 | 0.04 | 0.02 | 0.01 | 0.00 |
| Tumor | 15.61 | 2.49 | 10.89 | 3.81 | 12.45 | 2.30 | 7.23 | 6.07 | 4.53 | 1.25 | 5.93 | 1.00 | 0.72 | 0.69 |
| Brain | 0.08 | 0.02 | 0.04 | 0.01 | 0.03 | 0.01 | 0.01 | 0.01 | 0.00 | 0.00 | 0.00 | 0.00 | 0.00 | 0.00 |
| Tumor-kidney ratio | 0.35 | 0.03 | 1.28 | 0.52 | 2.68 | 0.36 | 8.96 | 6.30 | 11.13 | 3.02 | 10.08 | 2.69 | 4.78 | 4.74 |

TABLE 26
uptake values in different organs and tissues for ²²⁵Ac-labeled untagged VHH B1 up to 96 h post i.v. injection,
expressed as % injected activity per gram of tissue (% IA/g). Data presented as mean ± SD (n = 3).

| | 1 h | | 3 h | | 6 h | | 24 h | | 48 h | | 72 h | | 96 h | |
|---|---|---|---|---|---|---|---|---|---|---|---|---|---|---|
| Organ/tissue | MEAN | SD | MEAN | SD | MEAN | SD | MEAN | SD | MEAN | SD | MEAN | SD | MEAN | SD |
| Blood | 0.43 | 0.22 | 0.20 | 0.01 | 0.11 | 0.01 | 0.02 | 0.01 | 0.01 | 0.00 | 0.01 | 0.01 | 0.00 | 0.01 |
| Heart | 0.27 | 0.03 | 0.10 | 0.01 | 0.08 | 0.02 | 0.04 | 0.03 | 0.03 | 0.00 | 0.03 | 0.01 | 0.02 | 0.01 |
| Lung | 0.38 | 0.01 | 0.18 | 0.03 | 0.15 | 0.03 | 0.07 | 0.00 | 0.27 | 0.26 | 0.02 | 0.02 | 0.03 | 0.01 |
| Liver | 0.33 | 0.03 | 0.26 | 0.01 | 0.36 | 0.01 | 0.41 | 0.09 | 0.38 | 0.04 | 0.34 | 0.03 | 0.42 | 0.02 |
| Pancreas | 0.45 | 0.04 | 0.21 | 0.03 | 0.14 | 0.02 | 0.05 | 0.03 | 0.02 | 0.01 | 0.04 | 0.01 | 0.02 | 0.01 |
| Spleen | 0.20 | 0.03 | 0.11 | 0.01 | 0.11 | 0.03 | 0.12 | 0.05 | 0.08 | 0.02 | 0.06 | 0.01 | 0.06 | 0.01 |
| Adrenals | 0.48 | 0.11 | 0.25 | 0.09 | 0.17 | 0.13 | 0.20 | 0.07 | 0.10 | 0.07 | 0.14 | 0.10 | 0.30 | 0.18 |
| Kidney | 12.41 | 1.52 | 12.30 | 0.53 | 13.51 | 1.18 | 8.07 | 1.39 | 4.44 | 0.51 | 3.68 | 0.56 | 2.47 | 0.18 |
| Stomach | 0.26 | 0.04 | 0.13 | 0.02 | 0.15 | 0.06 | 0.06 | 0.00 | 0.04 | 0.01 | 0.03 | 0.02 | 0.04 | 0.01 |
| Small intestine | 0.22 | 0.03 | 0.16 | 0.05 | 0.15 | 0.07 | 0.03 | 0.03 | 0.03 | 0.02 | 0.03 | 0.01 | 0.02 | 0.01 |
| Large intestine | 0.34 | 0.04 | 0.20 | 0.03 | 0.24 | 0.09 | 0.06 | 0.01 | 0.09 | 0.03 | 0.07 | 0.07 | 0.06 | 0.03 |
| Uterus | 1.13 | 0.31 | 0.65 | 0.10 | 0.39 | 0.10 | 0.28 | 0.06 | 0.20 | 0.14 | 0.14 | 0.10 | 0.13 | 0.08 |
| Ovaries | 0.43 | 0.08 | 0.20 | 0.02 | 0.07 | 0.02 | 0.08 | 0.03 | 0.08 | 0.05 | 0.07 | 0.00 | 0.06 | 0.01 |
| Skin | 1.87 | 0.31 | 0.94 | 0.24 | 0.85 | 0.16 | 0.31 | 0.08 | 0.14 | 0.05 | 0.22 | 0.08 | 0.18 | 0.07 |
| Muscle | 0.45 | 0.10 | 0.25 | 0.03 | 0.21 | 0.04 | 0.09 | 0.04 | 0.04 | 0.03 | 0.13 | 0.15 | 0.03 | 0.02 |
| Bone | 1.34 | 0.16 | 0.72 | 0.65 | 0.72 | 0.12 | 0.73 | 0.44 | 0.50 | 0.18 | 0.50 | 0.22 | 0.25 | 0.09 |
| Joint | 2.31 | 0.68 | 1.06 | 0.20 | 0.96 | 0.12 | 0.78 | 0.22 | 0.57 | 0.27 | 0.79 | 0.26 | 0.48 | 0.05 |
| Tumor | 4.18 | 0.54 | 5.03 | 1.74 | 6.51 | 1.77 | 3.49 | 0.49 | 3.44 | 0.40 | 1.90 | 0.17 | 1.68 | 0.65 |
| Brain | 0.03 | 0.01 | 0.01 | 0.00 | 0.02 | 0.01 | 0.01 | 0.01 | 0.01 | 0.01 | 0.01 | 0.00 | 0.00 | 0.01 |
| Tumor-kdiney ratio | 0.34 | 0.07 | 0.41 | 0.14 | 0.48 | 0.09 | 0.43 | 0.02 | 0.79 | 0.19 | 0.53 | 0.11 | 0.62 | 0.29 |

TABLE 27
uptake values in different organs and tissues for ¹⁷⁷Lu-labeled untagged VHH B1 up to 96 h post i.v. injection,
expressed as % injected activity per gram of tissue (% IA/g). Data presented as mean ± SD (n = 3).

| | 1 h | | 4 h | | 6 h | | 24 h | | 48 h | | 72 h | | 96 h | |
|---|---|---|---|---|---|---|---|---|---|---|---|---|---|---|
| Organ/tissue | MEAN | SD | MEAN | SD | MEAN | SD | MEAN | SD | MEAN | SD | MEAN | SD | MEAN | SD |
| Blood | 0.89 | 0.07 | 0.43 | 0.07 | 0.27 | 0.06 | 0.06 | 0.02 | 0.04 | 0.01 | 0.01 | 0.01 | 0.01 | 0.01 |
| Heart | 0.33 | 0.01 | 0.20 | 0.03 | 0.13 | 0.01 | 0.06 | 0.02 | 0.03 | 0.01 | 0.02 | 0.01 | 0.01 | 0.00 |
| Lung | 0.48 | 0.09 | 0.27 | 0.07 | 0.20 | 0.03 | 0.12 | 0.07 | 0.08 | 0.04 | 0.03 | 0.01 | 0.03 | 0.00 |
| Liver | 0.39 | 0.08 | 0.36 | 0.03 | 0.37 | 0.11 | 0.23 | 0.06 | 0.79 | 1.13 | 0.09 | 0.01 | 0.06 | 0.02 |
| Pancreas | 0.81 | 0.08 | 0.33 | 0.06 | 0.25 | 0.05 | 0.07 | 0.01 | 0.04 | 0.01 | 0.02 | 0.00 | 0.01 | 0.00 |
| Spleen | 0.21 | 0.02 | 0.19 | 0.01 | 0.14 | 0.02 | 0.10 | 0.02 | 0.06 | 0.01 | 0.05 | 0.00 | 0.03 | 0.00 |
| Adrenals | 1.05 | 0.13 | 0.79 | 0.04 | 0.60 | 0.05 | 0.30 | 0.07 | 0.18 | 0.06 | 0.11 | 0.04 | 0.09 | 0.01 |
| Kidney | 28.94 | 4.27 | 42.27 | 1.58 | 42.16 | 4.41 | 28.38 | 2.02 | 17.73 | 1.51 | 8.87 | 1.36 | 5.02 | 0.53 |
| Stomach | 0.33 | 0.08 | 0.24 | 0.02 | 0.17 | 0.03 | 0.09 | 0.00 | 0.07 | 0.03 | 0.02 | 0.01 | 0.02 | 0.01 |
| Small intestine | 0.38 | 0.10 | 0.31 | 0.14 | 0.14 | 0.03 | 0.08 | 0.02 | 0.04 | 0.01 | 0.03 | 0.01 | 0.02 | 0.01 |
| Large intestine | 0.69 | 0.10 | 0.71 | 0.24 | 0.25 | 0.03 | 0.17 | 0.01 | 0.08 | 0.02 | 0.05 | 0.02 | 0.03 | 0.00 |
| Ovary | 1.17 | 0.42 | 0.63 | 0.31 | 0.42 | 0.07 | 0.36 | 0.07 | 0.19 | 0.03 | 0.12 | 0.03 | 0.13 | 0.01 |
| Uterus | 1.91 | 0.68 | 1.13 | 0.35 | 0.73 | 0.11 | 0.55 | 0.16 | 0.24 | 0.09 | 0.08 | 0.04 | 0.07 | 0.02 |
| Skin | 2.36 | 0.21 | 1.42 | 0.06 | 1.38 | 0.18 | 0.40 | 0.02 | 0.40 | 0.36 | 0.10 | 0.01 | 0.06 | 0.01 |
| Muscle | 0.79 | 0.06 | 0.50 | 0.12 | 0.60 | 0.10 | 0.15 | 0.00 | 0.06 | 0.01 | 0.05 | 0.01 | 0.02 | 0.01 |
| Bone | 1.34 | 0.20 | 1.15 | 0.23 | 1.07 | 0.46 | 0.61 | 0.12 | 0.21 | 0.02 | 0.14 | 0.03 | 0.12 | 0.01 |
| Joint | 2.83 | 0.44 | 1.88 | 0.13 | 1.56 | 0.24 | 0.77 | 0.02 | 0.40 | 0.00 | 0.18 | 0.07 | 0.17 | 0.01 |
| Tumor | 4.56 | 0.42 | 10.52 | 2.25 | 9.20 | 0.91 | 10.12 | 3.55 | 4.88 | 1.29 | 3.25 | 0.09 | 1.39 | 0.05 |
| Brain | 0.05 | 0.01 | 0.03 | 0.01 | 0.02 | 0.01 | 0.01 | 0.00 | 0.01 | 0.00 | 0.01 | 0.00 | 0.01 | 0.00 |
| Tumor-kidney ratio | 0.16 | 0.04 | 0.25 | 0.06 | 0.22 | 0.00 | 0.35 | 0.11 | 0.27 | 0.05 | 0.37 | 0.05 | 0.19 | 0.03 |

Taken together, this example indicates that targeting of natural human FAP-expression on human glioblastoma tumors (U87 MG) is feasible with ¹³¹I-labeled ²²⁵Ac-labeled and ¹⁷⁷Lu-labeled untagged VHH B1. The in vivo tumor targeting potential combined with limited retention of radioactivity in additional organs and tissues supports their therapeutic application. The most optimal biodistribution was obtained for the ¹³¹I-labeled variant of untagged VHH B1, with high and sustained tumor targeting with a fast washout from kidneys. ²²⁵Ac-labeled untagged VHH B1 revealed high and sustained tumor targeting combined with a slower clearance from kidneys. Finally, ¹⁷⁷Lu-labeled untagged VHH B1 efficiently targets hFAP-expressing human glioblastoma tumors but is retained in kidneys significantly more over time compared to the two other radiolabeled variants of untagged VHH B1.

### Example 11: Therapeutic Efficacy of Radiolabeled Variants of Untagged VHH B1

In this example we describe the therapeutic potential of ¹³¹I-labeled; ²²⁵Ac-labeled and ¹⁷⁷Lu-labeled untagged VHH B1, evaluated in mice with human FAP expressing tumors. In all three cases, mice with small established tumors were treated 6 consecutive times with either (i) a high or (ii) low dose of radiolabeled untagged VHH B1, (iii) high radioactive dose of radiolabeled R3B23 or finally (iv) vehicle solution. Tumor volume and animal weight were measured repeatedly. Dropouts were considered when one of the following endpoints was reached: for subcutaneous tumors (i) tumor size of >1500 mm³, (ii) >20% weight loss or (iii) the presence of necrotic tumor tissue.

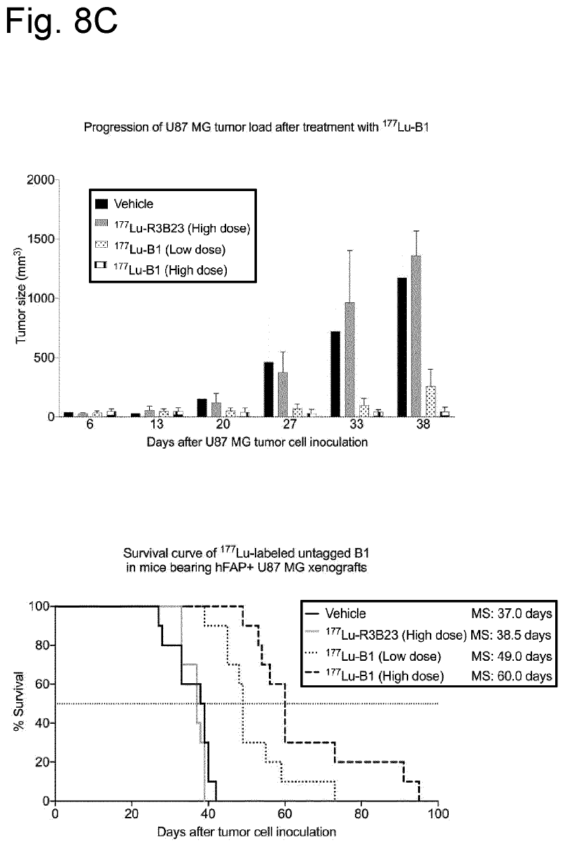

In the case of ¹³¹I-labeled untagged VHH B1, athymic nude mice (n=10 per group) were inoculated subcutaneously human glioblastoma tumors (U87 MG) that naturally express human FAP. When small tumors were established, mice were intravenously injected in the tail vein with about 6000 μCi (±5 μg) or 3000 μCi (±2.5 μg) ¹³¹I-labeled untagged VHH B1; 6000 μCi (±5 μg) ¹³¹I-labeled R3B23 or with vehicle solution. Mice treated with ¹³¹I-labeled untagged VHH B1 lived significantly longer compared to mice treated with ¹³¹I-labeled R3B23 or with vehicle solution (p<0.0001, Log-rank Mantel-cox test), as depicted in FIG. 8A.

Next, the therapeutic efficacy of ²²⁵Ac-labeled untagged VHH B1 was assessed in athymic nude mice (n=10 per group) bearing human FAP-expressing HEK-293 tumors. When small tumors were established, mice were intravenously injected in the tail vein with about 6.5 μCi (±5 μg) or 3.25 μCi (±2.5 μg) ²²⁵Ac-labeled untagged VHH B1; 6.5 μCi (±5 μg) ²²⁵Ac-labeled R3B23 or with vehicle solution. Mice treated with ²²⁵Ac-labeled untagged VHH B1 lived significantly longer compared to mice treated with ²²⁵Ac-labeled R3B23 or with vehicle solution (p<0.0001, Log-rank Mantel-cox test), as depicted in FIG. 8B. Moreover, A treatment with high radioactive ²²⁵Ac-labeled untagged VHH B1 was more effective compared to low radioactive ²²⁵Ac-labeled untagged VHH B1 (p<0.005, Log-rank Mantel-cox test).

Finally, the therapeutic efficacy of ¹⁷⁷Lu-labeled untagged VHH B1 was assessed in athymic nude mice (n=10 per group) bearing human glioblastoma tumors (U87 MG) that naturally express human FAP. When small tumors were established, mice were intravenously injected in the tail vein with about 3000 μCi (±5 μg) or 1500 μCi (±2.5 μg) ¹⁷⁷Lu-labeled untagged VHH B1; 3000 μCi (±5 μg) ¹⁷⁷Lu-labeled R3B23 or with vehicle solution. Mice treated with ¹⁷⁷Lu-labeled untagged VHH B1 lived significantly longer compared to mice treated with ¹⁷⁷Lu-labeled R3B23 or with vehicle solution (p<0.0001, Log-rank Mantel-cox test), as depicted in FIG. 8C. Moreover, A treatment with high radioactive ¹⁷⁷Lu-labeled untagged VHH B1 was more effective compared to low radioactive ¹⁷⁷Lu-labeled untagged VHH B1 (p<0.05, Log-rank Mantel-cox test).

In conclusion, ¹³¹I-labeled, ²²⁵Ac-labeled and ¹⁷⁷Lu-labeled untagged VHH B1 revealed to be effective in hFAP-expressing tumor xenografted mouse models. The optimal T/K ratio for ¹³¹I-labeled variant described in example 10 in combination with its good therapeutic potential makes this radiolabeled variant of untagged VHH B1 the preferred compound for theranostic use. Importantly, both ²²⁵Ac-labeled and ¹⁷⁷Lu-labeled untagged VHH B1 show to be effective in mice as well, however with less optimal T/K ratios as described in example 10.

1.-25. (canceled)

26. A compound comprising an antibody fragment that specifically binds human and/or murine FAP, wherein said antibody fragment comprises an amino acid sequence having at least 80% sequence identity with at least one of SEQ ID NO: 4, 8, 12, or 16, or at least 80% sequence identity over 50% of the length of said SEQ ID NO.

27. The compound of claim 26, wherein the antibody fragment comprises the CDRH1, CDRH2, and CDRH3 amino acid sequences of any one of the antibody fragment amino acid sequences set forth in SEQ ID NOs: 4, 8, 12, or 16.

28. The compound of claim 27, wherein the antibody fragment comprises the CDRH1, CDRH2, and CDRH3 amino acid sequences, respectively, set forth in SEQ ID NOs: 1, 2, and 3; 5, 6, and 7; 9, 10, and 11; or 13, 14, and 15.

29. The compound of claim 27, wherein the antibody fragment comprises an amino acid sequence having at least 80% sequence identity to the amino acid sequence of any one of SEQ ID NOs: 4, 8, 12, or 16.

30. The compound of claim 29, wherein the antibody fragment comprises the amino acid sequence of any one of SEQ ID NOs: 4, 8, 12, or 16.

31. The compound of claim 27, wherein the antibody fragment is conjugated to a radionuclide, cytotoxic payload, toxin, or detectable label, optionally wherein the radionuclide is chosen from the group consisting of a-emitting radionuclides, P-emitting radionuclides, positron-emitting radioisotopes (PET), and γ-emitting radioisotopes (SPECT).

32. The compound of claim 31, wherein the radionuclide is chosen from the group consisting of actinium-225, astatine-211, bismuth-212, bismuth-213, caesium-137, chromium-51, cobalt-60, copper-64, copper-67, dysprosium-165, erbium-169, fermium-255, fluorine-18, gallium-67, gallium-68, gold-198, holium-166, indium-111, iodine-123, iodine-124, iodine-125, iodine-131, iridium-192, iron-59, lead-212, lutetium-177, molybdenum-99, palladium-103, phosphorus-32, potassium-42, rhenium-186, rhenium-188, samarium-153, radium-223, radium-224, ruthenium-106, scandium-43, scandium-44, scandium-47, sodium-24, strontium-89, technetium-99m, terbium-149, terbium-161, terbium-149, thallium-201, thorium-227, xenon-133, ytterbium-169, ytterbium-177, yttrium-90, and zirconium-89.

33. The compound of claim 31, wherein the antibody fragment and radionuclide are conjugated by a linker.

34. The compound of claim 33, wherein the linker is a benzoate linker, optionally wherein the linker comprises N-succinimidyl-4-guanidinomethyl-3-[1-131]iodobenzoate (SGMIB) or a derivative thereof.

35. The compound of claim 27, wherein the antibody fragment:
(a) is linked to iodine-131 with a linker comprising SGMIB;
(b) is linked to lutetium-177 with a linker comprising 1,4,7,10-Tetraazacyclododecane-1,4,7,10-tetraacetic acid (DOTA) or 2-[Bis[2-[bis(carboxymethyl)amino]ethyl]amino]acetic acid (DTPA);
(c) is linked to actinium-225 with a linker comprising DOTA; or
(d) is linked to technetium-99m.

36. A composition comprising the compound of claim 27 and a pharmaceutically acceptable excipient.

37. A polynucleotide encoding the antibody fragment of claim 27, optionally wherein the polynucleotide is comprised within a vector.

38. A recombinant host cell comprising the polynucleotide of claim 37.

39. A method of producing a heavy chain antibody, the method comprising culturing the host cell of claim 38 under suitable conditions such that the polynucleotide is expressed, and the antibody is produced.

40. A method of treating cancer in a subject, the method comprising administering to the subject an effective amount of the compound of claim 27.

41. A method of detecting expression of FAP in a subject, the method comprising:
(a) administering the compound of claim 27 to a subject; and
(b) detecting binding of the compound to human and/or murine FAP in the subject.

42. A non-human animal comprising a nucleic acid construct allowing the expression of human FAP.

43. The compound of claim 26, wherein the antibody fragment is a heavy chain variable domain derived from a heavy chain antibody (VHH), or a fragment thereof.

44. The compound of claim 32, wherein the radionuclide is chosen from the group consisting of: Gallium-68 and Gallium-67.
